(19) United States
(12) Patent Application Publication
(10) Pub. No.: US 2025/0353825 A1
(43) Pub. Date: Nov. 20, 2025

(54) CANNABICYCLOL DERIVATIVES AND PREPARATION METHODS THEREOF

(71) Applicants: KUNMING UNIVERSITY OF SCIENCE AND TECHNOLOGY, Kunming (CN); WEST CHINA HOSPITAL, SICHUAN UNIVERSITY, Chengdu (CN)

(72) Inventors: Rongtao LI, Kunming (CN); Xudong ZHAO, Chengdu (CN); Hejiang LUO, Kunming (CN); Tao JIANG, Kunming (CN); Nan LIU, Chengdu (CN); Ye YU, Chengdu (CN)

(73) Assignees: KUNMING UNIVERSITY OF SCIENCE AND TECHNOLOGY, Kunming (CN); WEST CHINA HOSPITAL, SICHUAN UNIVERSITY, Chengdu (CN)

(21) Appl. No.: 19/281,809

(22) Filed: Jul. 28, 2025

Related U.S. Application Data
(63) Continuation of application No. PCT/CN2024/131495, filed on Nov. 12, 2024.

(30) Foreign Application Priority Data
Nov. 13, 2023 (CN) 202311501773.3
Apr. 30, 2024 (CN) 202410540159.6

(51) Int. Cl. C07D 311/94; A61K 31/352; A61K 31/4433; A61K 31/5355; C07D 333/10; C07D 405/12; C07D 407/12; C07D 413/12

(52) U.S. Cl. CPC C07D 311/94; A61K 31/352; A61K 31/4433; A61K 31/5355; C07D 333/10; C07D 405/12; C07D 407/12; C07D 413/12

(57) ABSTRACT

The present disclosure provides cannabicyclol derivatives and preparation methods thereof, belonging to the field of biomedical technology. In the present disclosure, citral (3,7-Dimethyl-2,6-octadienal) and derivatives thereof, and olive alcohol (1,3-Dihydroxy-5-pentylbenzene) and derivatives thereof are used as raw materials for the efficient synthesis of the cannabicyclol derivatives in a one-pot manner under an action of ethylenediamine and a photocatalyst.

### CROSS-REFERENCE TO RELATED APPLICATIONS

This application is a continuation of International Application No. PCT/CN2024/131495, filed on Nov. 12, 2024, which claims priority to Chinese Patent Application No. 202410540159.6, field on Apr. 30, 2024, entitled “Cannabicyclol derivates and methods for preparation thereof,” and Chinese Patent Application No. 202311501773.3, filed on Nov. 13, 2023, entitled “Applications of cannabicyclol in preparing medicines for killing cancer stem cells or inhibiting clonal formation of cancer stem cells,” the entire contents of each of which are incorporated herein by reference.

### TECHNICAL FIELD

The present disclosure relates to the field of biomedical technology, and in particular, to cannabicyclol derivatives and preparation methods thereof.

### BACKGROUND

Cancer poses a significant threat to human health. Currently, mainstream treatment approaches include surgery, radiotherapy, chemotherapy, immunotherapy, and targeted therapy. While surgery can be effective, it is an invasive procedure with notable postoperative recovery challenges, and most patients still require adjunctive pharmacological treatment. Radiotherapy and chemotherapy, on the other hand, are often associated with drug-related side effects and the development of resistance. In comparison, immunotherapy and targeted therapy generally cause fewer side effects; however, their high costs place a heavy financial burden on patients. Moreover, the efficacy of these therapies varies among individuals and is not universally applicable to all cancer types or patients. Therefore, developing novel anti-tumor drugs and treatment strategies is crucial for advancing cancer therapy.

Cancer stem cells (CSCs), characterized by their self-renewal and multipotent differentiation capabilities, can replicate indefinitely and give rise to various tumor cell types, making them key contributors to tumor initiation and progression. However, currently, there are very few specific targeted drugs available against CSCs. For example, Vismodegib targets a specific subpopulation of CSCs in basal cell carcinoma of the skin; LF3 inhibits the growth of colon cancer by suppressing the Wnt signaling pathway; and the INK inhibitor AS602801 has shown inhibitory effects on CSCs both in vitro and in vivo, yet still requires further development. Therefore, the development of novel therapeutics specifically targeting CSCs holds the potential to bring about a revolutionary breakthrough in cancer treatment.

Cannabicyclol, a non-psychoactive component of cannabinoids, has been relatively underexplored, primarily due to the difficulty of isolating and extracting it from natural sources, which has limited its application in studies of biological activity. Chinese Patent Application No. CN117379416A (Publication No. CN202311501773.3) was the first to propose the use of cannabicyclol in the preparation of medicines that kill or inhibit the clonal formation of CSCs, offering a new approach for CSC-targeted therapy. To address the challenge of isolating cannabicyclol from natural sources, Chinese Patent Application No. CN118530207A (Publication No. CN202410540159.6) disclosed a chemical synthesis method for cannabicyclol, overcoming limitations related to its low natural abundance and laying the groundwork for further exploration of its biological activity. Nevertheless, the development of more straightforward and efficient synthetic methods for cannabicyclol remains a focus of ongoing research.

### SUMMARY

The present disclosure provides a cannabicyclol derivative and a preparation method thereof to resolve the above problems.

To achieve the above invention purpose, the present disclosure provides the following scheme.

The present disclosure provides a cannabicyclol derivative, comprising a structural formula as follows:

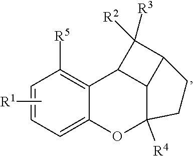

wherein R¹ is selected from hydrogen, substituted or unsubstituted linear alkyl, substituted or unsubstituted branched alkyl, substituted or unsubstituted aryl, and substituted or unsubstituted heteroaryl; R² and R³ are independently selected hydrogen, substituted or unsubstituted linear alkyl, substituted or unsubstituted branched alkyl, substituted or unsubstituted alkenyl, substituted or unsubstituted ester, substituted or unsubstituted heteroalkyl, substituted or unsubstituted aryl, and substituted or unsubstituted heteroaryl; R⁴ is selected from hydrogen, substituted or unsubstituted linear alkyl, substituted or unsubstituted branched alkyl, substituted or unsubstituted aryl, or substituted or unsubstituted heteroaryl, and R⁵ is selected from hydrogen, hydroxy, substituted or unsubstituted linear alkyl, substituted or unsubstituted branched alkyl, substituted or unsubstituted aryl, or substituted or unsubstituted heteroaryl.

In some embodiments, the cannabicyclol derivative is selected from compounds 3a to 3z, a compound 5, compounds 7a to 7i, compounds 9a to 9t, and compounds 13a to 13d. Structures of compounds 3a to 3z, the compound 5, the compounds 7a to 7i, the compounds 9a to 9t, and the compounds 13a to 13d are sequentially shown below:

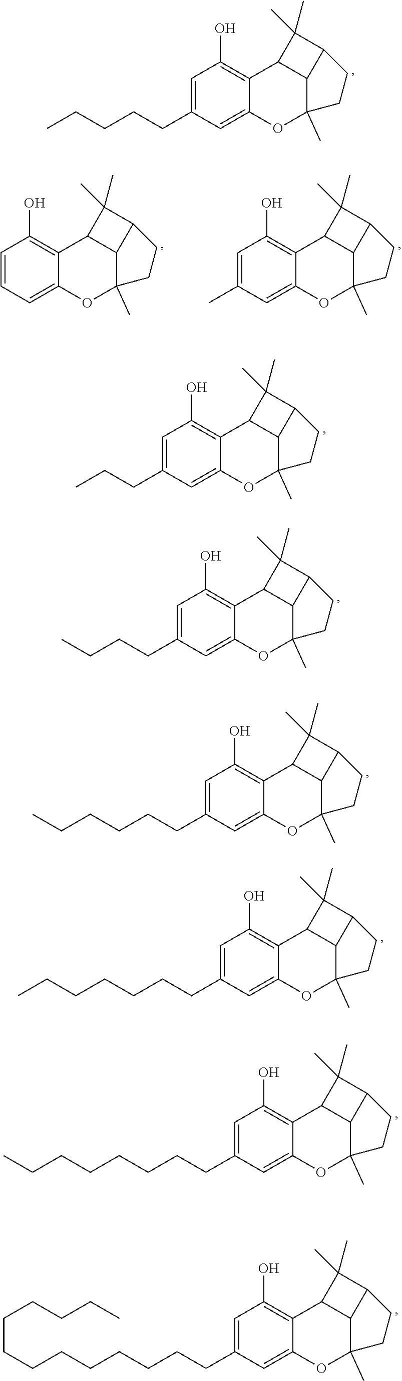

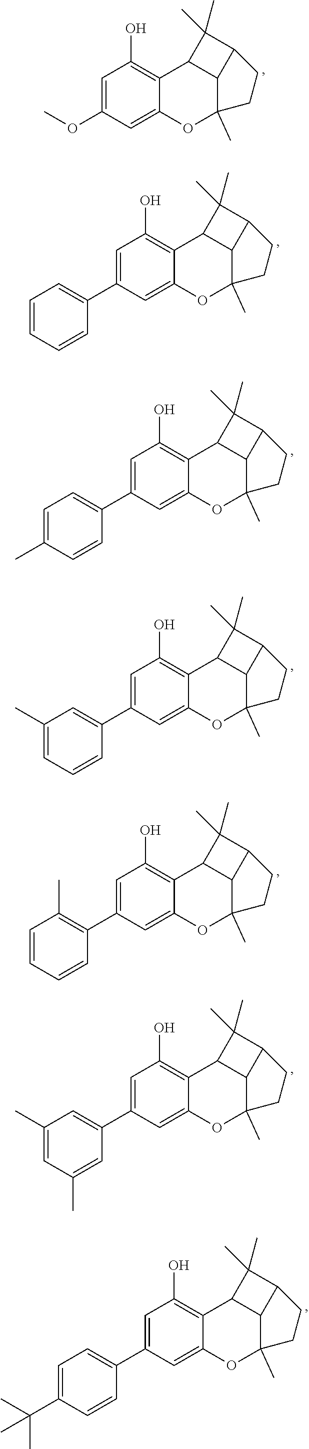

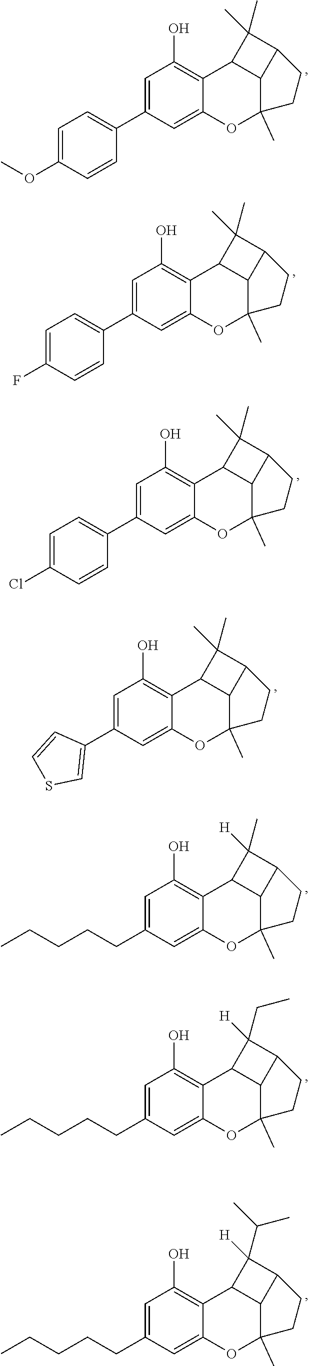

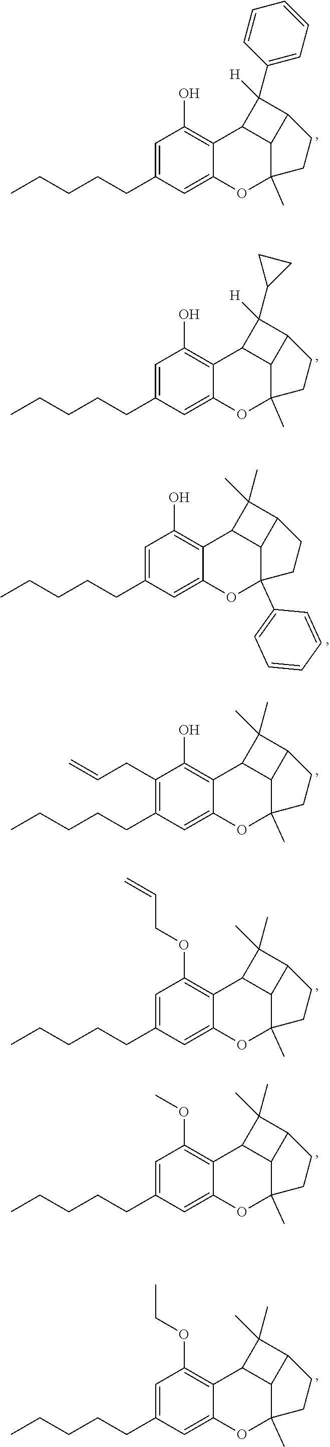

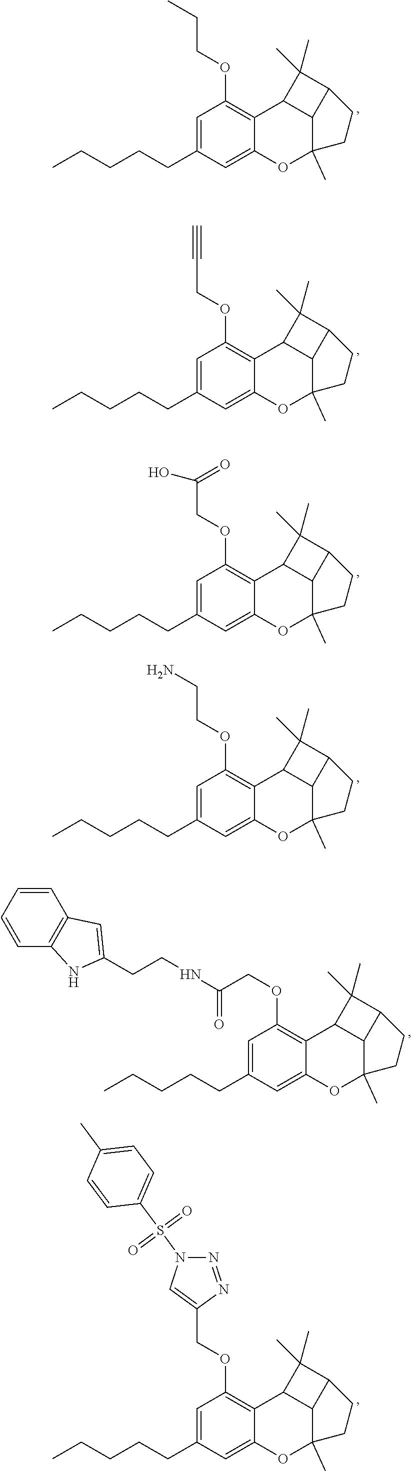

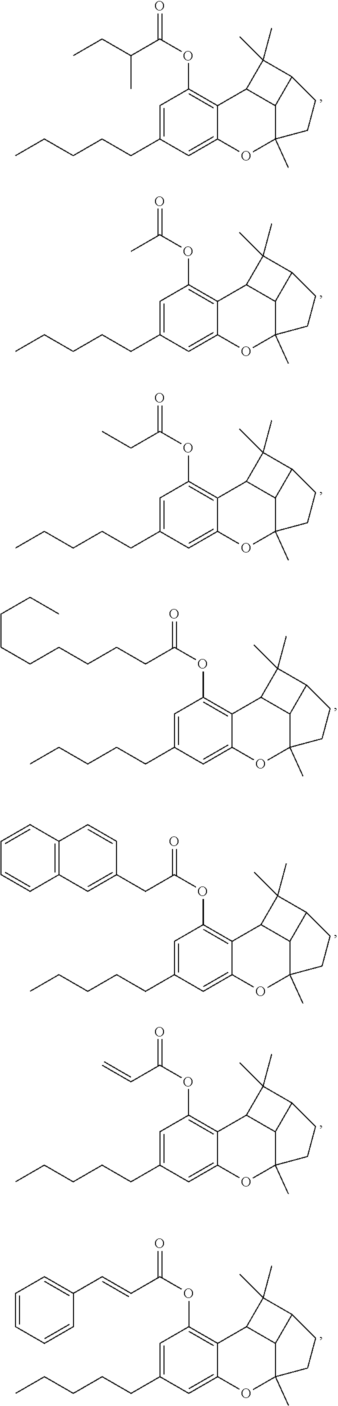

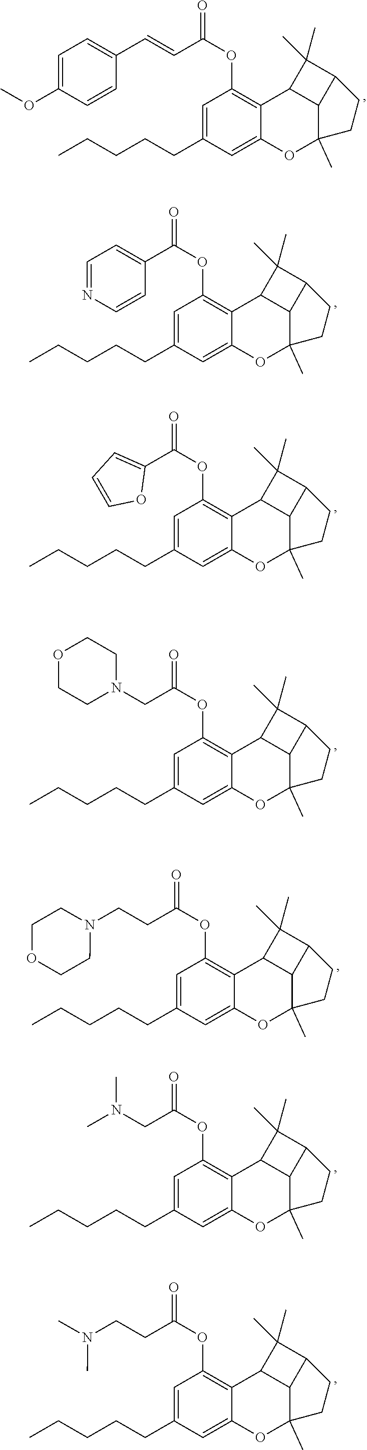

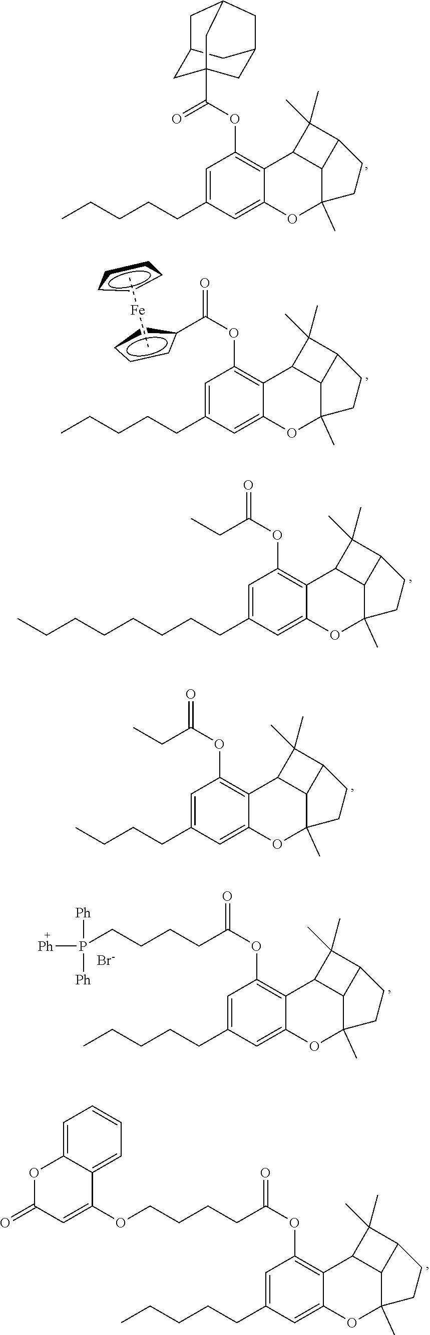

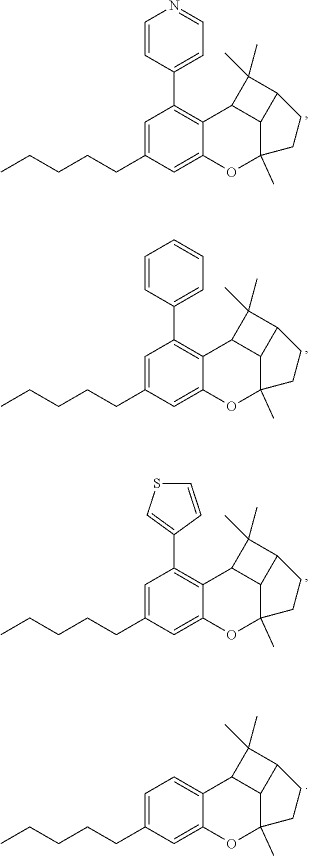 [SMILES: CCCCCc1cc2c(c(-c3ccncc3)c1)C1C3C(CCC3(C)O2)C1(C)C ; CCCCCc1cc2c(c(-c3ccccc3)c1)C1C3C(CCC3(C)O2)C1(C)C ; CCCCCc1cc2c(c(-c3ccsc3)c1)C1C3C(CCC3(C)O2)C1(C)C ; CCCCCc1ccc2c(c1)OC1(C)CCC3C1C2C3(C)C]

The present disclosure further provides a method for preparing a cannabicyclol derivative.

(1) Compounds 3a to 3z are prepared by the following steps.

Dissolving a compound of a formula I, a compound of a formula II, and ethylenediamine in toluene for reaction, and then conducting a photocatalytic reaction under a protective atmosphere and an action of a photocatalyst to obtain a cannabicyclol derivative of the formula III.

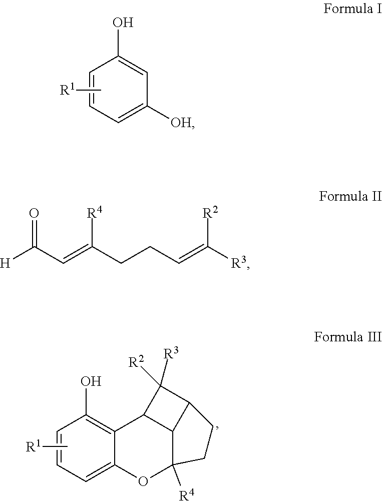

wherein R¹ is selected from hydrogen, substituted or unsubstituted linear alkyl, substituted or unsubstituted branched alkyl, substituted or unsubstituted aryl, and substituted or unsubstituted heteroaryl; R² and R³ are independently selected hydrogen, substituted or unsubstituted linear alkyl, substituted or unsubstituted branched alkyl, substituted or unsubstituted alkenyl, substituted or unsubstituted ester, substituted or unsubstituted heteroalkyl, substituted or unsubstituted aryl, and substituted or unsubstituted heteroaryl; R⁴ is selected from hydrogen, substituted or unsubstituted linear alkyl, substituted or unsubstituted branched alkyl, substituted or unsubstituted aryl, or substituted or unsubstituted heteroaryl, and R⁵ is selected from hydrogen, hydroxy, substituted or unsubstituted linear alkyl, substituted or unsubstituted branched alkyl, substituted or unsubstituted aryl, or substituted or unsubstituted heteroaryl.

In some embodiments, a cannabicyclol derivative includes a formula III:

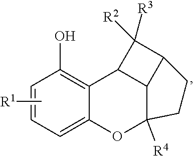

and the cannabicyclol derivative of the formula III is prepared by: dissolving a compound of a formula I

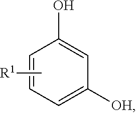

a compound of a formula II

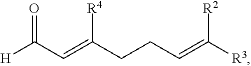

and ethylenediamine in toluene for reaction, and then conducting a photocatalytic reaction under a protective atmosphere and an action of a photocatalyst to obtain a cannabicyclol derivative of the formula III.
R¹ is selected from pentyl, R² is selected from methyl, R³ is selected from methyl, and R⁴ is selected from methyl;
R¹ is selected from methyl, R² is selected from methyl, R³ is selected from methyl, and R⁴ is selected from methyl;
R¹ is selected from butyl, R² is selected from methyl, R³ is selected from methyl, and R⁴ is selected from methyl;
R¹ is selected from hexyl, R² is selected from methyl, R³ is selected from methyl, and R⁴ is selected from methyl;
R¹ is selected from heptyl, R² is selected from methyl, R³ is selected from methyl, and R⁴ is selected from methyl;
R¹ is selected from octyl, R² is selected from methyl, R³ is selected from methyl, and R⁴ is selected from methyl;
R¹ is selected from n-tridecyl, R² is selected from methyl, R³ is selected from methyl, and R⁴ is selected from methyl;
R¹ is selected from phenyl, R² is selected from methyl, R³ is selected from methyl, and R⁴ is selected from methyl;
R¹ is selected from

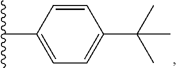 [SMILES: *c1ccc(C(C)(C)C)cc1]

R² is selected from methyl, R³ is selected from methyl, and R⁴ is selected from methyl;
R¹ is selected from H, R² is selected from methyl, R³ is selected from methyl, and R⁴ is selected from methyl;
R¹ is selected from propyl, R² is selected from methyl, R³ is selected from methyl, and R⁴ is selected from methyl;
R¹ is selected from methoxy, R² is selected from methyl, R³ is selected from methyl, and R⁴ is selected from methyl;
R¹ is selected from p-tolyl, R² is selected from methyl, R³ is selected from methyl, and R⁴ is selected from methyl;
R¹ is selected from m-tolyl, R² is selected from methyl, R³ is selected from methyl, and R⁴ is selected from methyl;
R¹ is selected from o-tolyl, R² is selected from methyl, R³ is selected from methyl, and R⁴ is selected from methyl;
R¹ is selected from

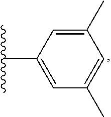 [SMILES: *c1cc(C)cc(C)c1]

R² is selected from methyl, R³ is selected from methyl, and R⁴ is selected from methyl;
R¹ is selected from

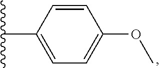 [SMILES: *c1ccc(OC)cc1]

R² is selected from methyl, R³ is selected from methyl, and R⁴ is selected from methyl;

R¹ is selected from

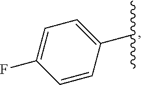 [SMILES: *c1ccc(F)cc1]

R² is selected from methyl, R³ is selected from methyl, and R⁴ is selected from methyl;
R¹ is selected from

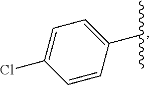 [SMILES: *c1ccc(Cl)cc1]

R² is selected from methyl, R³ is selected from methyl, and R⁴ is selected from methyl;
R¹ is selected from

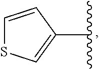 [SMILES: *c1ccsc1]

R² is selected from methyl, R³ is selected from methyl, and R⁴ is selected from methyl;
R¹ is selected from pentyl, R² is selected from H, R³ is selected from methyl, and R⁴ is selected from methyl;
R¹ is selected from pentyl, R² is selected from H, R³ is selected from ethyl, R⁴ is selected from methyl;
R¹ is selected from pentyl, R² is selected from H, R³ is selected from isopropyl, and R⁴ is selected from methyl;
R¹ is selected from pentyl, R² is selected from H, R³ is selected from phenyl, and R⁴ is selected from methyl;
R¹ is selected from pentyl, R² is selected from H, R³ is selected from cyclopropyl, and R⁴ is selected from methyl; or
R¹ is selected from pentyl, R² is selected from methyl, R³ is selected from methyl, and R⁴ is selected from phenyl.

(2) The compound 5 is prepared by the following steps.

Reacting a cannabinophene derivative and 2,3-dichloro-5,6-dicyanobenzoquinone under an action of indium trifluoromethanesulfonate to obtain a colorless oily liquid, the colorless oily liquid being the compound 5.

A structural formula of the cannabinophene derivative is:

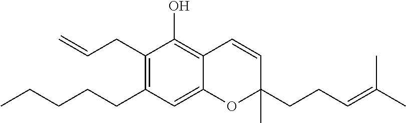 [SMILES: C=CCc1c(CCCCC)cc2c(c1O)C=CC(C)(CCC=C(C)C)O2]

(3) The compounds 7a to 7g are prepared by the following steps.

Mixing a cannabicyclol derivative of a structural formula III (e.g., the compound 3a

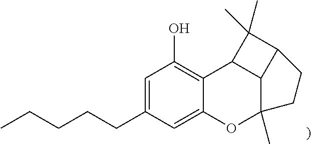 [SMILES: CCCCCc1cc(O)c2c(c1)OC1(C)CCC3C1C2C3(C)C]

with a halogen-containing compound and reacting under an action of a base, wherein the halogen-containing compound includes allyl bromide, halomethane, halogenated ethane, 1-halopropane, 3-bromopropyne, ethyl bromoacetate, and 2-(Boc-amino)ethyl bromide.

(4) The compound 7h is prepared by the following steps.

Mixing the compound 7f, tryptamine, 1-(3-dimethylaminopropyl)-3-ethylcarbodiimide hydrochloride, 1-hydroxybenzotriazole, and triethylamine and reacting to obtain a colorless oily liquid, the colorless oily liquid being the compound 7h;

(5) The compound 7i is prepared by the following steps.

Mixing the compound 7e, p-toluenesulfonyl azide, and thiophene-2-carboxylate Cuprous and reacting to obtain a colorless oily liquid, and colorless oily liquid being the compound 7i.

(6) The compounds 9a to 9p are prepared by the following steps.

Reacting a compound 3a and an acid under an action of a catalyst to obtain a colorless oily liquid, the colorless oily liquid being the compounds 9a to 9p.

A structural formula of the acid is

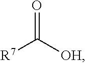

and R⁷ selects from 2-Methylbutyl, n-decyl, 2-Naphthylethyl, 3-Phenylpropenyl, (E)-4-Methoxy-3-phenylpropenyl, 4-Pyridylmethyl, 2-Furylmethyl, 4-Morpholinoethyl, 3-(4-Morpholinyl)propyl, N,N-Dimethylethyl, 3-(Dimethylamino)propyl, 1-Adamantylmethyl, and Ferrocene methyl, and the acid further includes acetic anhydride, propionyl chloride, acryloyl chloride, or propionic anhydride.

The compound 9q is prepared by reacting a compound 3h of a structural formula

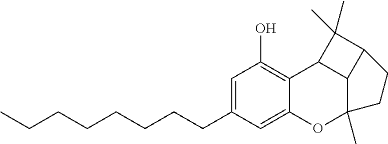 [SMILES: CCCCCCCCc1cc(O)c2c(c1)OC1(C)CCC3C1C2C3(C)C]

and propionic anhydride under an action of a catalyst to obtain the compound 9q.

The compound 9r is prepared by reacting a compound 3e of a structural formula

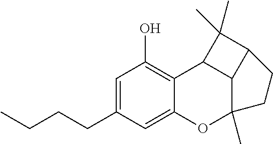 [SMILES: CCCCc1cc(O)c2c(c1)OC1(C)CCC3C1C2C3(C)C]

and propionic anhydride under an action of a catalyst to obtain the compound 9r.

(7) A compound 9s and a compound 9t are prepared by the following steps.

Reacting the compound 3a and 5-Bromopentanoic acid under an action of a catalyst to obtain an intermediate product, and reacting the intermediate product with a substituted compound to obtain the compound 9s and the compound 9t, the substituted compound including triphenylphosphine or 4-Hydroxycoumarin.

(8) The compounds 13a to 13c are prepared by the following steps.

Mixing a compound 3a of a structural formula

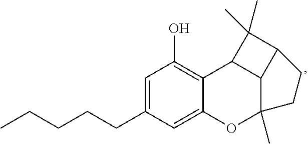 [SMILES: CCCCCc1cc(O)c2c(c1)OC1(C)CCC3C1C2C3(C)C]

triflic anhydride, and pyridine in a solvent for reaction to obtain an intermediate product, and reacting the intermediate product with a substituted boric acid under an action of a catalyst.

A structural formula of the intermediate product is:

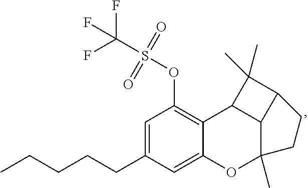 [SMILES: CCCCCc1cc2c(c(OS(=O)(=O)C(F)(F)F)c1)C1C3C(CCC3(C)O2)C1(C)C]

a structural formula of the substituted boric acid is:

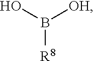

R⁸, and R⁸ is pyridine, phenyl, or thiophene.

(9) The compound 13d is prepared by the following steps.

Mixing the compound 3a, the triflic anhydride, and the pyridine in a solvent for reaction to obtain the intermediate product, and mixing the intermediate product, 1,1′-bis(diphenylphosphino) ferrocene, palladium acetate, formic acid, and triethylamine in a solvent for reaction to obtain the compound 13d.

The present disclosure further provides an application of the cannabicyclol derivative in the embodiments of the present disclosure in preparing a medicine for killing cancer stem cells or inhibiting the formation of cancer stem cells. The medicine comprises the cannabicyclol derivative of any one of the embodiments of the present disclosure and a pharmaceutically acceptable excipient, and the pharmaceutically acceptable excipient includes at least one of a diluent, an excipient, a filler, a binder, a humectant, a disintegrant, an absorption promoter, a surfactant, an adsorptive support, or a lubricant.

The present disclosure further provides an application of the cannabicyclol derivative in the embodiments of the present disclosure in preparing a medicine for killing cancer stem cells or inhibiting clonal formation of cancer stem cells.

In some embodiments, the cancer stem cells include glioblastoma stem cells, pancreatic cancer stem cells, and liver cancer stem cells.

In some embodiments, the glioblastoma stem cells include GSC-3, GSC-12, and GSC-18.

In some embodiments, the pancreatic cancer stem cells include PANC-1-CSC, BXPC-3-CSC, and ASPC1-CSC.

In some embodiments, the liver cancer stem cells include HepG2-CSC, MHCC97H-CSC, and SMMC7721-CSC.

The present disclosure provides an application of cannabicyclol in preparing an anti-tumor medicine, the cannabicyclol being applicable to killing cancer stem cells or inhibiting the clone formation of cancer stem cells.

In some embodiments, the tumors include glioblastoma, pancreatic cancer, and liver cancer.

The beneficial effects of the present disclosure include the following.

The present disclosure can synthesize cannabicyclol and a cannabicyclol derivative under mild reaction conditions. The process is simple, with straightforward steps and an atomic efficiency of up to 100%. Moreover, the precursor compounds of the cannabicyclol derivative can be cyclized from readily available and inexpensive citral and olive alcohol, making the process cost-effective. The method described herein achieves high yields of cannabicyclol and cannabicyclol derivatives under optimized conditions, significantly improving reaction efficiency and addressing issues related to the difficulty of natural product extraction and low separation yields.

The present disclosure reveals that cannabicyclol significantly and specifically kills a variety of cancer stem cells in vitro, including glioblastoma-derived GSC-3, GSC-12, and GSC-18, pancreatic cancer-derived PANC-1-CSC, BXPC-3-CSC, and ASPC1-CSC, and liver cancer-derived HepG2-CSC, MHCC97H-CSC, and SMMC7721-CSC. Further, the cannabicyclol can also inhibit the clone formation of a variety of cancer stem cells, such as GSC-3, GSC-12, HepG2-CSC, and SMMC7721-CSC. Therefore, the cannabicyclol is expected to be developed as an anti-tumor drug specifically targeting cancer stem cells. Additionally, in mouse in vivo experiments, the present disclosure demonstrates that the cannabicyclol can effectively inhibit liver cancer growth in mice, showing significant anti-liver cancer activity, indicating that the present disclosure can treat various cancers by targeting and killing various types of cancer stem cells.

### BRIEF DESCRIPTION OF THE DRAWINGS

To more clearly illustrate the technical solutions in the embodiments or prior art of the present disclosure, the accompanying drawings required to be used in the descriptions of the embodiments or prior art will be briefly described hereinafter, and it will be obvious that the accompanying drawings in the following descriptions are only embodiments of the present disclosure, and that a person of ordinary skill in the art can obtain other accompanying drawings according to the accompanying drawings provided, without exerting creative labor.

FIG. 1 shows a ¹H-NMR spectrum of a compound 3a in an application example 1;

FIG. 2 shows a ¹³C-NMR spectrum of the compound 3a in the application example 1;

FIG. 3 is a diagram showing cell viability of 9 types of cancer stem cells in solvents of compound 3a at different concentrations in an application example 2;

FIG. 4 is a diagram showing photographic results of cell clones after treatment of cancer stem cells with the compound 3a in an application example 4;

FIG. 5 is a diagram showing a change in tumor volume in mice at different dosing times in an application example 5;

FIG. 6 is a diagram showing tumor tissues removed from mice on day 23 in the application example 5; and

FIG. 7 is a diagram showing the average weights of tumors in different treatment groups in the application example 5, wherein the square-marked graph denotes a control group, the triangular-marked graph denotes a treatment group 1, and the circular-marked graph denotes a treatment group 2.

### DETAILED DESCRIPTION

The present disclosure provides a cannabicyclol derivative, comprising a structural formula as follows:

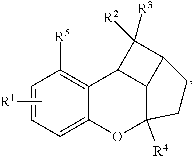

R¹ is selected from hydrogen, substituted or unsubstituted linear alkyl, substituted or unsubstituted branched alkyl, substituted or unsubstituted aryl, and substituted or unsubstituted heteroaryl; R² and R³ are independently selected hydrogen, substituted or unsubstituted linear alkyl, substituted or unsubstituted branched alkyl, substituted or unsubstituted alkenyl, substituted or unsubstituted ester, substituted or unsubstituted heteroalkyl, substituted or unsubstituted aryl, and substituted or unsubstituted heteroaryl; R⁴ is selected from hydrogen, substituted or unsubstituted linear alkyl, substituted or unsubstituted branched alkyl, substituted or unsubstituted aryl, or substituted or unsubstituted heteroaryl, and R⁵ is selected from hydrogen, hydroxy, substituted or unsubstituted linear alkyl, substituted or unsubstituted branched alkyl, substituted or unsubstituted aryl, or substituted or unsubstituted heteroaryl.

The linear alkyl refers to a group formed by the loss of one hydrogen atom from a linear alkane (a carbon chain without branches). A structural formula of the linear alkyl may be expressed as —CₙH₍₂ₙ₊₁₎.

The branched alkyl refers to an alkyl group that contains a branched structure, formed by the loss of one hydrogen atom from a branched alkane.

The alkenyl refers to an unsaturated hydrocarbon group containing one or more carbon-carbon double bonds (C═C). A structural formula of the alkenyl may be expressed as CₙH₍₂ₙ₋₁.

The ester refers to a functional group formed by the dehydration reaction between a carboxylic acid and an alcohol, and a structural formula of the ester may be expressed as —O—C(═O)—.

The heteroalkyl refers to a group containing heteroatoms (e.g., O, N, S, etc.) in the alkyl chain. For example, the heteroalkyl may include methoxy (—OCH₃), or the like.

The aryl refers to refers to any functional group or substituent derived (for example, by removal of one hydrogen atom) from an aromatic ring (typically aromatic hydrocarbons such as phenyl, naphthyl, biphenyl, indanyl, tetrahydronaphthyl, etc.). The aryl may be unsubstituted or substituted one or more times. Phenyl may be represented as -Ph.

The heteroaryl refers to an aryl group, as defined above, in which the aromatic ring contains one or more (e.g., 1-4) heteroatoms, typically oxygen, sulfur, or nitrogen. The heteroaryls are also known as “aromatic heterocycles” or “heteroaromatic compounds.” Examples of heteroaryl include those derived from pyrrole, furan, thiophene, thiazole, isothiazole, imidazole, triazole, tetrazole, pyrazole, oxazole, isoxazole, pyridine, pyrazine, diazine, pyrimidine, or the like.

In some embodiments, when there one or more substituents in R¹, R², R³, R⁴, and R⁵, the one or more substituents are independently selected from linear alkyl or branched alkyl containing 1 to 13 carbon atoms, halogen atom, linear alkoxy or branched alkoxy containing 1 to 13 carbon atoms, cyano, amino, amido, hydroxyl, ester, alkenyl, alkynyl, cycloalkyl containing 3 to 10 carbon atoms, heterocycloalkyl containing 3 to 10 carbon atoms, substituted or unsubstituted aryl, and substituted or unsubstituted heteroaryl.

The term “halogen” refers to halogen atoms, including fluorine, chlorine, bromine, and iodine.

In some embodiments, R¹, R², R³, R⁴, and R⁵ are independently selected from following structures:

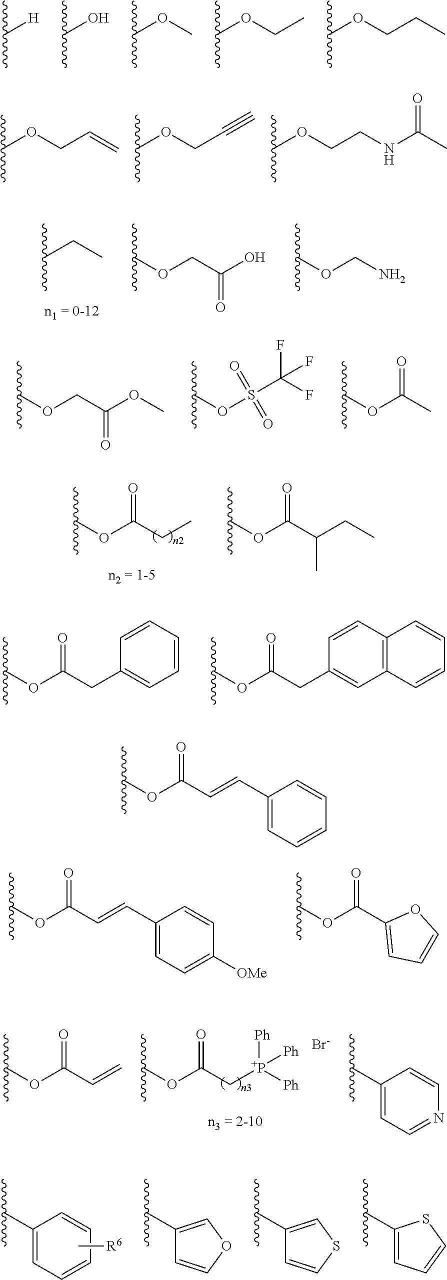

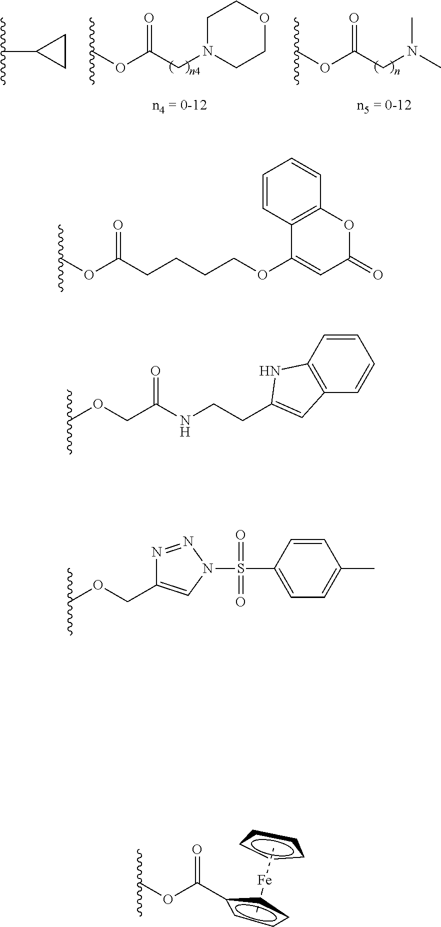

Where R⁶ is selected from following structures H, Me, Et, n-Pr, i-Pr, n-Bu, i-Bu, t-Bu, Ome, F, Cl, Br, and allyl.

In some embodiments, the cannabicyclol derivative is selected from compounds of following structural formulas:

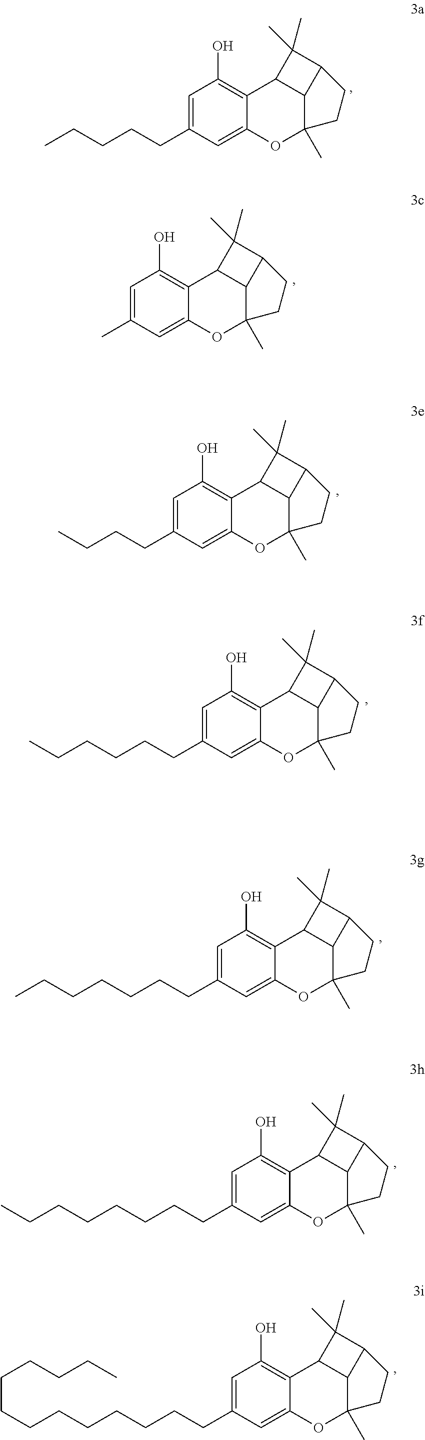

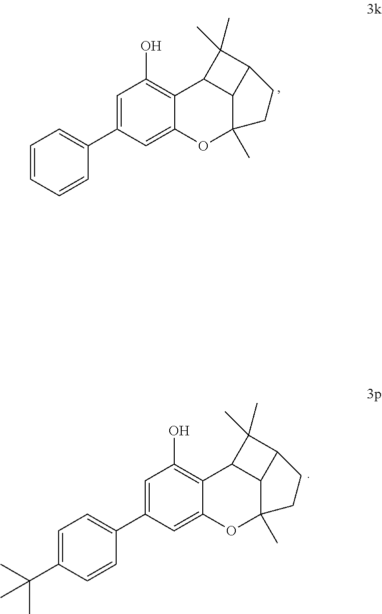 [SMILES: CC12CCC3C1C(c1c(O)cc(-c4ccccc4)cc1O2)C3(C)C ; CC(C)(C)c1ccc(-c2cc(O)c3c(c2)OC2(C)CCC4C2C3C4(C)C)cc1]

In some embodiments, the cannabicyclol derivative is selected form compounds of following structural formulas, the following structural formulas being 3b, 3d, 3j, 3l to 3i, 3q to 3z in sequence:

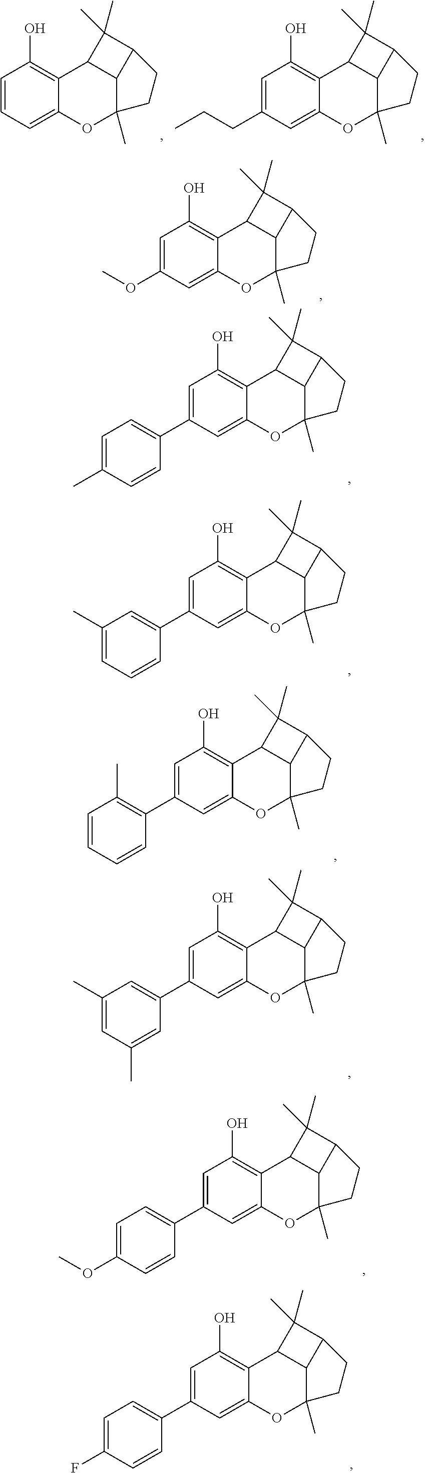

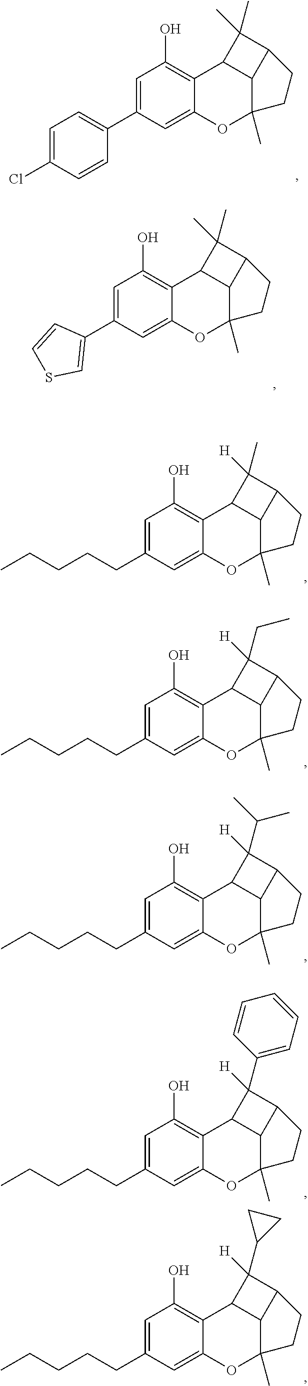

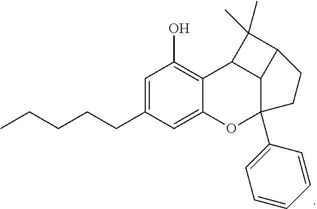 [SMILES: CCCCCc1cc(O)c2c(c1)OC1(c3ccccc3)CCC3C1C2C3(C)C]

In some embodiments, the cannabicyclol derivative comprises a compound of a following structural formula 5:

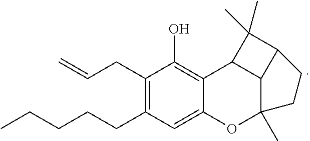 [SMILES: C=CCc1c(CCCCC)cc2c(c1O)C1C3C(CCC3(C)O2)C1(C)C]

In some embodiments, the cannabicyclol derivative is selected from compounds of following structural formulas, the following structural formulas being 7a to 7i in sequence:

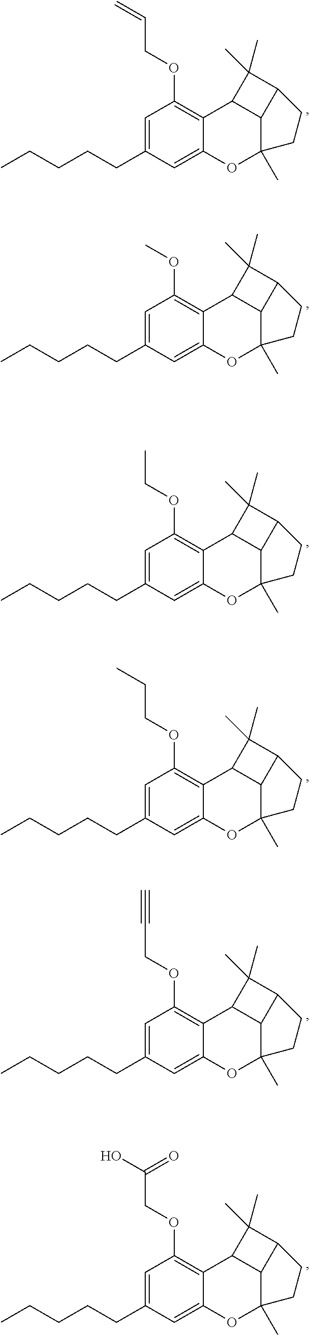 [SMILES: C=CCOc1cc(CCCCC)cc2c1C1C3C(CCC3(C)O2)C1(C)C ; CCCCCc1cc(OC)c2c(c1)OC1(C)CCC3C1C2C3(C)C ; CCCCCc1cc(OCC)c2c(c1)OC1(C)CCC3C1C2C3(C)C ; CCCCCc1cc(OCCC)c2c(c1)OC1(C)CCC3C1C2C3(C)C ; C#CCOc1cc(CCCCC)cc2c1C1C3C(CCC3(C)O2)C1(C)C ; CCCCCc1cc(OCC(=O)O)c2c(c1)OC1(C)CCC3C1C2C3(C)C]

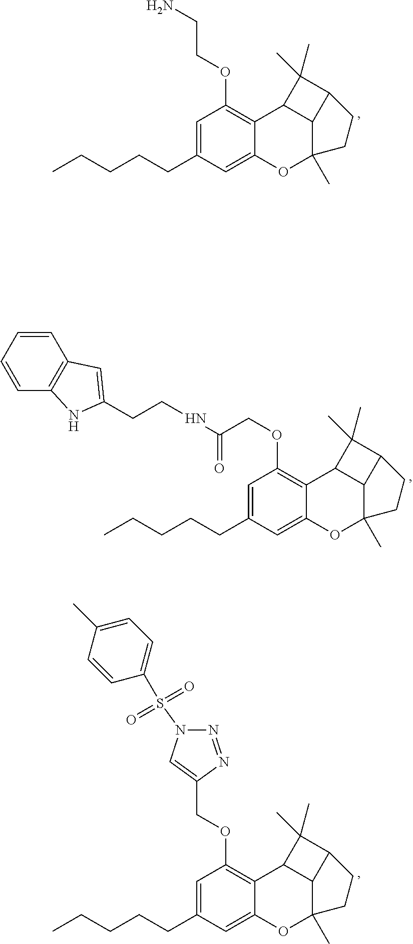 [SMILES: CCCCCc1cc(OCCN)c2c(c1)OC1(C)CCC3C1C2C3(C)C ; CCCCCc1cc(OCC(=O)NCCc2cc3ccccc3[nH]2)c2c(c1)OC1(C)CCC3C1C2C3(C)C ; CCCCCc1cc(OCc2cn(S(=O)(=O)c3ccc(C)cc3)nn2)c2c(c1)OC1(C)CCC3C1C2C3(C)C]

In some embodiments, the cannabicyclol derivative is selected from compounds of following structural formulas, the following structural formulas being 9a to 9t in sequence:

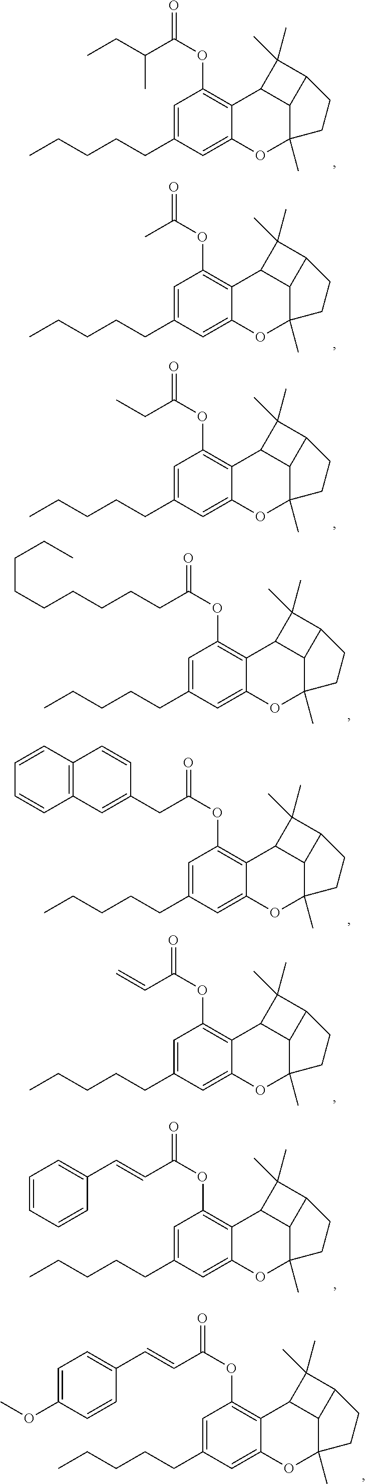

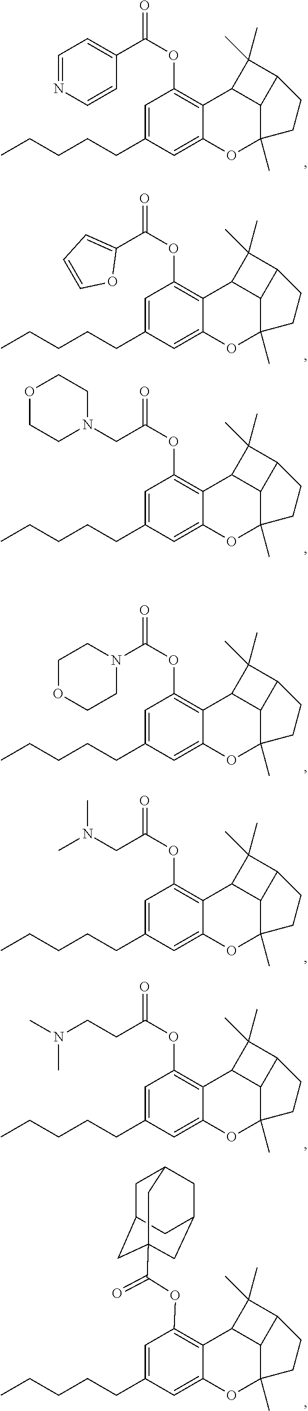

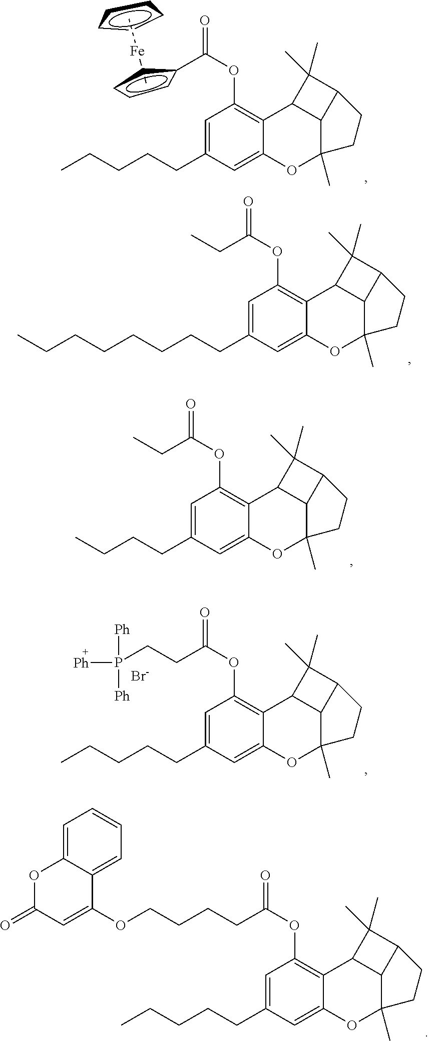

In some embodiments, the cannabicyclol derivative is selected from compounds of following structural formulas, the following structural formulas being 13a to 13d in sequence:

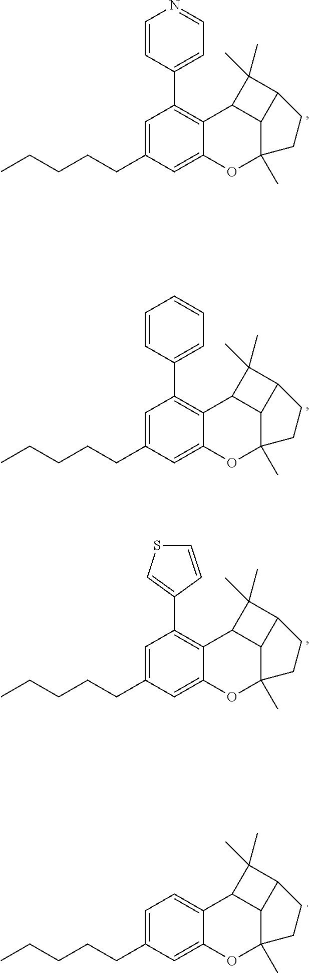 [SMILES: CCCCCc1cc2c(c(-c3ccncc3)c1)C1C3C(CCC3(C)O2)C1(C)C ; CCCCCc1cc2c(c(-c3ccccc3)c1)C1C3C(CCC3(C)O2)C1(C)C ; CCCCCc1cc2c(c(-c3ccsc3)c1)C1C3C(CCC3(C)O2)C1(C)C ; CCCCCc1ccc2c(c1)OC1(C)CCC3C1C2C3(C)C]

In the present disclosure, the numbers 3a to 3z, 5, 7a, to 7i, 9a to 9t, and 13a to 13d may denote a structural formula or a compound. A specific numbered structural formula may correspond to a specific numbered compound. For example, the structural formula 3a represent a structure of a compound 3a.

In some embodiments, the cannabicyclol derivative comprises a formula III:

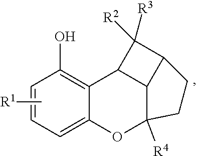

the cannabicyclol derivative of the formula III is prepared by: dissolving a compound of a formula I, a compound of a formula II, and ethylenediamine in toluene for reaction, and then conducting a photocatalytic reaction under a protective atmosphere and an action of a photocatalyst to obtain the cannabicyclol derivative of the formula III,

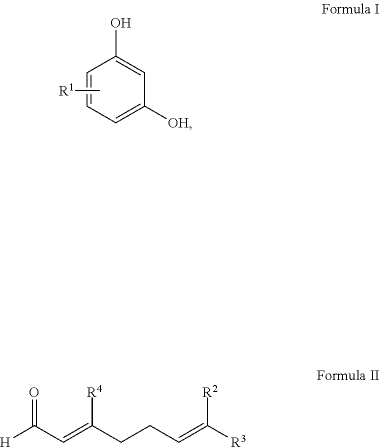

R¹ is selected from pentyl, R² is selected from methyl, R³ is selected from methyl, and R⁴ is selected from methyl;
R¹ is selected from methyl, R² is selected from methyl, R³ is selected from methyl, and R⁴ is selected from methyl;
R¹ is selected from butyl, R² is selected from methyl, R³ is selected from methyl, and R⁴ is selected from methyl;
R¹ is selected from hexyl, R² is selected from methyl, R³ is selected from methyl, and R⁴ is selected from methyl;
R¹ is selected from heptyl, R² is selected from methyl, R³ is selected from methyl, and R⁴ is selected from methyl;
R¹ is selected from octyl, R² is selected from methyl, R³ is selected from methyl, and R⁴ is selected from methyl;
R¹ is selected from n-tridecyl, R² is selected from methyl, R³ is selected from methyl, and R⁴ is selected from methyl;
R¹ is selected from phenyl, R² is selected from methyl, R³ is selected from methyl, and R⁴ is selected from methyl; or
R¹ is selected from

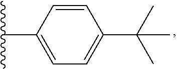 [SMILES: *c1ccc(C(C)(C)C)cc1]

R² is selected from methyl, R³ is selected from methyl, and R⁴ is selected from methyl.

In some embodiments, the cannabicyclol derivative comprises a formula III:

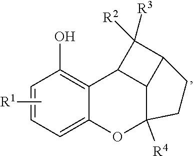

the cannabicyclol derivative of the formula III is prepared by: dissolving a compound of a formula I, a compound of a formula II, and ethylenediamine in toluene for reaction, and then conducting a photocatalytic reaction under a protective atmosphere and an action of a photocatalyst to obtain the cannabicyclol derivative of the formula III,

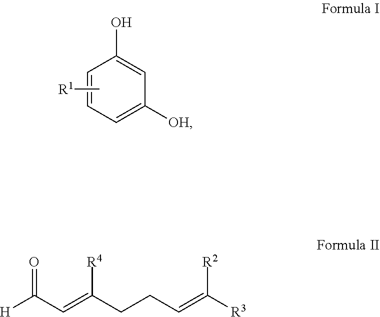

R¹ is selected from H, R² is selected from methyl, R³ is selected from methyl, and R⁴ is selected from methyl;
R¹ is selected from propyl, R² is selected from methyl, R³ is selected from methyl, and R⁴ is selected from methyl;
R¹ is selected from methoxy, R² is selected from methyl, R³ is selected from methyl, and R⁴ is selected from methyl;
R¹ is selected from p-tolyl, R² is selected from methyl, R³ is selected from methyl, and R⁴ is selected from methyl;
R¹ is selected from m-tolyl, R² is selected from methyl, R³ is selected from methyl, and R⁴ is selected from methyl;
R¹ is selected from o-tolyl, R² is selected from methyl, R³ is selected from methyl, and R⁴ is selected from methyl;
R¹ is selected from

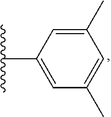 [SMILES: *c1cc(C)cc(C)c1]

R² is selected from methyl, R³ is selected from methyl, and R⁴ is selected from methyl;
R¹ is selected from

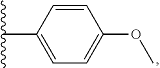 [SMILES: *c1ccc(OC)cc1]

R² is selected from methyl, R³ is selected from methyl, and R⁴ is selected from methyl;
R¹ is selected from

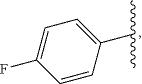 [SMILES: *c1ccc(F)cc1]

R² is selected from methyl, R³ is selected from methyl, and R⁴ is selected from methyl;
R¹ is selected from

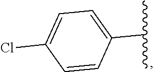 [SMILES: *c1ccc(Cl)cc1]

R² is selected from methyl, R³ is selected from methyl, and R⁴ is selected from methyl;
R¹ is selected from

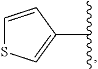 [SMILES: *c1ccsc1]

R² is selected from methyl, R³ is selected from methyl, and R⁴ is selected from methyl;
R¹ is selected from pentyl, R² is selected from H, R³ is selected from methyl, and R⁴ is selected from methyl;
R¹ is selected from pentyl, R² is selected from H, R³ is selected from ethyl, R⁴ is selected from methyl;
R¹ is selected from pentyl, R² is selected from H, R³ is selected from isopropyl, and R⁴ is selected from methyl;
R¹ is selected from pentyl, R² is selected from H, R³ is selected from phenyl, and R⁴ is selected from methyl;
R¹ is selected from pentyl, R² is selected from H, R³ is selected from cyclopropyl, and R⁴ is selected from methyl; or
R¹ is selected from pentyl, R² is selected from methyl, R³ is selected from methyl, and R⁴ is selected from phenyl.

In some embodiments, the protective atmosphere may include an inert gas such as nitrogen.

In some embodiments, the compound 5 is prepared by reacting cannabinophene derivative and 2,3-dichloro-5,6-dicyanobenzoquinone under an action of indium trifluoromethanesulfonate to obtain a colorless oily liquid, the colorless oily liquid being the compound 5. A structural formula of the cannabinophene derivative is:

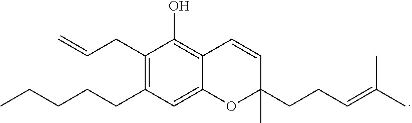 [SMILES: C=CCc1c(CCCCC)cc2c(c1O)C=CC(C)(CCC=C(C)C)O2]

In some embodiments, the compounds 7a to 7g are prepared by mixing a compound 3a of a structural formula

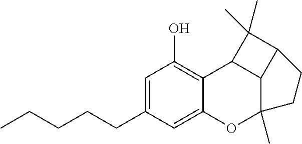 [SMILES: CCCCCc1cc(O)c2c(c1)OC1(C)CCC3C1C2C3(C)C]

with a halogen-containing compound and reacting under an action of a base, wherein the halogen-containing compound includes allyl bromide, halomethane, halogenated ethane, 1-halopropane, 3-bromopropyne, ethyl bromoacetate, and 2-(Boc-amino)ethyl bromide. The base includes potassium carbonate.

In some embodiments, the compound 7h is prepared by: mixing the compound 7f, tryptamine, 1-(3-dimethylaminopropyl)-3-ethylcarbodiimide hydrochloride, 1-hydroxybenzotriazole, and triethylamine and reacting to obtain a colorless oily liquid, the colorless oily liquid being the compound 7h.

In some embodiments, the compound 7i is prepared by: mixing the compound 7e, p-toluenesulfonyl azide, and thiophene-2-carboxylate Cuprous, and reacting to obtain a colorless oily liquid, and colorless oily liquid is the compound 7i.

In some embodiments, the compounds 9a to 9p are prepared by reacting a compound 3a of a structural formula

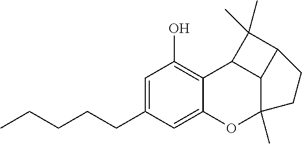 [SMILES: CCCCCc1cc(O)c2c(c1)OC1(C)CCC3C1C2C3(C)C]

and an acid under an action of a catalyst to obtain a colorless oily liquid, the colorless oily liquid being the compounds 9a to 9p. A structural formula of the acid is

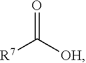

and R⁷ selects from 2-Methylbutyl, n-decyl, 2-Naphthylethyl, 3-Phenylpropenyl, (E)-4-Methoxy-3-phenylpropenyl, 4-Pyridylmethyl, 2-Furylmethyl, 4-Morpholinoethyl, 3-(4-Morpholinyl)propyl, N,N-Dimethylethyl, 3-(Dimethylamino)propyl, 1-Adamantylmethyl, or Ferrocene methyl. In some embodiments, the acid further includes acetic anhydride, propionyl chloride, acryloyl chloride, or propionic anhydride. The catalyst may include 4-Dimethylaminopyridine (DMAP) and N,N′-Dicyclohexylcarbodiimide (DCC). The reaction may be carried out in a solvent, and the solvent may include dichloromethane.

In some embodiments, the compound 9q is prepared by reacting a compound 3h of a structural formula

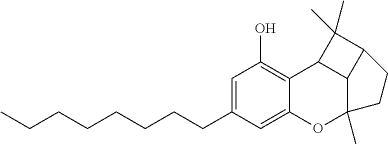 [SMILES: CCCCCCCCc1cc(O)c2c(c1)OC1(C)CCC3C1C2C3(C)C]

and propionic anhydride under an action of a catalyst to obtain the compound 9q. The catalyst may include 4-Dimethylaminopyridine (DMAP) and N,N′-Dicyclohexylcarbodiimide (DCC). The reaction may be carried out in a solvent, and the solvent may include dichloromethane.

In some embodiments, the compound 9r is prepared by reacting a compound 3e of a structural formula

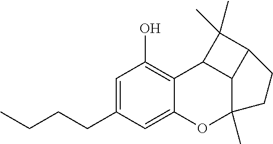 [SMILES: CCCCc1cc(O)c2c(c1)OC1(C)CCC3C1C2C3(C)C]

and propionic anhydride under an action of a catalyst to obtain the compound 9r. The catalyst may include 4-Dimethylaminopyridine (DMAP) and N,N′-Dicyclohexylcarbodiimide (DCC). The reaction may be carried out in a solvent, and the solvent may include dichloromethane.

In some embodiments, a compound 9s and a compound 9t are prepared by: reacting the compound 3a and 5-Bromopentanoic acid under an action of a catalyst to obtain an intermediate product, and reacting the intermediate product with a substituted compound to obtain the compound 9s and the compound 9t, the substituted compound including triphenylphosphine or 4-Hydroxycoumarin. The catalyst may include 4-Dimethylaminopyridine (DMAP) and N, N′-Dicyclohexylcarbodiimide (DCC). The reaction may be carried out in a solvent, and the solvent may include dichloromethane.

In some embodiments, compounds 13a to 13c are prepared by: mixing a compound 3a of a structural formula

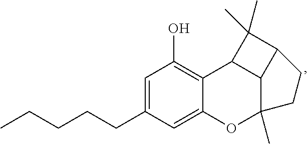 [SMILES: CCCCCc1cc(O)c2c(c1)OC1(C)CCC3C1C2C3(C)C]

triflic anhydride, and pyridine in a solvent for reaction to obtain an intermediate product, and reacting the intermediate product with a substituted boric acid under an action of a catalyst. The solvent may include dichloromethane, and the catalyst may include bis(triphenylphosphine)palladium(II) dichloride. A structural formula of the intermediate product is:

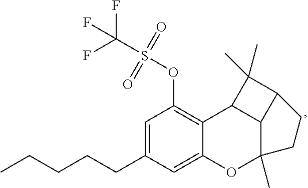 [SMILES: CCCCCc1cc2c(c(OS(=O)(=O)C(F)(F)F)c1)C1C3C(CCC3(C)O2)C1(C)C]

a structural formula of the substituted boric acid is:

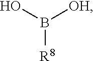

and R⁸ is pyridine, phenyl, or thiophene. The compound 13d is prepared by: mixing the compound

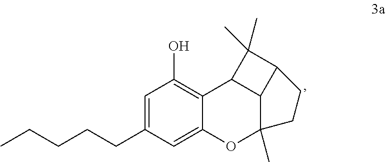 [SMILES: CCCCCc1cc(O)c2c(c1)OC1(C)CCC3C1C2C3(C)C]

the triflic anhydride, and the pyridine in a first solvent for reaction to obtain the intermediate product, and mixing the intermediate product, 1,1′-bis(diphenylphosphino) ferrocene, palladium acetate, formic acid, and triethylamine in a second solvent for reaction to obtain the compound 13d. The first solvent may include dichloromethane. The second solvent may include tetrahydrofuran.

In some embodiments, the compound of the formula I may include the following structures:

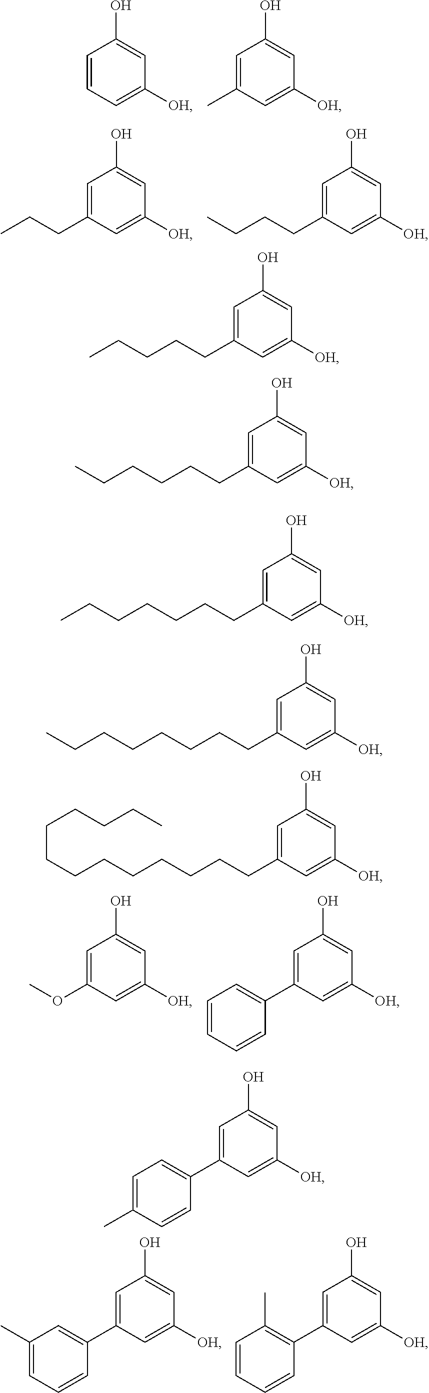

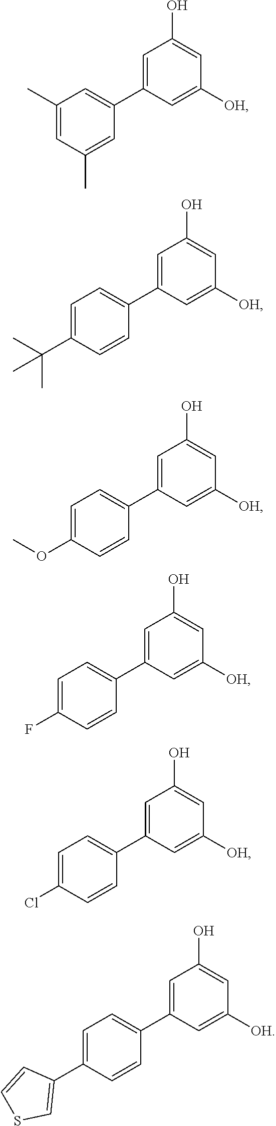 [SMILES: Cc1cc(C)cc(-c2cc(O)cc(O)c2)c1 ; CC(C)(C)c1ccc(-c2cc(O)cc(O)c2)cc1 ; COc1ccc(-c2cc(O)cc(O)c2)cc1 ; Oc1cc(O)cc(-c2ccc(F)cc2)c1 ; Oc1cc(O)cc(-c2ccc(Cl)cc2)c1 ; Oc1cc(O)cc(-c2ccc(-c3ccsc3)cc2)c1]

In some embodiments, the compound of the formula II may include citral,

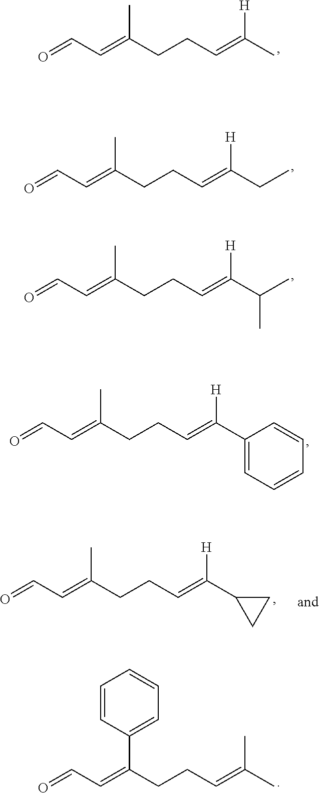

In some embodiments, the halogen-containing compound includes allyl bromide, halomethane, halogenated ethane, 1-halopropane, 3-bromopropyne, ethyl bromoacetate, and 2-(Boc-amino)ethyl bromide.

In some embodiments, in preparing the compounds 3a to 3z, the photocatalyst includes one or more of [Ir{dFCF₃ppy}₂(bpy)]PF₆, [Ir{dFCF₃ppy}₂(dtbbpy)]PF₆, [Ir(ppy)₂(dtbbpy)]PF₆, fac-Ir(ppy)₃, [Ru(bpy)₃]Cl₂, [Ru(bpy)₃](PF₆)₂ and Eosin Y. In some embodiments, the photocatalyst may be [Ir{dFCF₃ppy}₂(bpy)]PF₆. A light condition for a photocatalytic reaction includes a wavelength ranging from 365 nm to 560 nm, and an illumination time ranging from 30 min to 5 h.

A molar ratio of the compound of the formula I, the compound of the formula II, the ethylenediamine, and the photocatalyst is 1:1:(0.01 to 0.1):(0.005 to 0.02). In some embodiments, a molar ratio of the compound of the formula I, the compound of the formula II, the ethylenediamine, and the photocatalyst is 1:1:(0.02 to 0.05):(0.005 to 0.01).

In some embodiments, in preparing the compounds 7a to 7g, a molar ratio of the compound 3a, the halogen-containing compound, and the base is 1:2:(1-5). In some embodiments, the molar ratio of the compound 3a, the halogen-containing compound, and the base is 1:2:(2˜4). In some embodiments, the molar ratio of compound 3a, the halogen-containing compound, and the base is 1:2:3. In preparing the compounds 7a to 7g, a reaction temperature is in a range of 60° C. to 70° C. In some embodiments, in preparing the compounds 7a to 7g, the reaction temperature is 70° C. In preparing the compounds 7a to 7g, a reaction time is in a range of 5 h to 10 h. In some embodiments, in preparing the compounds 7a to 7g, the reaction time is 8 h.

In some embodiments, in preparing the compounds 9a to 9r, a molar ratio of the compound 3a, the acid, and the catalyst is 1:(1˜2):(1˜3). In preparing the compounds 9a to 9r, the molar ratio of the compound 3a, the acid, and the catalyst is 1:2:2. In preparing the compounds 9a to 9r, a reaction temperature is in a range of 20° C. to 40° C. In preparing the compounds 9a to 9r, the reaction temperature is 30° C. In preparing the compounds 9a to 9r, a reaction time is in a range of 5 h to 10 h. In preparing the compounds 9a to 9r, the reaction time is 5 h.

The present disclosure further provides a cannabicyclol derivative, comprising a structural formula as follows:

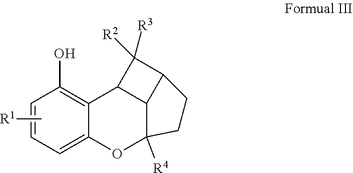

wherein R¹ is hydrogen, linear alkyl, branched alkyl, and heteroalkyl, R² is hydrogen, linear alkyl, branched alkyl, and heteroalkyl, R³ and R⁴ are independently selected hydrogen, linear alkyl, branched alkyl, alkenyl, ester, and heteroalkyl.

The present specification further provides a method for preparing a cannabicyclol derivative, comprising the following steps.

Step 1) diphenol compound and aldehyde compound are subjected to a cyclization reaction under an action of a catalyst to obtain product 1.

Step 2) the product 1,2,3-dichloro-5,6-dicyano-1,4-benzoquinone are reacted under an action of the catalyst to obtain the cannabicyclol derivative.

In the present disclosure, a reaction process of the cannabicyclol derivative may be as follows:

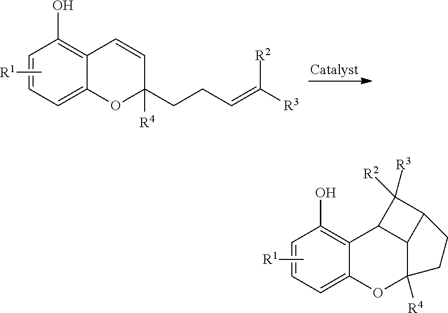

Wherein R¹ is hydrogen, linear alkyl, branched alkyl, and heteroalkyl, R² is hydrogen, linear alkyl, branched alkyl, and heteroalkyl, R³ and R⁴ are independently selected hydrogen, linear alkyl, branched alkyl, alkenyl, ester, and heteroalkyl.

In the present disclosure, a preparation process of the cannabicyclol derivative may be as follows:

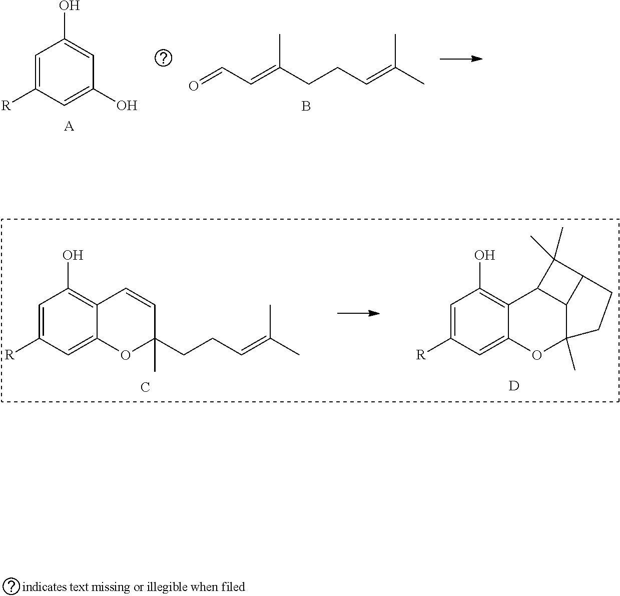

In some embodiments, the diphenol compound includes one of olive alcohol, resorcinol, 3-Methylresorcinol, 3-Butylresorcinol, 3-Hexylresorcinol, 3-Heptylresorcinol, 3-Octylresorcinol, 3-Tridecylresorcinol, 3-Phenylresorcinol, and 3-(4-Tert-butylphenyl)-resorcinol.

In some embodiments, a molar ratio of the diphenol compound, the aldehyde compound, and the catalyst in step 1) is (1 to 1.5):(1 to 1.5):(0.1 to 0.3). In some embodiments, the molar ratio of the diphenol compound, the aldehyde compound, and the catalyst in step 1) is 1:1.5:0.1 or 1.5:1:0.1.

In some embodiments, the catalyst in step 1) includes ethylenediamine. The aldehyde compound includes citral.

In some embodiments, in step 1), the cyclization reaction is carried out in a solvent. The solvent may be toluene. A dosage ratio of the solvent and the diphenol compound may be (2 mL to 5 mL):(0.2 mmol to 3 mmol). The dosage ratio of the solvent and the diphenol compound may be (3 mL to 4 mL):(0.35 mmol to 2 mmol). The dosage ratio of the solvent and the diphenol compound may be 3 mL:(1 mmol to 1.8 mmol).

In some embodiments, a cyclization reaction temperature in step 1) is in a range of 100° C. to 120° C. In some embodiments, the cyclization reaction temperature in step 1) is 110° C. In some embodiments, a cyclization reaction time in step 1) is in a range of 1 h to 10h. In some embodiments, the cyclization reaction time in step 1) is in a range of 2h to 8h. In some embodiments, the cyclization reaction time in step 1) is in a range of 3h to 6h.

In some embodiments, a molar ratio of the product 1, the product 1,2,3-dichloro-5,6-dicyano-1,4-benzoquinone, and the catalyst in step 2) is 1.0:(0.01 to 0.2):(0.1 to 0.2). In some embodiments, the molar ratio of the product 1, the product 1,2,3-dichloro-5,6-dicyano-1,4-benzoquinone, and the catalyst in step 2) is 1.0:0.1:0.1.

In some embodiments, the catalyst in step 2) includes a metal salt catalyst. The metal salt catalyst includes one or more of alkanoates, halides, acetylacetones, triflates, nitrates, sulfates, tetrafluoroborates, and perfluoroalkanoates. In some embodiments, the catalyst in step 2) is a metal salt catalyst of Fe, Yb, Sn, Bi, In, Sc, Ag, Zn, Cu, or Pd. In some embodiments, in step 2), the catalyst may be one or more of Fe₂(SO₄)₃, FeBr₃, FeCl₃, FeCl₂, Fe(acac)₃, Fe(OTf)₃, Fe(OTf₂, Yb(NO₃)₃, YbCl₃, Yb(CH₃COO)₃, Yb(OTf)₃, SnSO₄, Sn(OTf)₃, Bi(NO₃)₃, Bi(OTf)₃, In₂(SO₄)₃, InBr₃, In(CH₃COO)₃, In(OTf)₃, InCl₃, Sc(CH₃COO)₃, Sc(OTf)₃, AgBF₄, AgOTf, Zn(OTf)₂, Zn(BF₄)₂, ZnI, CuBr, CuI, Cu(OTf)₂, and CuCl.

In some embodiments, in step 2), the reaction is carried out in a solvent. The solvent includes one or more of dichloroethane, tetrahydrofuran, 1,4-Dioxane, dichloromethane, toluene, chlorobenzene, fluorobenzene, acetonitrile, methanol, water, triethylamine, dimethylformamide, n-hexane, ethyl acetate, and hydrochloric acid. In some embodiments, the solvent in step 2) includes one or more of dichloromethane, toluene, chlorobenzene, fluorobenzene, acetonitrile, methanol, water, triethylamine, dimethylformamide, n-hexane, ethyl acetate, and hydrochloric acid.

In some embodiments, a reaction temperature in step 2) is in a range of 0° C. to 100° C. In some embodiments, the reaction temperature in step 2) is in a range of 20° C. to 80° C. In some embodiments, the reaction temperature in step 2) is in a range of 40° C. to 60° C. In some embodiments, in step 2), a reaction time is in a range of 1 h to 2 h. In some embodiments, in step 2), the reaction time is 1.5 h.

The present disclosure also provides a medicine, comprising the cannabicyclol derivative in the above embodiments and a pharmaceutically acceptable excipient. The pharmaceutically acceptable excipient includes at least one of a diluent, an excipient, a filler, a binder, a humectant, a disintegrant, an absorption promoter, a surfactant, an adsorptive support, or a lubricant.

The present disclosure also provides an application of the cannabicyclol derivative in the above embodiments in preparing a medicine that kills cancer stem cells or inhibits the formation of cancer stem cells. The medicine further includes a pharmaceutically acceptable excipient, and the pharmaceutically acceptable excipient includes at least one of a diluent, an excipient, a filler, a binder, a humectant, a disintegrant, an absorption promoter, a surfactant, an adsorptive support, or a lubricant.

In the present disclosure, the cancer stem cells include glioblastoma stem cells, pancreatic cancer stem cells, and liver cancer stem cells.

The glioblastoma stem cells include GSC-3, GSC-12, and GSC-18.

The pancreatic cancer stem cells include PANC1-CSC, BXPC-3-CSC, and ASPC1-CSC.

The liver cancer stem cells include HepG2-CSC, MHCC97H-CSC, and SMMC7721-CSC.

The technical solutions provided herein are described in detail below in connection with embodiments, but they are not to be construed as limiting the scope of protection of the present disclosure.

The present disclosure employs four manners to synthesize olive alcohol analogs and citral analogs:

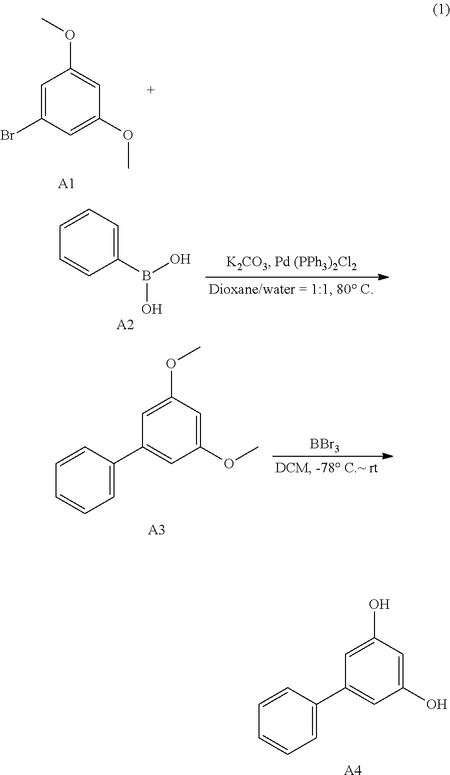

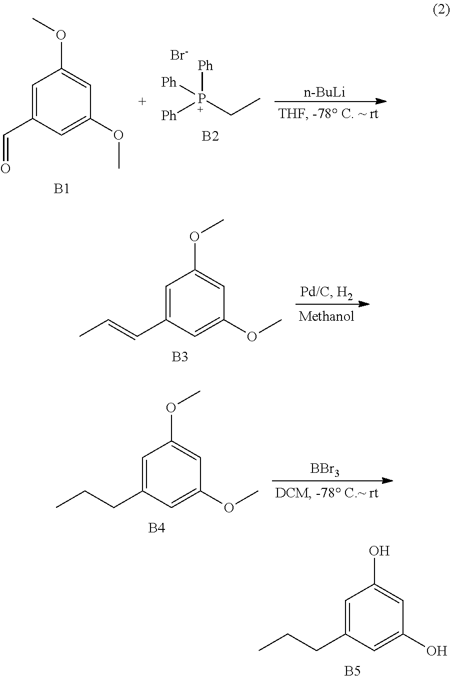

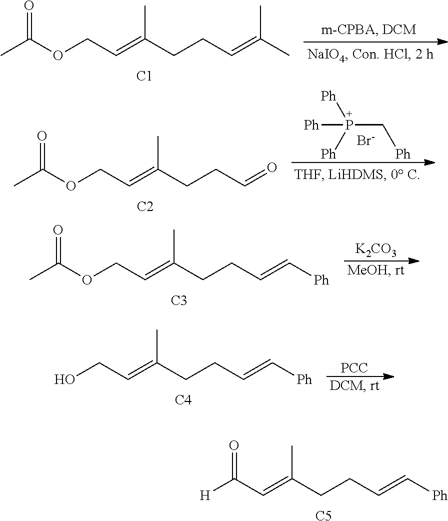

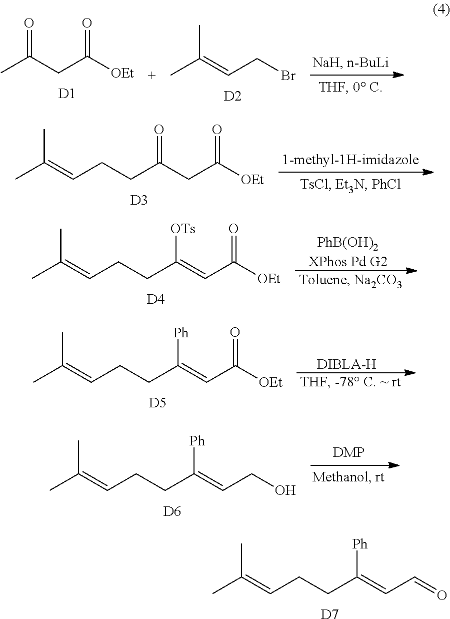

All four of the four manners are reported in the literature or patents, specified as follows.

Synthesis manner (1) is disclosed in Chinese Patent Application No. CN112334466A.

Synthesis manner (2) is disclosed in Synthesis, antiepileptic effects, and structure-activity relationships of α-asarone derivatives: In vitro and in vivo neuroprotective effect of selected derivatives. Bioorgan. Chem., 2021, 115, 105179.

Synthesis manner (3) is disclosed in A Tandem Cross-Metathesis/Semipinacol Rearrangement Reaction. Org. Lett., 2012, 14, 2462.

Synthesis manner (4) is disclosed in Enantioselective Tail-to-Head Cyclizations Catalyzed by Dual-Hydrogen-Bond Donors. J. Am. Chem. Soc, 2020, 142, 6951.

The entire contents of each of the four synthesis manners (1) to (4) are incorporated by reference into the present disclosure.

A synthesis method of a cannabicyclol derivative of a formula III disclosed in the present disclosure is as follows:

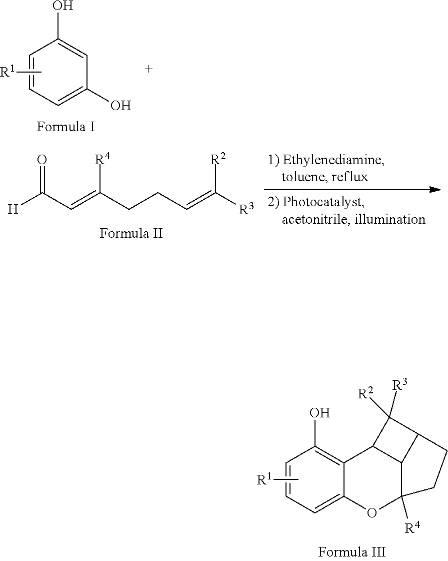

### Example 1

### Synthesis of C-7 Modified Cannabicyclol Derivatives

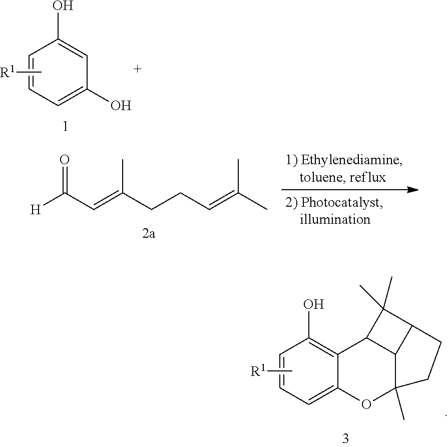

Taking a compound 3a of a formula

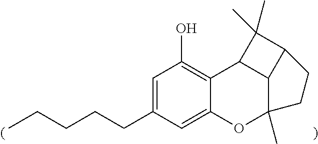 [SMILES: CCCCCc1cc(O)c2c(c1)OC1(C)CCC3C1C2C3(C)C]

as an example, a synthesis procedure may include: olive alcohol 1 (180 mg, 1 mmol, 1.0 equiv.), citral 2 (152 mg, 1 mmol, 1.0 equiv.), and ethylenediamine (7 μL, 10 mol %, 0.01 equiv.) were added to 5 mL of toluene under a nitrogen atmosphere to obtain a reaction mixture, then the reaction mixture was heated to reflux and reacted for 3 hours. Under the nitrogen atmosphere, after confirming completion of a reaction by thin-layer chromatography (TLC), the reaction mixture was stirred at room temperature. A photocatalyst [Ir{dFCF₃ppy}₂(bpy)]PF₆ (2 mg, 2 mol ‰, 0.005 equiv.) was added to the reaction mixture. Then the reaction mixture was irradiated with a 405 nm LED light source for 3 hours (alternatively, the reaction mixture was irradiated by sunlight for 5 days).

After confirming the completion of the reaction by TLC, the reaction mixture was extracted with 25 mL of ethyl acetate three times, and the organic phase was collected. After concentrating the organic phase through rotary evaporation, the organic phase was purified by silica gel column chromatography, yielding 62.2 mg of a colorless oily liquid (yield 39%, yield of 30% under sunlight irradiation), i.e., the compound 3a. Nuclear Magnetic Resonance (NMR) data of the compound 3a includes: ¹H NMR (600 MHz, CDCl₃) δ 6.33 (s, 1H), 6.18 (s, 1H), 4.48 (s, 1H), 3.06 (d, J=9.6 Hz, 1H), 2.57 (m, 1H), 2.50-2.42 (m, 2H), 2.39 (t, J=7.4 Hz, 1H), 2.02-1.93 (m, 1H), 1.71-1.65 (m, 1H), 1.65-1.52 (m, 4H), 1.38 (s, 3H), 1.37 (s, 3H), 1.34-1.25 (m, 4H), 0.88 (t, J=6.8 Hz, 3H), 0.80 (s, 3H).

Using the synthesis method, a series of C-7 modified cannabicyclol derivatives may be synthesized, including the following cannabicyclol derivatives.

Compound 3b:

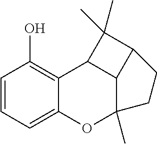 [SMILES: CC12CCC3C1C(c1c(O)cccc1O2)C3(C)C]

(raw material including:

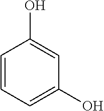

and citral (citral was used for preparing compounds 3a to 3t)), NMR data of the compound 3b includes: ¹H NMR (500 MHz, CD₃OD) δ 6.86 (t, J=8.0 Hz, 1H), 6.32 (dd, J=8.0, 1.1 Hz, 1H), 6.29 (dd, J=8.1, 1.1 Hz, 1H), 3.13 (d, J=9.6 Hz, 1H), 2.53 (d, J=7.3 Hz, 1H), 2.38 (t, J=7.5 Hz, 1H), 2.02-1.92 (m, 1H), 1.70-1.53 (m, 3H), 1.36 (s, 3H), 1.33 (s, 3H), 0.76 (s, 3H). A yield of the compound 3b was 37%.

Compound 3c:

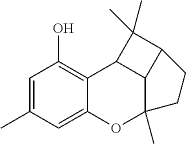 [SMILES: Cc1cc(O)c2c(c1)OC1(C)CCC3C1C2C3(C)C]

(raw material including:

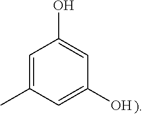

NMR data of the compound 3c includes: ¹H NMR (600 MHz, CD₃OD) δ 6.11 (s, 1H), 6.07 (s, 1H), 3.02 (d, J=9.6 Hz, 1H), 2.46 (t, J=8.0 Hz, 1H), 2.30 (t, J=7.5 Hz, 1H), 2.11 (s, 3H), 1.93-1.86 (m, 1H), 1.66-1.59 (m, 1H), 1.56-1.45 (m, 2H), 1.29 (s, 3H), 1.27 (s, 3H), 0.70 (s, 3H). A yield of the compound 3c was 44%.

Compound 3d:

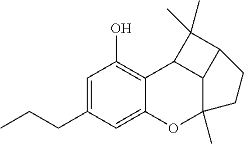 [SMILES: CCCc1cc(O)c2c(c1)OC1(C)CCC3C1C2C3(C)C]

(raw material including:

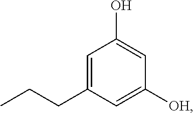

which was synthesized by the synthesis manner (2)). NMR data of the compound 3d includes: ¹H NMR (600 MHz, CDCl₃) δ 6.33 (s, 1H), 6.18 (s, 1H), 4.54 (s, 1H), 3.07 (d, J=9.7 Hz, 1H), 2.57 (t, J=7.9 Hz, 1H), 2.48-2.42 (m, 2H), 2.40 (t, J=7.3 Hz, 1H), 2.02-1.95 (m, 1H), 1.71-1.67 (m, 1H), 1.63-1.56 (m, 4H), 1.38 (s, 3H), 1.38 (s, 3H), 0.91 (t, J=7.4 Hz, 3H), 0.80 (s, 3H). A yield of the compound 3d was 26%.

Compound 3e:

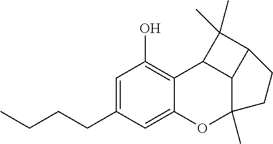 [SMILES: CCCCc1cc(O)c2c(c1)OC1(C)CCC3C1C2C3(C)C]

(raw material including

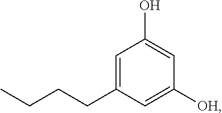

which was synthesized by the synthesis manner (2)). NMR data of the compound 3e includes: ¹H NMR (600 MHz, CDCl₃) δ 6.33 (s, 1H), 6.18 (s, 1H), 4.56 (s, 1H), 3.06 (d, J=9.7 Hz, 1H), 2.57 (t, J=7.9 Hz, 1H), 2.51-2.42 (m, 2H), 2.39 (t, J=7.4 Hz, 1H), 2.02-1.94 (m, 1H), 1.72-1.65 (m, 1H), 1.65-1.51 (m, 4H), 1.38 (s, 3H), 1.38 (s, 3H), 1.32 (q, J=7.4 Hz, 2H), 0.90 (t, J=7.4 Hz, 3H), 0.80 (s, 3H). A yield of the compound 3e was 30%.

Compound 3f:

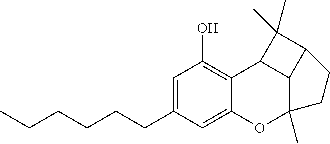 [SMILES: CCCCCCc1cc(O)c2c(c1)OC1(C)CCC3C1C2C3(C)C]

(raw material including:

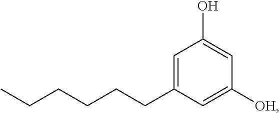

which is synthesized by the synthesis manner (2)). NMR data of the compound 3f includes: ¹H NMR (600 MHz, CDCl₃) δ 6.33 (s, 1H), 6.18 (s, 1H), 4.66 (s, 1H), 3.07 (d, J=9.6 Hz, 1H), 2.58 (t, J=8.5 Hz, 1H), 2.49-2.42 (m, 2H), 2.40 (t, J=7.4 Hz, 1H), 2.03-1.95 (m, 1H), 1.72-1.66 (m, 1H), 1.66-1.52 (m, 4H), 1.39 (s, 3H), 1.38 (s, 3H), 1.33-1.24 (m, 6H), 0.87 (t, J=6.6 Hz, 3H), 0.80 (s, 3H). A yield of the compound 3f was 29%.

Compound 3g:

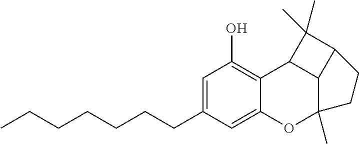 [SMILES: CCCCCCCc1cc(O)c2c(c1)OC1(C)CCC3C1C2C3(C)C]

(raw material including:

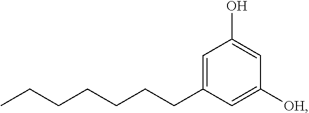

which was synthesized by the synthesis manner (2)). NMR data of the compound 3g includes: ¹H NMR (600 MHz, CDCl₃) δ 6.32 (s, 1H), 6.17 (s, 1H), 4.56 (s, 1H), 3.06 (d, J=9.6 Hz, 1H), 2.57 (t, J=7.7 Hz, 1H), 2.50-2.42 (m, 2H), 2.39 (t, J=7.4 Hz, 1H), 2.01-1.94 (m, 1H), 1.72-1.67 (m, 1H), 1.65-1.52 (m, 4H), 1.38 (s, 3H), 1.38 (s, 3H), 1.31-1.24 (m, 8H), 0.87 (t, J=6.8 Hz, 3H), 0.80 (s, 3H). A yield of the compound 3g was 22%.

Compound 3h:

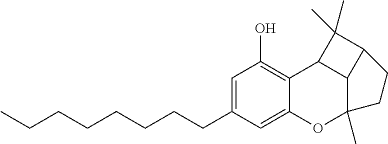 [SMILES: CCCCCCCCc1cc(O)c2c(c1)OC1(C)CCC3C1C2C3(C)C]

(raw material including:

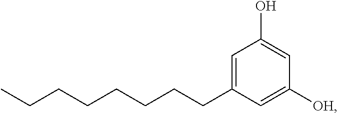 [SMILES: CCCCCCCCc1cc(O)cc(O)c1]

which was synthesized by the synthesis manner (2)). NMR data of the compound 3h includes: ¹H NMR (600 MHz, CDCl₃) δ 6.32 (s, 1H), 6.18 (s, 1H), 4.50 (s, 1H), 3.06 (d, J=9.6 Hz, 1H), 2.57 (t, J=8.4 Hz, 1H), 2.51-2.42 (m, 2H), 2.39 (t, J=7.4 Hz, 1H), 2.01-1.94 (m, 1H), 1.70-1.66 (m, 1H), 1.65-1.53 (m, 4H), 1.38 (s, 3H), 1.37 (s, 3H), 1.30-1.22 (m, 10H), 0.87 (t, J=6.9 Hz, 3H), 0.80 (s, 3H). A yield of the compound 3h was 27%.

Compound 3i:

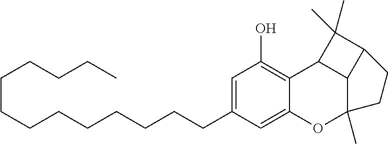 [SMILES: CCCCCCCCCCCCCc1cc(O)c2c(c1)OC1(C)CCC3C1C2C3(C)C]

(raw material including:

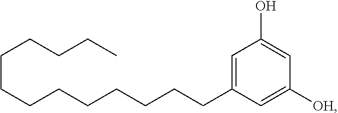 [SMILES: CCCCCCCCCCCCCc1cc(O)cc(O)c1]

which was synthesized by the synthesis manner (2)). NMR data of the compound 3i includes: ¹H NMR (600 MHz, CDCl₃) δ 6.32 (s, 1H), 6.17 (s, 1H), 4.54 (s, 1H), 3.06 (d, J=9.6 Hz, 1H), 2.57 (t, J=8.4 Hz, 1H), 2.48-2.43 (m, 2H), 2.39 (t, J=7.4 Hz, 1H), 2.01-1.94 (m, 1H), 1.71-1.67 (m, 1H), 1.64-1.52 (m, 4H), 1.38 (s, 3H), 1.38 (s, 3H), 1.30-1.23 (m, 20H), 0.88 (t, J=6.9 Hz, 3H), 0.80 (s, 3H). A yield of the compound 3i was 20%.

Compound 3j:

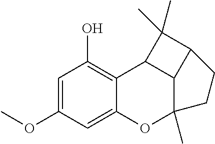 [SMILES: COc1cc(O)c2c(c1)OC1(C)CCC3C1C2C3(C)C]

(raw material including

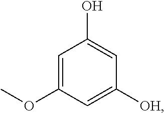

which was synthesized by the synthesis manner (1)). NMR data of the compound 3j includes: ¹H NMR (600 MHz, CDCl₃) δ 6.07 (s, 1H), 5.97 (s, 1H), 4.80 (s, 1H), 3.72 (s, 3H), 3.03 (d, J=9.7 Hz, 1H), 2.57 (t, J=8.2 Hz, 1H), 2.39 (t, J=7.5 Hz, 1H), 2.01-1.94 (m, 1H), 1.71-1.54 (m, 3H), 1.38 (s, 3H), 1.36 (s, 3H), 0.78 (s, 3H). A yield of the compound 3j was 28%.

Compound 3k:

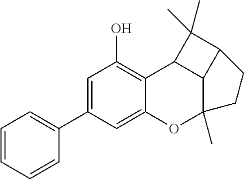 [SMILES: CC12CCC3C1C(c1c(O)cc(-c4ccccc4)cc1O2)C3(C)C]

(raw material including:

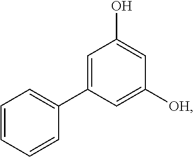 [SMILES: Oc1cc(O)cc(-c2ccccc2)c1]

which was synthesized by the synthesis manner (1)). NMR data of the compound 3k includes: ¹H NMR (600 MHz, CDCl₃) δ 7.55 (d, J=7.1 Hz, 2H), 7.39 (t, J=7.6 Hz, 2H), 7.31 (t, J=7.4 Hz, 1H), 6.77 (s, 1H), 6.59 (s, 1H), 4.69 (s, 1H), 3.15 (d, J=9.6 Hz, 1H), 2.63 (t, J=8.3 Hz, 1H), 2.43 (t, J=7.4 Hz, 1H), 2.07-2.00 (m, 1H), 1.75-1.56 (m, 3H), 1.42 (s, 3H), 1.41 (s, 3H), 0.85 (s, 3H). A yield of the compound 3k was 47%.

Compound 3l:

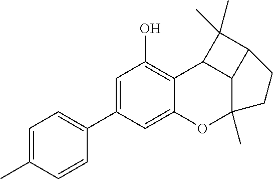 [SMILES: Cc1ccc(-c2cc(O)c3c(c2)OC2(C)CCC4C2C3C4(C)C)cc1]

(raw material including:

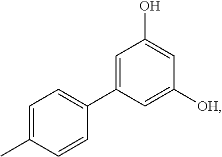

which was synthesized by the synthesis manner (1)). NMR data of the compound 31 includes: ¹H NMR (600 MHz, CDCl₃) δ 7.45 (d, J=7.8 Hz, 2H), 7.20 (d, J=7.8 Hz, 2H), 6.75 (s, 1H), 6.58 (s, 1H), 4.68 (s, 1H), 3.14 (d, J=9.6 Hz, 1H), 2.62 (t, J=8.4 Hz, 1H), 2.43 (t, J=7.4 Hz, 1H), 2.37 (s, 3H), 2.08-1.99 (m, 1H), 1.74-1.57 (m, 3H), 1.42 (s, 3H), 1.41 (s, 3H), 0.85 (s, 3H). A yield of the compound 3l was 35%.

Compound 3m:

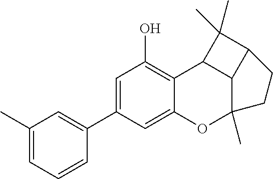 [SMILES: Cc1cccc(-c2cc(O)c3c(c2)OC2(C)CCC4C2C3C4(C)C)c1]

(raw material including

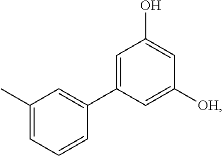

which was synthesized by the synthesis manner (1)). NMR data of the compound 3m includes: ¹H NMR (600 MHz, CDCl₃) δ 7.37 (s, 1H), 7.35 (d, J=7.9 Hz, 1H), 7.29-7.25 (m, 1H), 7.12 (d, J=7.5 Hz, 1H), 6.75 (s, 1H), 6.58 (s, 1H), 4.71 (s, 1H), 3.14 (d, J=9.6 Hz, 1H), 2.62 (t, J=8.1 Hz, 1H), 2.42 (t, J=7.5 Hz, 1H), 2.38 (s, 3H), 2.04-1.98 (m, 1H), 1.74-1.57 (m, 3H), 1.41 (s, 3H), 1.40 (s, 3H), 0.84 (s, 3H). A yield of the compound 3m was 35%.

Compound 3n:

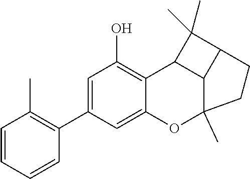 [SMILES: Cc1ccccc1-c1cc(O)c2c(c1)OC1(C)CCC3C1C2C3(C)C]

(raw material including:

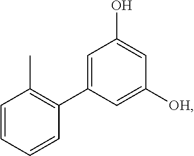

which was synthesized by the synthesis manner (1)). NMR data of the compound 3n includes: ¹H NMR (600 MHz, CDCl₃) δ 7.24-7.17 (m, 4H), 6.46 (s, 1H), 6.29 (s, 1H), 4.63 (s, 1H), 3.14 (d, J=9.6 Hz, 1H), 2.62 (t, J=8.0 Hz, 1H), 2.43 (t, J=7.4 Hz, 1H), 2.29 (s, 3H), 2.07-2.00 (m, 1H), 1.77-1.69 (m, 1H), 1.67-1.59 (m, 2H), 1.41 (s, 6H), 0.86 (s, 3H). A yield of the compound 3n was 24%.

Compound 3o:

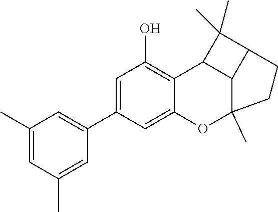 [SMILES: Cc1cc(C)cc(-c2cc(O)c3c(c2)OC2(C)CCC4C2C3C4(C)C)c1]

(raw material including:

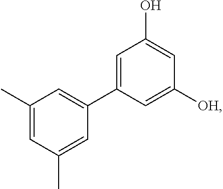 [SMILES: Cc1cc(C)cc(-c2cc(O)cc(O)c2)c1]

which was synthesized by the synthesis manner (1)). NMR data of the compound 3o includes: ¹H NMR (600 MHz, CDCl₃) δ 7.18 (s, 2H), 6.96 (s, 1H), 6.75 (s, 1H), 6.58 (s, 1H), 4.74 (s, 1H), 3.14 (d, J=9.6 Hz, 1H), 2.62 (t, J=8.2 Hz, 1H), 2.43 (t, J=7.4 Hz, 1H), 2.35 (s, 6H), 2.05-1.98 (m, 1H), 1.75-1.57 (m, 3H), 1.42 (s, 3H), 1.41 (s, 3H), 0.84 (s, 3H). A yield of the compound 3o was 36%.

Compound 3p:

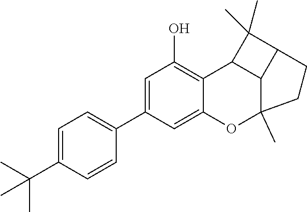 [SMILES: CC(C)(C)c1ccc(-c2cc(O)c3c(c2)OC2(C)CCC4C2C3C4(C)C)cc1]

(raw material including:

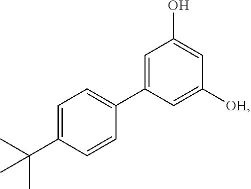 [SMILES: CC(C)(C)c1ccc(-c2cc(O)cc(O)c2)cc1]

which was synthesized by the synthesis manner (1)). NMR data of the compound 3p includes: ¹H NMR (500 MHz, CDCl₃) δ 7.47 (d, J=8.4 Hz, 2H), 7.42-7.37 (m, 2H), 6.62 (s, 1H), 6.58 (s, 1H), 3.16 (d, J=9.6 Hz, 1H), 2.55 (t, J=8.7 Hz, 1H), 2.38 (t, J=7.4 Hz, 1H), 2.06-1.97 (m, 1H), 1.72-1.54 (m, 3H), 1.38 (s, 3H), 1.35 (s, 3H), 1.33 (d, J=27.4 Hz, 9H), 0.81 (s, 3H). A yield of the compound 3p was 23%.

Compound 3q:

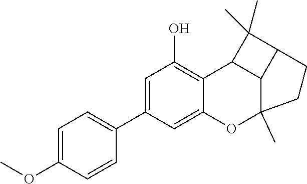 [SMILES: COc1ccc(-c2cc(O)c3c(c2)OC2(C)CCC4C2C3C4(C)C)cc1]

(raw material including:

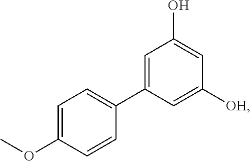 [SMILES: COc1ccc(-c2cc(O)cc(O)c2)cc1]

which was synthesized by the synthesis manner (1)). NMR data of the compound 3q includes: ¹H NMR (600 MHz, CDCl₃) δ 7.49 (d, J=7.9 Hz, 2H), 7.41 (d, J=7.8 Hz, 2H), 6.76 (s, 1H), 6.58 (s, 1H), 4.66 (s, 1H), 3.14 (d, J=9.5 Hz, 1H), 2.62 (t, J=8.6 Hz, 1H), 2.43 (t, J=7.6 Hz, 1H), 2.07-1.98 (m, 1H), 1.75-1.69 (m, 1H), 1.68-1.60 (m, 2H), 1.41 (d, J=5.4 Hz, 6H), 1.35 (s, 9H), 0.84 (s, 3H). A yield of the compound 3q was 36%.

Compound 3r:

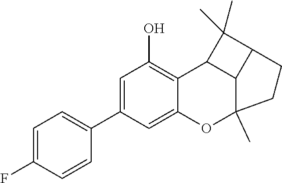 [SMILES: CC12CCC3C1C(c1c(O)cc(-c4ccc(F)cc4)cc1O2)C3(C)C]

(raw material including:

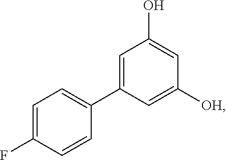 [SMILES: Oc1cc(O)cc(-c2ccc(F)cc2)c1]

which was synthesized by the synthesis manner (1)). NMR data of the compound 3r includes: ¹H NMR (600 MHz, CDCl₃) δ 7.49 (t, J=7.0 Hz, 2H), 7.07 (t, J=8.6 Hz, 2H), 6.70 (d, J=1.7 Hz, 1H), 6.53 (d, J=1.6 Hz, 1H), 4.73 (d, J=4.5 Hz, 1H), 3.13 (d, J=9.6 Hz, 1H), 2.62 (t, J=8.0 Hz, 1H), 2.43 (t, J=7.4 Hz, 1H), 2.02 (t, J=11.1, 6.3 Hz, 1H), 1.74-1.57 (m, 3H), 1.42 (s, 3H), 1.41 (s, 3H), 0.84 (s, 3H). A yield of the compound 3r was 43%.

Compound 3s:

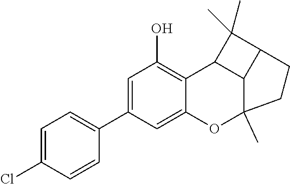 [SMILES: CC12CCC3C1C(c1c(O)cc(-c4ccc(Cl)cc4)cc1O2)C3(C)C]

(raw material including:

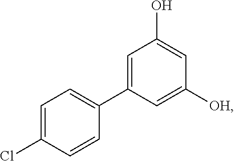 [SMILES: Oc1cc(O)cc(-c2ccc(Cl)cc2)c1]

which was synthesized by the synthesis manner (1)). NMR data of the compound 3s includes: ¹H NMR (600 MHz, CDCl₃) δ 7.43 (d, J=8.3 Hz, 2H), 7.35 (d, J=8.2 Hz, 2H), 6.68 (d, J=10.1 Hz, 1H), 6.64 (s, 1H), 6.48 (s, 1H), 5.59 (d, J=10.0 Hz, 1H), 5.17 (s, 1H), 5.10 (t, J=7.4 Hz, 1H), 2.18-2.06 (m, 2H), 1.81-1.74 (m, 1H), 1.71-1.67 (m, 1H), 1.67 (s, 3H), 1.58 (s, 3H), 1.42 (s, 3H). A yield of the compound 3s was 36%.

Compound 3t:

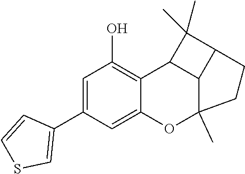 [SMILES: CC12CCC3C1C(c1c(O)cc(-c4ccsc4)cc1O2)C3(C)C]

(raw material including:

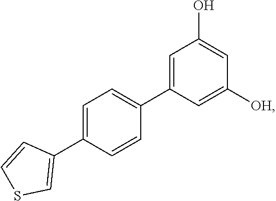 [SMILES: Oc1cc(O)cc(-c2ccc(-c3ccsc3)cc2)c1]

which was synthesized by the synthesis manner (1)). NMR data of the compound 3t includes: ¹H NMR (600 MHz, CDCl₃) δ 7.38 (t, J=2.0 Hz, 1H), 7.33 (d, J=2.5 Hz, 2H), 6.76 (s, 1H), 6.60 (s, 1H), 4.66 (s, 1H), 3.12 (d, J=9.6 Hz, 1H), 2.62 (t, J=8.3 Hz, 1H), 2.43 (t, J=7.5 Hz, 1H), 2.04-1.98 (m, 1H), 1.72-1.58 (m, 3H), 1.41 (s, 3H), 1.40 (s, 3H), 0.83 (s, 3H). A yield of the compound 3t was 22%.

### Example 2

### Synthesis of C-12 Modified Cannabicyclol Derivatives

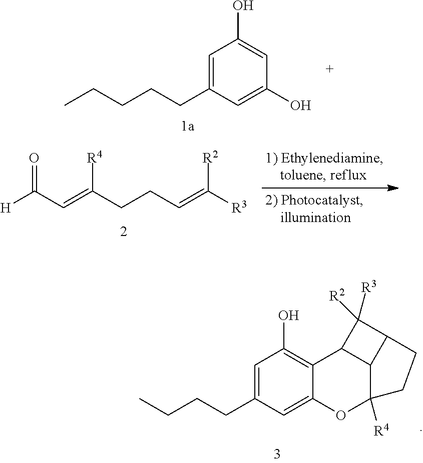

A synthesis procedure includes: taking a compound 3u of a formula

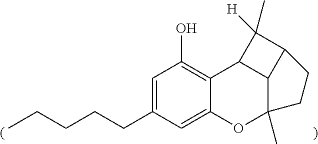 [SMILES: CCCCCc1cc(O)c2c(c1)OC1(C)CCC3C(C)C2C31]

as an example, olive alcohol 1a (36 mg, 0.2 mmol, 1.0 equiv.), citral derivative 2 (R=H, 27.6 mg, 0.2 mmol, 1.0 equiv.), and ethylenediamine (1 μL, 10 mol %, 0.01 equiv.) were added to 2 mL of toluene to obtain a reaction mixture. The reaction mixture was heated to reflux and reacted for 3 hours. Under a nitrogen atmosphere, after confirming completion of the reaction by TLC, the reaction mixture was stirred at room temperature. Then, [Ir(dF(CF₃)ppy)₂(bpy)]PF₆ (2 mg, 2 mol ‰, 0.005 equiv.) was added to the reaction mixture. The reaction mixture was irradiated with a 405 nm LED light source for 3 hours (alternatively, the reaction mixture was irradiated by sunlight for 5 days). After confirming the completion of the reaction by TLC, the reaction mixture was extracted with 25 mL of ethyl acetate three times, and the organic phase was collected. After concentrating the organic phase through rotary evaporation, the organic phase was purified by silica gel column chromatography, yielding 9.2 mg of a colorless oily liquid (yield 15%), i.e., the compound 3u. NMR data of the compound 3u includes: ¹H NMR (600 MHz, CDCl₃) δ 6.33 (s, 1H), 6.18 (s, 1H), 4.56 (s, 1H), 2.90 (t, J=7.7 Hz, 1H), 2.51-2.40 (m, 3H), 2.32 (q, J=6.2 Hz, 1H), 2.17-2.08 (m, 1H), 1.81 (q, J=6.4 Hz, 1H), 1.72-1.60 (m, 2H), 1.56 (p, J=7.5 Hz, 2H), 1.48 (dd, J=12.8, 7.2 Hz, 1H), 1.38 (s, 3H), 1.35 (d, J=7.0 Hz, 3H), 1.33-1.25 (m, 4H), 0.88 (t, J=6.8 Hz, 3H). A yield of the compound 3u was 15%.

Using the synthesis method, a series of C-12 modified cannabicyclol derivatives may be synthesized, including the following cannabicyclol derivatives.

Compound 3v:

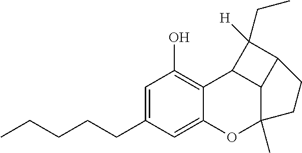 [SMILES: CCCCCc1cc(O)c2c(c1)OC1(C)CCC3C(CC)C2C31]

(raw material including

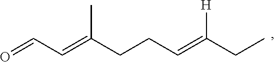

which was synthesized by the synthesis manner (3)). NMR data of the compound 3v includes: ¹H NMR (600 MHz, CDCl₃) δ 6.33 (s, 1H), 6.19 (s, 1H), 4.51 (s, 1H), 2.95 (t, J=7.7 Hz, 1H), 2.47-2.43 (m, 3H), 2.39 (q, J=6.5 Hz, 1H), 2.18-2.11 (m, 1H), 1.99-1.90 (m, 1H), 1.78-1.69 (m, 1H), 1.67-1.62 (m, 2H), 1.63-1.52 (m, 3H), 1.49-1.41 (m, 1H), 1.38 (s, 3H), 1.34-1.24 (m, 4H), 0.87 (t, J=7.1 Hz, 6H). A yield of the compound 3v was 11%.

Compound 3w:

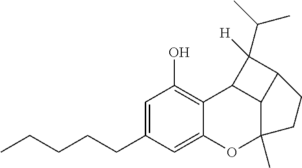 [SMILES: CCCCCc1cc(O)c2c(c1)OC1(C)CCC3C(C(C)C)C2C31]

(raw material including

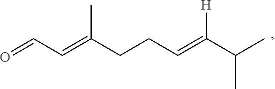

which was synthesized by the synthesis manner (3)). NMR data of the compound 3w includes: ¹H NMR (600 MHz, CDCl₃) δ 6.35 (s, 1H), 6.19 (s, 1H), 4.57 (s, 1H), 3.15 (t, J=8.0 Hz, 1H), 2.50-2.43 (m, 3H), 2.39 (t, J=8.4 Hz, 1H), 2.22-2.15 (m, 1H), 1.94-1.87 (m, 1H), 1.86-1.80 (m, 1H), 1.69-1.62 (m, 2H), 1.56 (t, J=7.5 Hz, 2H), 1.51-1.45 (m, 1H), 1.31 (s, 3H), 1.33-1.27 (m, 4H), 0.97 (d, J=6.7 Hz, 3H), 0.88 (dd, J=6.8, 3.6 Hz, 6H). A yield rate of the compound 3w was 14%.

Compound 3x:

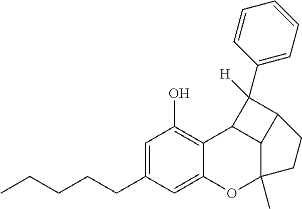 [SMILES: CCCCCc1cc(O)c2c(c1)OC1(C)CCC3C(c4ccccc4)C2C31]

(raw material including

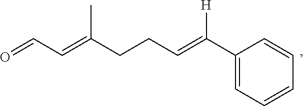

which was synthesized by the synthesis manner (3)). NMR data of the compound 3x includes: ¹H NMR (600 MHz, CDCl₃) δ 7.41 (d, J=7.5 Hz, 2H), 7.36 (t, J=7.5 Hz, 2H), 7.24 (d, J=7.1 Hz, 1H), 6.35 (s, 1H), 6.20 (s, 1H), 4.06 (s, 1H), 3.37 (t, J=7.7 Hz, 1H), 2.91-2.85 (m, 2H), 2.69-2.65 (m, 1H), 2.47-2.43 (m, 2H), 2.31-2.24 (m, 1H), 1.82-1.70 (m, 2H), 1.64 (dd, J=12.7, 7.1 Hz, 1H), 1.55 (p, J=7.6 Hz, 2H), 1.44 (s, 3H), 1.33-1.25 (m, 4H), 0.86 (t, J=6.8 Hz, 3H). A yield of the compound 3x was 15%.

Compound 3y:

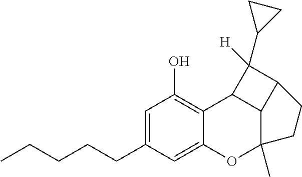 [SMILES: CCCCCc1cc(O)c2c(c1)OC1(C)CCC3C(C4CC4)C2C31]

(raw material including

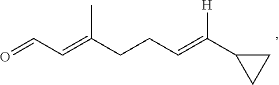

which was synthesized by the synthesis manner (3)). NMR data of the compound 3y includes: ¹H NMR (600 MHz, CDCl₃) δ 6.32 (s, 1H), 6.29 (s, 1H), 4.86 (s, 1H), 3.11-3.06 (m, 1H), 2.59-2.55 (m, 1H), 2.50-2.44 (m, 3H), 2.10-2.02 (m, 1H), 1.76-1.68 (m, 1H), 1.66-1.62 (m, 1H), 1.57 (p, J=7.6 Hz, 2H), 1.43-1.40 (m, 1H), 1.39 (s, 3H), 1.33-1.27 (m, 4H), 1.15-1.13 (m, 2H), 0.88 (t, J=6.8 Hz, 3H), 0.67-0.53 (m, 2H), 0.24-0.13 (m, 2H). A yield of the compound 3y was 12%.

Compound 3z:

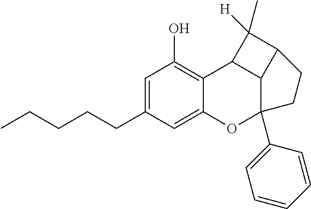 [SMILES: CCCCCc1cc(O)c2c(c1)OC1(c3ccccc3)CCC3C(C)C2C31]

(raw material including

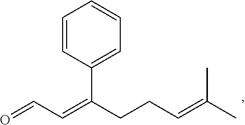 [SMILES: CC(C)=CCC/C(=C/C=O)c1ccccc1]

which was synthesized by the synthesis manner (4)). NMR data of the compound 3z includes: ¹H NMR (600 MHz, CDCl₃) δ 7.50 (d, J=7.7 Hz, 2H), 7.37 (t, J=7.6 Hz, 2H), 7.28 (d, J=7.4 Hz, 1H), 6.50 (s, 1H), 6.23 (s, 1H), 4.60 (s, 1H), 3.28 (d, J=9.8 Hz, 1H), 3.20 (t, J=8.9 Hz, 1H), 2.63 (t, J=7.8 Hz, 1H), 2.51 (t, J=7.8 Hz, 2H), 2.41-2.35 (m, 1H), 2.02-1.96 (m, 1H), 1.88-1.77 (m, 2H), 1.62 (p, J=7.5 Hz, 2H), 1.47 (s, 3H), 1.40-1.29 (m, 4H), 0.92 (t, J=6.9 Hz, 3H), 0.85 (s, 3H). A yield of the compound 3z was 24%.

### Example 3

### Synthesis of Multi-Substituted Cannabicyclol Derivative

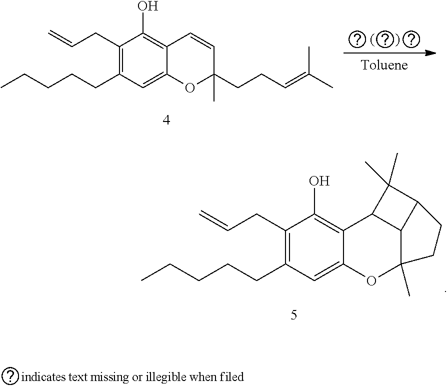

A synthesis procedure includes: cannabinophene derivative 4 (35 mg, 0.1 mmol, 1.0 equiv.), 2,3-dichloro-5,6-dicyanobenzoquinone (DDQ, 2.6 mg, 10 mol %), and indium trifluoromethanesulfonate (In(OTf)3, 5.7 mg, 10 mol %) were added to 1 mL of toluene under a nitrogen atmosphere to obtain a reaction mixture. After confirming the completion of a reaction by TLC, the reaction mixture was passed through a short silica gel column, concentrated through rotary evaporation, and purified by silica gel column chromatography to yield 12 mg of a colorless oily liquid (34% yield), i.e., a compound 5. NMR data of the compound 5 includes: ¹H NMR (600 MHz, CDCl₃) δ 6.19 (s, 1H), 5.94-5.85 (m, 1H), 4.99-4.88 (m, 2H), 4.35 (s, 1H), 3.43-3.23 (m, 2H), 3.07 (d, J=9.6 Hz, 1H), 2.59-2.51 (m, 2H), 2.50-2.41 (m, 1H), 2.37 (t, J=7.4 Hz, 1H), 1.96-1.88 (m, 1H), 1.69-1.63 (m, 1H), 1.60-1.49 (m, 4H), 1.37 (s, 6H), 1.35-1.32 (m, 4H), 0.89 (t, J=6.8 Hz, 3H), 0.77 (s, 3H).

### Example 4

### Synthesis of 5-OH Modified Cannabicyclol Derivatives

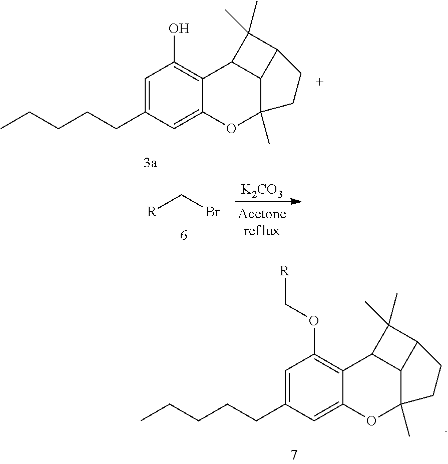

Taking a compound 7a of a formula

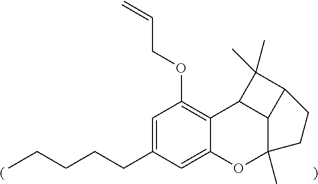 [SMILES: C=CCOc1cc(CCCCC)cc2c1C1C3C(CCC3(C)O2)C1(C)C]

as an example, after synthesizing a cannabicyclol derivative 3a according to Example 1, the compound 3a (31.4 mg, 0.1 mmol, 1.0 equiv.), allyl bromide 6 (24.2 mg, 0.2 mmol, 2.0 equiv.), and potassium carbonate (27.6 mg, 0.2 mmol, 2.0 equiv.) were added to 2 mL of acetone under a nitrogen atmosphere to obtain a reaction mixture, and the reaction mixture was reacted at 70° C. After confirming the completion of the reaction by TLC, the reaction mixture was passed through a short silica gel column, concentrated through rotary evaporation, and purified by silica gel column chromatography to yield 25.3 mg of a colorless oily liquid (71% yield), i.e., the compound 7a. NMR data of the compound 7a includes: ¹H NMR (600 MHz, CDCl₃) δ 6.35 (d, J=16.7 Hz, 1H), 6.22 (s, 1H), 6.14-6.01 (m, 1H), 5.38 (d, J=17.2 Hz, 1H), 5.26 (d, J=10.4 Hz, 1H), 4.51-4.42 (m, 2H), 3.10 (d, J=9.6 Hz, 1H), 2.56-2.45 (m, 3H), 2.36 (t, J=7.4 Hz, 1H), 2.00-1.91 (m, 1H), 1.70-1.63 (m, 1H), 1.62-1.53 (m, 4H), 1.37 (s, 3H), 1.34 (s, 3H), 1.32-1.28 (m, 4H), 0.88 (t, J=6.7 Hz, 3H), 0.73 (s, 3H).

Using the above synthesis method, a series of 5-OH modified cannabicyclol derivatives were synthesized, including the following cannabicyclol derivatives.

Compound 7b:

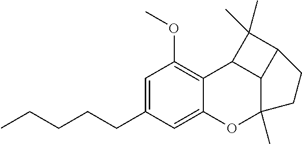 [SMILES: CCCCCc1cc(OC)c2c(c1)OC1(C)CCC3C1C2C3(C)C]

(raw material including: the compound 3a and methyl iodide). NMR data of the compound 7b includes: ¹H NMR (600 MHz, CDCl₃) δ 6.35 (s, 1H), 6.24 (s, 1H), 3.75 (s, 3H), 3.06 (d, J=9.6 Hz, 1H), 2.56-2.47 (m, 3H), 2.36 (t, J=7.4 Hz, 1H), 2.00-1.92 (m, 1H), 1.68-1.64 (m, 1H), 1.62-1.54 (m, 4H), 1.38 (s, 3H), 1.34 (s, 3H), 1.34-1.28 (m, 4H), 0.89 (t, J=6.8 Hz, 3H), 0.71 (s, 3H). A yield of the compound 7b was 73%.

Compound 7c:

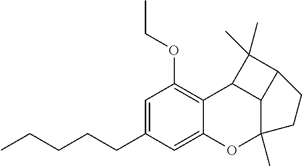 [SMILES: CCCCCc1cc(OCC)c2c(c1)OC1(C)CCC3C1C2C3(C)C]

(raw material including: the compound 3a and ethyl iodide). NMR data of the compound 7c includes: ¹H NMR (600 MHz, CDCl₃) δ 6.33 (s, 1H), 6.21 (s, 1H), 3.99-3.92 (m, 2H), 3.07 (d, J=9.6 Hz, 1H), 2.57-2.45 (m, 3H), 2.36 (t, J=7.3 Hz, 1H), 2.02-1.92 (m, 1H), 1.69-1.63 (m, 1H), 1.62-1.55 (m, 4H), 1.40 (t, J=7.0 Hz, 3H), 1.38 (s, 3H), 1.35 (s, 3H), 1.33-1.28 (m, 4H), 0.88 (t, J=6.7 Hz, 3H), 0.74 (s, 3H). A yield of the compound 7c was 57%.

Compound 7d:

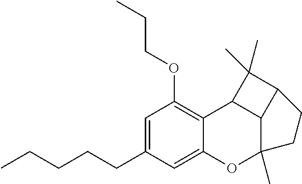 [SMILES: CCCCCc1cc(OCCC)c2c(c1)OC1(C)CCC3C1C2C3(C)C]

(raw material including the compound 3a and 1-Iodopropane). NMR data of the compound 7d includes: ¹H NMR (600 MHz, CDCl₃) δ 6.34 (s, 1H), 6.22 (s, 1H), 3.93-3.87 (m, 1H), 3.82-3.78 (m, 1H), 3.09 (d, J=9.6 Hz, 1H), 2.55-2.47 (m, 3H), 2.36 (t, J=7.3 Hz, 1H), 2.01-1.94 (m, 1H), 1.86-1.76 (m, 2H), 1.69-1.64 (m, 1H), 1.63-1.56 (m, 4H), 1.38 (s, 3H), 1.35 (s, 3H), 1.33-1.30 (m, 4H), 1.05 (t, J=7.4 Hz, 3H), 0.88 (t, J=6.7 Hz, 3H), 0.74 (s, 3H). A yield of the compound 7d was 48%.

Compound 7e:

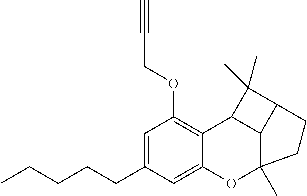 [SMILES: C#CCOc1cc(CCCCC)cc2c1C1C3C(CCC3(C)O2)C1(C)C]

(raw material including the compound 3a and 3-bromopropyne). NMR data of the compound 7e includes: ¹H NMR (600 MHz, CDCl₃) δ 6.38 (s, 1H), 6.28 (s, 1H), 4.66-4.58 (m, 2H), 3.09 (d, J=9.6 Hz, 1H), 2.57-2.47 (m, 4H), 2.37 (t, J=7.3 Hz, 1H), 2.00-1.91 (m, 1H), 1.69-1.64 (m, 1H), 1.61-1.55 (m, 4H), 1.38 (s, 3H), 1.37 (s, 3H), 1.34-1.29 (m, 4H), 0.88 (t, J=6.7 Hz, 3H), 0.74 (s, 3H). A yield of the compound 7e was 20%.

Compound 7f:

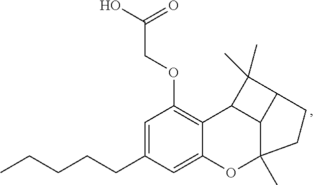 [SMILES: CCCCCc1cc(OCC(=O)O)c2c(c1)OC1(C)CCC3C1C2C3(C)C]

and a method for synthesizing the compound 7f includes: after synthesizing the cannabicyclol derivative 3a according to Example 1, under a nitrogen atmosphere, the compound 3a (94.2 mg, 0.3 mmol, 1.0 equiv.), ethyl bromoacetate (50.1 mg, 0.3 mmol, 1.0 equiv.), potassium iodide (50 mg, 0.3 mmol, 1.0 equiv.), and potassium carbonate (82.9 mg, 0.6 mmol, 2.0 equiv.) were added to 10 mL of acetone and heated at 70° C. for 6 hours to obtain a reaction mixture. After confirming the completion of the reaction by TLC, the reaction mixture was passed through a short silica gel column, concentrated through rotary evaporation, and purified by silica gel column chromatography to yield a purified compound. The purified compound (84.2 mg, 0.2 mmol, 1.0 equiv.) and the potassium carbonate (82.8 mg, 0.6 mmol, 3.0 equiv.) were added to 5 mL of methanol to obtain a reaction mixture. After confirming the completion of the reaction by TLC, the reaction mixture was passed through a short silica gel column, concentrated through rotary evaporation, and purified by silica gel column chromatography to yield 25 mg of a colorless oily liquid (combined yield of 23%), i.e., the compound 7f. NMR data of the compound 7f includes: ¹H NMR (600 MHz, CDCl₃) δ 6.42 (s, 1H), 6.12 (s, 1H), 4.62 (d, J=4.3 Hz, 2H), 3.15 (d, J=9.6 Hz, 1H), 2.55 (t, J=8.3 Hz, 1H), 2.50 (q, J=7.5 Hz, 2H), 2.38 (t, J=7.3 Hz, 1H), 2.00-1.94 (m, 1H), 1.70-1.64 (m, 1H), 1.61-1.54 (m, 4H), 1.39 (s, 3H), 1.38 (s, 3H), 1.33-1.27 (m, 4H), 0.88 (t, J=6.8 Hz, 3H), 0.75 (s, 3H).

Compound 7g:

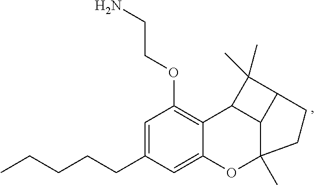 [SMILES: CCCCCc1cc(OCCN)c2c(c1)OC1(C)CCC3C1C2C3(C)C]

and a method for synthesizing the compound 7g includes: after synthesizing the cannabicyclol derivative 3a according to Example 1, under a nitrogen atmosphere, the compound 3a (94.2 mg, 0.3 mmol, 1.0 equiv.), 2-(Boc-amino)ethyl bromide (67.2 mg, 0.3 mmol, 1.0 equiv.), potassium iodide (50 mg, 0.3 mmol, 1.0 equiv.), potassium carbonate (82.9 mg, 0.6 mmol, 2.0 equiv.) were added to 10 mL of acetone and heated and stirred at 70° C. to obtain a reaction mixture. After confirming the completion of the reaction by TLC, the reaction mixture was passed through a short silica gel column, concentrated through rotary evaporation, and purified by silica gel column chromatography to yield a purified compound. The purified compound (92 mg, 0.2 mmol, 1.0 equiv.) and trifluoroacetic acid (3.42 mg, 15 mol %, 0.03 equiv.) were added to 5 mL of dichloromethane to obtain a reaction mixture. After confirming the completion of the reaction by TLC, the pH of the reaction mixture was adjusted to 8 to 9 by adding 2M NaOH solution, and the reaction mixture was extracted with 25 mL of ethyl acetate three times, and the organic phase was collected. After concentrating the organic phase through rotary evaporation, the organic phase was purified by silica gel column chromatography, yielding 48 mg of a pale yellow oily liquid (yield 67%), i.e., the compound 7g. NMR data of the compound 7g includes: ¹H NMR (600 MHz, CDCl₃) δ 6.36 (s, 1H), 6.21 (s, 1H), 4.02-3.94 (m, 2H), 3.64 (s, 2H), 3.17-3.07 (m, 3H), 2.55-2.44 (m, 3H), 2.35 (t, J=7.3 Hz, 1H), 1.99-1.91 (m, 1H), 1.67-1.61 (m, 1H), 1.61-1.53 (m, 4H), 1.36 (s, 3H), 1.33 (s, 3H), 1.32-1.28 (m, 4H), 0.87 (t, J=6.8 Hz, 3H), 0.71 (s, 3H).

Compound 7h:

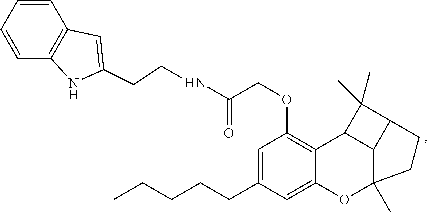 [SMILES: CCCCCc1cc(OCC(=O)NCCc2cc3ccccc3[nH]2)c2c(c1)OC1(C)CCC3C1C2C3(C)C]

and a method for synthesizing the compound 7h includes: under a nitrogen atmosphere, the compound 7f (380.5 mg, 1 mmol, 1.0 equiv.), tryptamine (160 mg, 1 mmol, 1.0 equiv.), 1-(3-Dimethylaminopropyl)-3-ethylcarbodiimide hydrochloride (EDCl, 229 mg, 1.2 mmol, 1.2 equiv.), 1-hydroxybenzotriazole (HOBT, 148 mg, 1.1 mmol, 1.1 equiv.), and triethylamine (350 L) were added to 5 mL of dichloromethane to obtain a reaction mixture. After confirming the completion of the reaction by TLC, the reaction mixture was passed through a short silica gel column, concentrated through rotary evaporation, and purified by silica gel column chromatography to yield 200 mg of a colorless oily liquid (39% yield), i.e., the compound 7h. NMR data of the compound 7h includes: ¹H NMR (600 MHz, CDCl₃) δ 7.99 (s, 1H), 7.56 (d, J=7.9 Hz, 1H), 7.35 (d, J=8.1 Hz, 1H), 7.19 (t, J=7.6 Hz, 1H), 7.09 (t, J=7.5 Hz, 1H), 6.64 (s, 1H), 6.49 (t, J=6.0 Hz, 1H), 6.44 (s, 1H), 6.19 (s, 1H), 4.52 (d, J=15.4 Hz, 1H), 4.42 (d, J=15.3 Hz, 1H), 3.86-3.78 (m, 1H), 3.56-3.49 (m, 1H), 3.04-2.97 (m, 1H), 2.92-2.85 (m, 1H), 2.71 (d, J=9.5 Hz, 1H), 2.52-2.48 (m, 2H), 2.45 (t, J=8.0 Hz, 1H), 2.27 (t, J=7.4 Hz, 1H), 1.94-1.86 (m, 1H), 1.64-1.53 (m, 5H), 1.40 (s, 3H), 1.34-1.27 (m, 4H), 1.06 (s, 3H), 0.88 (t, J=6.2 Hz, 3H), 0.54 (s, 3H).

Compound 7i:

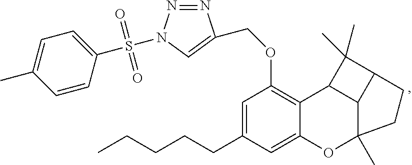 [SMILES: CCCCCc1cc(OCc2cn(S(=O)(=O)c3ccc(C)cc3)nn2)c2c(c1)OC1(C)CCC3C1C2C3(C)C]

and a method for synthesizing the compound 7i includes: under a nitrogen atmosphere, the compound 7e (70.4 mg, 0.2 mmol, 1.0 equiv.), para-toluenesulfonyl azide (59.1 mg, 0.3 mmol, 1.5 equiv.), and copper(I) thienyl-2-carboxylate (3.8 mg, 10 mol %, 0.1 equiv.) were added to 5 mL of toluene to obtain a reaction mixture. After confirming the completion of a reaction by TLC, the reaction mixture was passed through a short silica gel column, concentrated through rotary evaporation, and purified by silica gel column chromatography to yield 61.3 mg of a colorless oily liquid (77% yield), i.e., the compound 7i. NMR data of the compound 7i includes: ¹H NMR (600 MHz, CDCl₃) δ 8.16 (s, 1H), 7.98 (d, J=8.2 Hz, 2H), 7.38 (d, J=8.1 Hz, 2H), 6.39 (s, 1H), 6.28 (s, 1H), 5.16-5.09 (m, 2H), 3.02 (d, J=9.6 Hz, 1H), 2.55-2.47 (m, 3H), 2.45 (s, 3H), 2.32 (t, J=7.4 Hz, 1H), 2.00-1.89 (m, 1H), 1.66-1.60 (m, 1H), 1.59-1.52 (m, 4H), 1.37 (s, 3H), 1.34-1.26 (m, 4H), 1.07 (s, 3H), 0.88 (t, J=6.8 Hz, 3H), 0.64 (s, 3H). A yield of the compound 7i was 77%.

### Example 5

### Esterification of 5-OH Modified Cannabicyclol Derivative

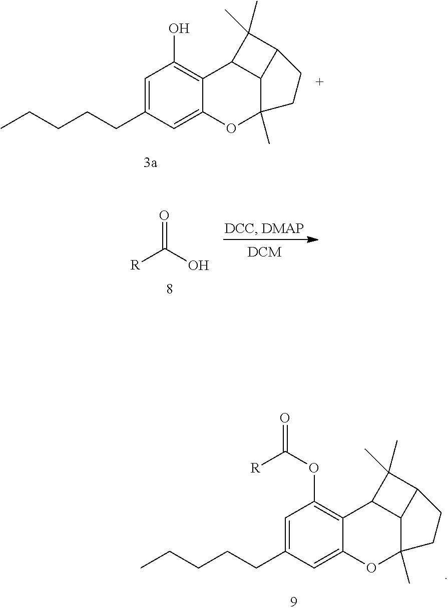

Taking a compound 9a:

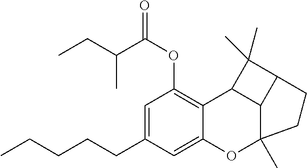 [SMILES: CCCCCc1cc(OC(=O)C(C)CC)c2c(c1)OC1(C)CCC3C1C2C3(C)C]

as an example, and a method for synthesizing the compound 9a includes: after synthesizing the cannabicyclol derivative 3a according to Example 1, the compound 3a (62.8 mg, 0.2 mmol, 1.0 equiv.), 2-Methylbutanoic acid (30.6 mg, 0.3 mmol, 1.5 equiv.), 4-Dimethylaminopyridine (DMAP, 5 mg, 20 mol %, 0.02 equiv.), and N,N′-di-cyclohexylcarbodiimide (DCC, 50 mg, 0.24 mmol, 1.2 equiv.) were added to 2 mL of dichloromethane to obtain a reaction mixture. After confirming the completion of a reaction by TLC, the reaction mixture was extracted with 25 mL of ethyl acetate three times, and the organic phase was collected. After concentrating the organic phase through rotary evaporation, the organic phase was purified by silica gel column chromatography, yielding 37 mg of a colorless oily liquid (46% yield), i.e., the compound 9a. NMR data of the compound 9a includes: ¹H NMR (600 MHz, CDCl₃) δ 6.60 (s, 1H), 6.45 (d, J=10.1 Hz, 1H), 2.99 (dd, J=15.2, 9.6 Hz, 1H), 2.64-2.57 (m, 1H), 2.56-2.48 (m, 3H), 2.39 (t, J=7.1 Hz, 1H), 2.02-1.93 (m, 1H), 1.88-1.81 (m, 1H), 1.73-1.67 (m, 1H), 1.65-1.55 (m, 5H), 1.34 (d, J=3.8 Hz, 3H), 1.33-1.29 (m, 8H), 1.27 (d, J=6.9 Hz, 2H), 1.06-1.00 (m, 3H), 0.88 (t, J=6.7 Hz, 3H), 0.72 (d, J=11.4 Hz, 3H).

Using the above synthesis method, substituted cannabicyclol derivatives were synthesized, including the following cannabicyclol derivatives.

Compound 9b:

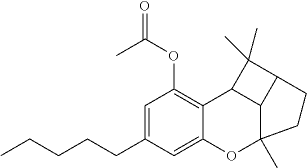 [SMILES: CCCCCc1cc(OC(C)=O)c2c(c1)OC1(C)CCC3C1C2C3(C)C]

(raw material including the compound 3a and acetic anhydride). NMR data of the compound 9b includes: ¹H NMR (600 MHz, CDCl₃) δ 6.60 (s, 1H), 6.48 (s, 1H), 2.97 (d, J=9.6 Hz, 1H), 2.56-2.48 (m, 3H), 2.38 (t, J=7.5 Hz, 1H), 2.29 (s, 3H), 2.00-1.91 (m, 1H), 1.72-1.67 (m, 1H), 1.65-1.55 (m, 4H), 1.36 (s, 3H), 1.32 (s, 3H), 1.31-1.27 (m, 4H), 0.88 (t, J=6.7 Hz, 3H), 0.75 (s, 3H). A yield of the compound 9b was 10%.

Compound 9c:

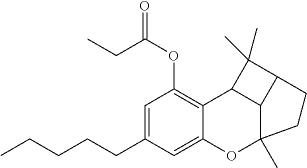 [SMILES: CCCCCc1cc(OC(=O)CC)c2c(c1)OC1(C)CCC3C1C2C3(C)C]

(raw material including the compound 3a and propionyl chloride). NMR data of the compound 9c includes: ¹H NMR (600 MHz, CDCl₃) δ 6.60 (s, 1H), 6.48 (s, 1H), 2.97 (d, J=9.6 Hz, 1H), 2.60-2.55 (m, 2H), 2.54-2.48 (m, 3H), 2.38 (t, J=7.1 Hz, 1H), 1.99-1.91 (m, 1H), 1.72-1.67 (m, 1H), 1.65-1.55 (m, 4H), 1.35 (s, 3H), 1.31 (s, 3H), 1.30-1.28 (m, 4H), 1.26 (t, J=7.5 Hz, 3H), 0.88 (t, J=6.8 Hz, 3H), 0.73 (s, 3H). A yield of the compound 9c was 31%.

Compound 9d:

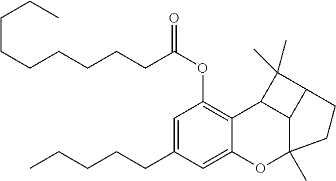 [SMILES: CCCCCCCCCC(=O)Oc1cc(CCCCC)cc2c1C1C3C(CCC3(C)O2)C1(C)C]

(raw material including the compound 3a and n-decanoic acid). NMR data of the compound 9d includes: ¹H NMR (600 MHz, CDCl₃) δ 6.59 (s, 1H), 6.47 (s, 1H), 2.97 (d, J=9.6 Hz, 1H), 2.58-2.48 (m, 5H), 2.38 (t, J=7.5 Hz, 1H), 1.99-1.93 (m, 1H), 1.74 (p, J=7.5 Hz, 2H), 1.72-1.67 (m, 1H), 1.64-1.55 (m, 4H), 1.43-1.37 (m, 2H), 1.35 (s, 3H), 1.31 (s, 3H), 1.31-1.25 (m, 14H), 0.90-0.86 (m, 6H), 0.74 (s, 3H). A yield of the compound 9d was 32%.

Compound 9e:

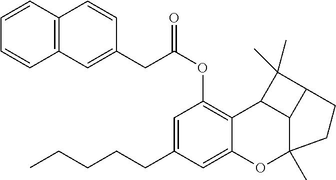 [SMILES: CCCCCc1cc(OC(=O)Cc2ccc3ccccc3c2)c2c(c1)OC1(C)CCC3C1C2C3(C)C]

(raw material including the compound 3a and 2-Naphthylacetic acid). NMR data of the compound 9e includes: ¹H NMR (600 MHz, CDCl₃) δ 7.91-7.80 (m, 4H), 7.54-7.47 (m, 3H), 6.60 (s, 1H), 6.45 (s, 1H), 4.03 (d, J=3.6 Hz, 2H), 3.00 (d, J=9.6 Hz, 1H), 2.53 (t, J=8.5 Hz, 1H), 2.49 (td, J=7.5, 2.8 Hz, 2H), 2.37 (t, J=7.2 Hz, 1H), 2.00-1.91 (m, 1H), 1.73-1.66 (m, 1H), 1.64-1.52 (m, 4H), 1.35 (s, 3H), 1.31-1.28 (m, 4H), 1.27 (s, 3H), 0.87 (t, J=6.8 Hz, 3H), 0.74 (s, 3H). A yield of the compound 9e was 31%.

Compound 9f:

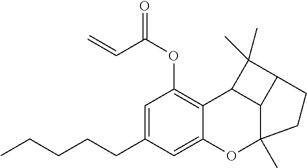 [SMILES: C=CC(=O)Oc1cc(CCCCC)cc2c1C1C3C(CCC3(C)O2)C1(C)C]

(raw material including the compound 3a and acryloyl chloride). NMR data of the compound 9f includes: ¹H NMR (600 MHz, CDCl₃) δ 6.63-6.57 (m, 2H), 6.52 (s, 1H), 6.32 (dd, J=17.3, 10.5 Hz, 1H), 6.01 (d, J=10.5 Hz, 1H), 2.99 (d, J=9.6 Hz, 1H), 2.56-2.49 (m, 3H), 2.37 (t, J=7.5 Hz, 1H), 2.00-1.93 (m, 1H), 1.72-1.67 (m, 1H), 1.64-1.56 (m, 4H), 1.36 (s, 3H), 1.31 (t, J=6.6 Hz, 4H), 1.26 (s, 3H), 0.91-0.86 (m, 3H), 0.75 (s, 3H).. A yield of the compound 9f was 46%.

Compound 9g:

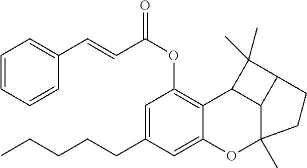 [SMILES: CCCCCc1cc(OC(=O)/C=C/c2ccccc2)c2c(c1)OC1(C)CCC3C1C2C3(C)C]

(raw material including the compound 3a and cinnamic acid). NMR data of the compound 9g includes: ¹H NMR (600 MHz, CDCl₃) δ 7.88 (d, J=16.0 Hz, 1H), 7.62-7.57 (m, 2H), 7.46-7.42 (m, 3H), 6.66-6.62 (m, 2H), 6.57 (s, 1H), 3.06 (d, J=9.6 Hz, 1H), 2.58-2.51 (m, 3H), 2.38 (t, J=7.5 Hz, 1H), 2.06-1.94 (m, 1H), 1.73-1.68 (m, 1H), 1.62 (hept, J=7.2, 6.1 Hz, 4H), 1.38 (s, 3H), 1.35-1.30 (m, 4H), 1.29 (s, 3H), 0.89 (t, J=6.3 Hz, 3H), 0.81 (s, 3H). A yield of the compound 9g was 38%.

Compound 9h:

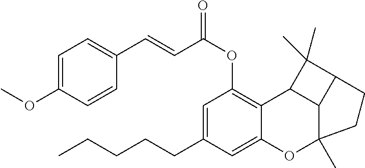 [SMILES: CCCCCc1cc(OC(=O)/C=C/c2ccc(OC)cc2)c2c(c1)OC1(C)CCC3C1C2C3(C)C]

(raw material including the compound 3a and trans-4-Methoxycinnamic acid). NMR data of the compound 9h includes: ¹H NMR (600 MHz, CDCl₃) δ 7.82 (d, J=15.9 Hz, 1H), 7.55 (d, J=8.4 Hz, 2H), 6.94 (d, J=8.3 Hz, 2H), 6.62 (s, 1H), 6.55 (s, 1H), 6.49 (d, J=15.9 Hz, 1H), 3.86 (s, 3H), 3.05 (d, J=9.6 Hz, 1H), 2.57-2.50 (m, 3H), 2.37 (t, J=7.5 Hz, 1H), 2.04-1.95 (m, 1H), 1.72-1.68 (m, 1H), 1.65-1.56 (m, 4H), 1.37 (s, 3H), 1.33-1.29 (m, 4H), 1.28 (s, 3H), 0.88 (t, J=6.3 Hz, 3H), 0.80 (s, 3H). A yield of the compound 9h was 57%.

Compound 9i:

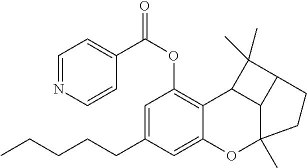 [SMILES: CCCCCc1cc(OC(=O)c2ccncc2)c2c(c1)OC1(C)CCC3C1C2C3(C)C]

(raw material including the compound 3a and isonicotinic acid). NMR data of the compound 9i includes: ¹H NMR (600 MHz, CDCl₃) δ 8.88 (d, J=4.9 Hz, 2H), 8.00 (d, J=5.1 Hz, 2H), 6.69 (s, 1H), 6.56 (s, 1H), 3.01 (d, J=9.6 Hz, 1H), 2.58-2.53 (m, 3H), 2.35 (t, J=7.6 Hz, 1H), 2.06-1.95 (m, 1H), 1.74-1.68 (m, 1H), 1.67-1.57 (m, 4H), 1.37 (s, 3H), 1.35-1.29 (m, 4H), 1.06 (s, 3H), 0.89 (t, J=6.7 Hz, 3H), 0.79 (s, 3H). A yield of the compound 9i was 51%.

Compound 9j:

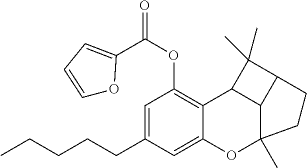 [SMILES: CCCCCc1cc(OC(=O)c2ccco2)c2c(c1)OC1(C)CCC3C1C2C3(C)C]

(raw material including the compound 3a and 2-Furoic acid). NMR data of the compound 9j includes: ¹H NMR (600 MHz, CDCl₃) δ 7.67 (d, J=1.7 Hz, 1H), 7.36 (d, J=3.5 Hz, 1H), 6.64 (s, 1H), 6.59 (dd, J=3.5, 1.7 Hz, 1H), 6.57 (s, 1H), 3.05 (d, J=9.6 Hz, 1H), 2.57-2.49 (m, 3H), 2.35 (t, J=7.5 Hz, 1H), 2.02-1.94 (m, 1H), 1.72-1.67 (m, 1H), 1.64-1.55 (m, 4H), 1.37 (s, 3H), 1.34-1.29 (m, 4H), 1.16 (s, 3H), 0.88 (t, J=6.7 Hz, 3H), 0.80 (s, 3H). A yield of the compound 9j was 37%.

Compound 9k:

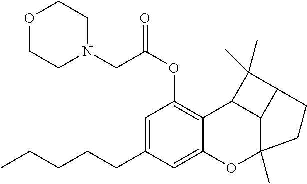 [SMILES: CCCCCc1cc(OC(=O)CN2CCOCC2)c2c(c1)OC1(C)CCC3C1C2C3(C)C]

(raw material including the compound 3a and 4-Morpholineacetic acid). NMR data of the compound 9k includes: ¹H NMR (600 MHz, CDCl₃) δ 6.53 (s, 1H), 6.42 (s, 1H), 3.71 (t, J=4.7 Hz, 4H), 3.43-3.34 (m, 2H), 2.89 (d, J=9.7 Hz, 1H), 2.61 (t, J=4.8 Hz, 4H), 2.48-2.40 (m, 3H), 2.31 (t, J=7.6 Hz, 1H), 1.92-1.84 (m, 1H), 1.65-1.59 (m, 1H), 1.58-1.47 (m, 4H), 1.28 (s, 3H), 1.25 (s, 3H), 1.24-1.20 (m, 4H), 0.80 (t, J=6.7 Hz, 3H), 0.66 (s, 3H). A yield of the compound 9k was 45%.

Compound 9l:

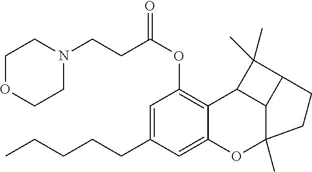 [SMILES: CCCCCc1cc(OC(=O)CCN2CCOCC2)c2c(c1)OC1(C)CCC3C1C2C3(C)C]

(raw material including the compound 3a and 3-(Morpholin-4-yl)propionic acid). NMR data of the compound 91 includes: ¹H NMR (600 MHz, CDCl₃) δ 6.60 (s, 1H), 6.49 (s, 1H), 3.73 (t, J=4.6 Hz, 4H), 2.97 (d, J=9.6 Hz, 1H), 2.80 (t, J=7.2 Hz, 2H), 2.73 (q, J=7.9, 7.2 Hz, 2H), 2.54-2.49 (m, 7H), 2.38 (t, J=7.6 Hz, 1H), 2.01-1.91 (m, 1H), 1.77-1.66 (m, 1H), 1.65-1.54 (m, 4H), 1.35 (s, 3H), 1.31 (s, 3H), 1.32-1.28 (m, 4H), 0.87 (t, J=6.7 Hz, 3H), 0.73 (s, 3H). A yield of the compound 9l was 57%.

Compound 9m:

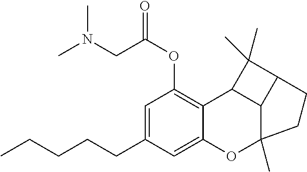 [SMILES: CCCCCc1cc(OC(=O)CN(C)C)c2c(c1)OC1(C)CCC3C1C2C3(C)C]

(raw material including the compound 3a and N, N-Dimethylglycine). NMR data of the compound 9m includes: ¹H NMR (600 MHz, CDCl₃) δ 6.59 (s, 1H), 6.49 (s, 1H), 3.47-3.37 (m, 2H), 2.97 (d, J=9.6 Hz, 1H), 2.57-2.48 (m, 3H), 2.44 (s, 6H), 2.38 (t, J=7.5 Hz, 1H), 1.99-1.91 (m, 1H), 1.72-1.65 (m, 1H), 1.64-1.53 (m, 4H), 1.34 (s, 3H), 1.31 (s, 3H), 1.31-1.27 (m, 4H), 0.87 (t, J=6.7 Hz, 3H), 0.72 (s, 3H). A yield of the compound 9m was 34%.

Compound 9n:

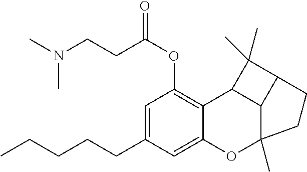 [SMILES: CCCCCc1cc(OC(=O)CCN(C)C)c2c(c1)OC1(C)CCC3C1C2C3(C)C]

(raw material including the compound 3a and 3-(Dimethylamino)propionic acid). NMR data of the compound 9n includes: ¹H NMR (600 MHz, CDCl₃) δ 6.59 (s, 1H), 6.48 (s, 1H), 2.97 (d, J=9.6 Hz, 1H), 2.75-2.69 (m, 4H), 2.57-2.47 (m, 3H), 2.38 (t, J=7.6 Hz, 1H), 2.30 (s, 6H), 2.00-1.90 (m, 1H), 1.73-1.66 (m, 1H), 1.65-1.54 (m, 4H), 1.35 (s, 3H), 1.31 (s, 3H), 1.31-1.28 (m, 4H), 0.87 (t, J=6.7 Hz, 3H), 0.73 (s, 3H). A yield of the compound 9n was 15%.

Compound 9o:

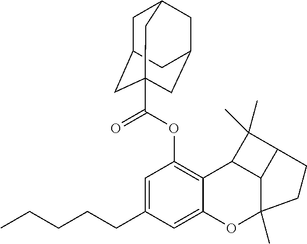 [SMILES: CCCCCc1cc(OC(=O)C23CC4CC(CC(C4)C2)C3)c2c(c1)OC1(C)CCC3C1C2C3(C)C]

(raw material including the compound 3a and 1-1-Adamantaneacetic acid). NMR data of the compound 9o includes: ¹H NMR (600 MHz, CDCl₃) δ 6.61-6.59 (m, 1H), 6.35-6.32 (m, 1H), 3.02 (d, J=9.5 Hz, 1H), 2.51 (dt, J=11.6, 8.5 Hz, 3H), 2.40 (t, J=8.7 Hz, 1H), 2.10-2.05 (m, 8H), 2.01 (dt, J=13.2, 6.6 Hz, 1H), 1.81-1.70 (m, 6H), 1.68-1.60 (m, 2H), 1.59-1.55 (m, 4H), 1.31 (s, 3H), 1.31 (s, 4H), 1.29 (s, 3H), 0.88 (t, J=6.7 Hz, 3H), 0.66 (s, 3H). A yield of the compound 9o was 15%.

Compound 9p:

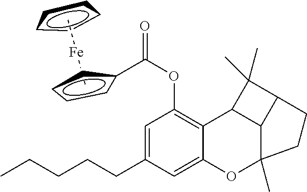

(raw material including the compound 3a and ferrocene carboxylic acid). NMR data of the compound 9p includes: ¹H NMR (600 MHz, CDCl₃) δ 6.63 (s, 1H), 6.56 (s, 1H), 4.94 (s, 2H), 4.50 (s, 2H), 4.32 (s, 5H), 3.03 (d, J=9.6 Hz, 1H), 2.60-2.51 (m, 3H), 2.35 (t, J=7.5 Hz, 1H), 2.02-1.95 (m, 1H), 1.72-1.66 (m, 1H), 1.63-1.56 (m, 4H), 1.36 (s, 3H), 1.35-1.32 (m, 4H), 1.22 (s, 3H), 0.89 (t, J=6.6 Hz, 3H), 0.77 (s, 3H). A yield of the compound 9p was 72%.

Compound 9q:

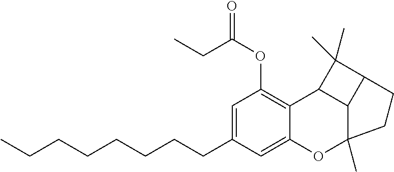 [SMILES: CCCCCCCCc1cc(OC(=O)CC)c2c(c1)OC1(C)CCC3C1C2C3(C)C]

(raw material including the compound 3 h and propionic anhydride, which was synthesized according to Example 5). NMR data of the compound 9q includes: ¹H NMR (600 MHz, CDCl₃) δ 6.59 (s, 1H), 6.48 (s, 1H), 2.97 (d, J=9.6 Hz, 1H), 2.60-2.55 (m, 2H), 2.54-2.49 (m, 3H), 2.38 (t, J=7.5 Hz, 1H), 2.00-1.92 (m, 1H), 1.72-1.67 (m, 1H), 1.64-1.55 (m, 4H), 1.35 (s, 3H), 1.31 (s, 3H), 1.26 (h, J=6.7 Hz, 13H), 0.87 (t, J=6.9 Hz, 3H), 0.73 (s, 3H). A yield of the compound 9q was 47%.

Compound 9r:

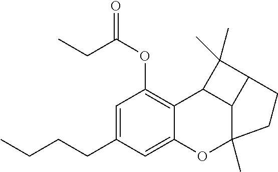 [SMILES: CCCCc1cc(OC(=O)CC)c2c(c1)OC1(C)CCC3C1C2C3(C)C]

(raw material including the compound 3e and propionic anhydride). NMR data of the compound 9r includes: ¹H NMR (600 MHz, CDCl₃) δ 6.59 (s, 1H), 6.48 (s, 1H), 2.97 (d, J=9.6 Hz, 1H), 2.61-2.54 (m, 2H), 2.54-2.50 (m, 3H), 2.38 (t, J=7.5 Hz, 1H), 2.01-1.92 (m, 1H), 1.72-1.67 (m, 1H), 1.64-1.54 (m, 4H), 1.35 (s, 3H), 1.34-1.32 (m, 2H), 1.31 (s, 3H), 1.26 (t, J=7.5 Hz, 3H), 0.90 (t, J=7.4 Hz, 3H), 0.73 (s, 3H). A yield of the compound 9r was 49%.

Compound 9s:

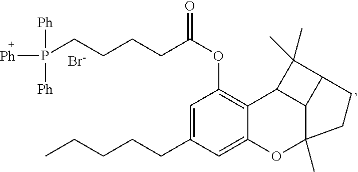

and a method for synthesizing the compound 9s includes: after synthesizing the cannabicyclol derivative 3a according to Example 1, under a nitrogen atmosphere, the compound 3a (157 mg, 0.5 mmol, 1.0 equiv.), 5-Bromopentanoic acid (108.6 mg, 0.6 mmol, 1.2 equiv.), 4-Dimethylaminopyridine (DMAP, 12.2 mg, 20 mol %, 0.02 equiv.), and N,N′-Dicyclohexylcarbodiimide (DCC, 124 mg, 0.6 mmol, 1.2 equiv.) were added to 2 mL of dichloromethane to obtain a reaction mixture. After confirming the completion of a reaction by TLC, the reaction mixture was passed through a short silica gel column, concentrated through rotary evaporation, and purified by silica gel column chromatography to yield a purified compound The purified compound (160 mg, 0.3 mmol, 1.0 equiv.) and triphenylphosphine (157.2 mg, 0.6 mmol, 2.0 equiv.) were added to 5 mL of toluene and stirred at 110° C. for 3 hours to obtain a reaction mixture. After confirming the completion of a reaction by TLC, concentrating the reaction mixture through rotary evaporation, the reaction mixture was purified by silica gel column chromatography, yielding 61 mg of a colorless oily liquid (28% yield), i.e., the compound 9s. NMR data of the compound 9s includes: ¹H NMR (600 MHz, CDCl₃) δ 7.84 (dd, J=12.8, 7.8 Hz, 6H), 7.76 (t, J=7.7 Hz, 3H), 7.70-7.63 (m, 6H), 6.56 (s, 1H), 6.31 (s, 1H), 4.04-3.90 (m, 2H), 2.83 (d, J=9.6 Hz, 1H), 2.71-2.55 (m, 2H), 2.51-2.42 (m, 3H), 2.32 (t, J=7.6 Hz, 1H), 2.17-2.09 (m, 2H), 1.94-1.87 (m, 1H), 1.85-1.78 (m, 2H), 1.68-1.62 (m, 1H), 1.61-1.50 (m, 4H), 1.32 (s, 3H), 1.30-1.26 (m, 4H), 1.19 (s, 3H), 0.88-0.85 (m, 3H), 0.64 (s, 3H).

Compound 9t:

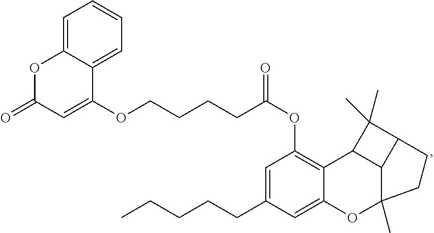 [SMILES: CCCCCc1cc(OC(=O)CCCCOc2cc(=O)oc3ccccc23)c2c(c1)OC1(C)CCC3C1C2C3(C)C]

and a method for synthesizing the compound 9t includes: after synthesizing the cannabicyclol derivative 3a according to Example 1, under a nitrogen atmosphere, the compound 3a (157 mg, 0.5 mmol, 1.0 equiv.), 5-Bromopentanoic acid (108.6 mg, 0.6 mmol, 1.2 equiv.), 4-Dimethylaminopyridine (DMAP, 12.2 mg, 20 mol %, 0.02 equiv.), and N,N′-Dicyclohexylcarbodiimide (DCC, 124 mg, 0.6 mmol, 1.2 equiv.) were added to 2 mL of dichloromethane. After confirming the completion of a reaction by TLC, the reaction mixture was passed through a short silica gel column, concentrated through rotary evaporation, and purified by silica gel column chromatography to yield a purified compound Under a nitrogen atmosphere, the purified compound (95.4 mg, 0.2 mmol, 1.0 equiv.), 4-Hydroxycoumarin (81 mg, 0.5 mmol, 2.5 equiv.), and potassium carbonate (82.8 mg, 0.6, 3.0 equiv.) were added to 10 mL of acetone and stirred at 70° C. to obtain a reaction mixture. After confirming the completion of a reaction by TLC, the reaction mixture was extracted with 25 mL of ethyl acetate three times, and the organic phase was collected. After concentrating the organic phase through rotary evaporation, the organic phase was purified by silica gel column chromatography, yielding 20 mg of a colorless oily liquid (18% yield), i.e., the compound 9t. NMR data of the compound 9t includes: ¹H NMR (600 MHz, CDCl₃) δ 7.83 (d, J=7.8 Hz, 1H), 7.55 (t, J=7.9 Hz, 1H), 7.32 (d, J=8.3 Hz, 1H), 7.26 (t, J=7.2 Hz, 1H), 6.61 (s, 1H), 6.48 (s, 1H), 5.68 (s, 1H), 4.19 (t, J=5.8 Hz, 2H), 2.96 (d, J=9.6 Hz, 1H), 2.73-2.63 (m, 2H), 2.59-2.45 (m, 3H), 2.38 (t, J=7.5 Hz, 1H), 2.08-1.93 (m, 5H), 1.71-1.67 (m, 1H), 1.65-1.54 (m, 4H), 1.35 (s, 3H), 1.30 (s, 3H), 1.30-1.27 (m, 4H), 0.87 (t, J=6.7 Hz, 3H), 0.73 (s, 3H).

### Example 6

### Synthesis of Other Types of Cannabicyclol Derivatives

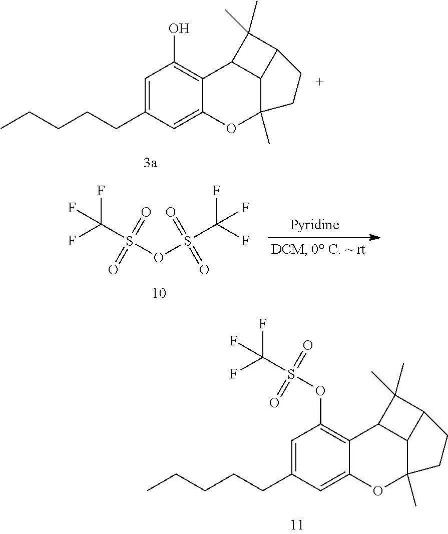 [SMILES: CCCCCc1cc(O)c2c(c1)OC1(C)CCC3C1C2C3(C)C ; O=S(=O)(OS(=O)(=O)C(F)(F)F)C(F)(F)F ; CCCCCc1cc2c(c(OS(=O)(=O)C(F)(F)F)c1)C1C3C(CCC3(C)O2)C1(C)C]

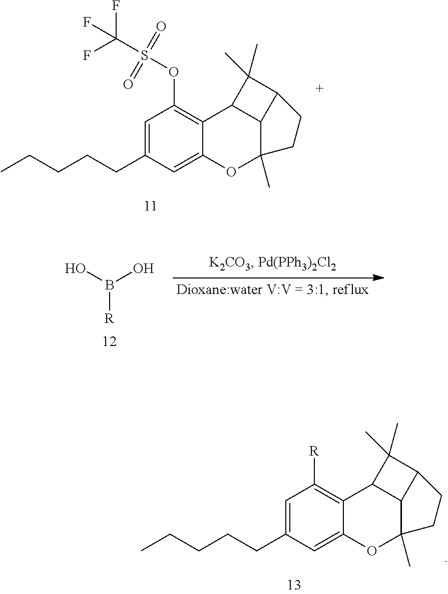

Taking a compound 13a:

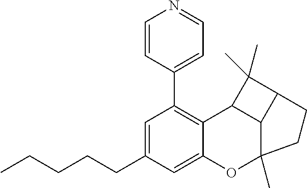 [SMILES: CCCCCc1cc2c(c(-c3ccncc3)c1)C1C3C(CCC3(C)O2)C1(C)C]

as an example, after synthesizing the cannabicyclol derivative 3a according to Example 1, under a nitrogen atmosphere and at 0° C., the compound 3a (314 mg, 1 mmol, 1.0 equiv.), trifluoromethanesulfonic anhydride 10 (423 mg, 1.5 mmol, 1.5 equiv.), and pyridine (173 μL, 1.2 mmol, 1.2 equiv.) were added to 2 mL of dichloromethane to obtain a reaction mixture. After confirming the completion of a reaction by TLC, the reaction mixture was extracted with 25 mL of ethyl acetate three times, and the organic phase was collected. After concentrating the organic phase through rotary evaporation, the organic phase was purified by silica gel column chromatography, yielding 420 mg of a colorless oily liquid (94% yield), i.e., a compound 11.

Under a nitrogen atmosphere, the compound 11 (89.2 mg, 0.2 mmol, 1.0 equiv.), 4-Pyridineboronic acid 12 (110.7 mg, 0.5 mmol, 2.5 equiv.), potassium carbonate (55.2 mg, 0.4 mmol, 2.0 equiv.), and bis(triphenylphosphine)palladium(II) dichloride (7 mg, 5 mol %) were added to a 1,4-dioxane:water mixed solvent (8 mL, V:V=3:1). After confirming the completion of a reaction by TLC, the reaction mixture was extracted with 25 mL of ethyl acetate three times, and the organic phase was collected. After concentrating the organic phase through rotary evaporation, the organic phase was purified by silica gel column chromatography, yielding 67 mg of a colorless oily liquid (89% yield), i.e., the compound 13a. NMR data of the compound 13a includes: ¹H NMR (600 MHz, CDCl₃) δ 8.60 (s, 2H), 7.21 (s, 2H), 6.78 (s, 1H), 6.59 (s, 1H), 3.22 (d, J=9.6 Hz, 1H), 2.56-2.47 (m, 3H), 2.31 (t, J=8.3 Hz, 1H), 2.02-1.94 (m, 1H), 1.76-1.67 (m, 1H), 1.65-1.55 (m, 4H), 1.33-1.30 (m, 4H), 1.29 (s, 3H), 0.87 (t, J=5.8 Hz, 3H), 0.83 (s, 3H), 0.52 (s, 3H).

Using the above synthesis method, a series of C-5 coupled cannabicyclol derivatives were synthesized, including the following cannabicyclol derivatives.

Compound 13b:

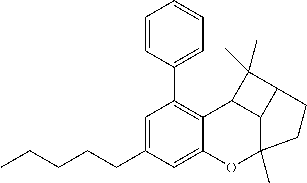 [SMILES: CCCCCc1cc2c(c(-c3ccccc3)c1)C1C3C(CCC3(C)O2)C1(C)C]

(raw material including the compound 11 and benzonic acid). NMR data of the compound 13b includes: ¹H NMR (600 MHz, CDCl₃) δ 7.36 (t, J=7.5 Hz, 2H), 7.30 (t, J=7.4 Hz, 1H), 7.25 (d, J=3.9 Hz, 2H), 6.73 (s, 1H), 6.67 (s, 1H), 3.25 (d, J=9.8 Hz, 1H), 2.60 (t, J=8.5 Hz, 1H), 2.53-2.50 (m, 2H), 2.42 (t, J=7.7 Hz, 1H), 2.04-1.96 (m, 1H), 1.74-1.67 (m, 1H), 1.59-1.55 (m, 4H), 1.40 (s, 3H), 1.32-1.30 (m, 4H), 1.29 (s, 3H), 0.88 (t, J=7.1 Hz, 3H), 0.71 (s, 3H). A yield of the compound 13b was 13%.

Compound 13c:

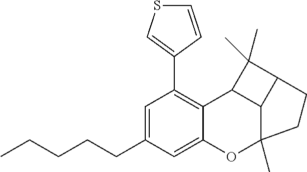 [SMILES: CCCCCc1cc2c(c(-c3ccsc3)c1)C1C3C(CCC3(C)O2)C1(C)C]

(raw material including the compound 11 and 3-Thiopheneboronic acid). NMR data of the compound 13c includes: ¹H NMR (600 MHz, CDCl₃) δ 7.33-7.30 (m, 1H), 7.12 (s, 1H), 7.07 (d, J=4.9 Hz, 1H), 6.72 (s, 1H), 6.69 (s, 1H), 3.36 (d, J=9.7 Hz, 1H), 2.55-2.50 (m, 3H), 2.31 (t, J=6.9 Hz, 1H), 2.03-1.94 (m, 1H), 1.74-1.66 (m, 1H), 1.65-1.56 (m, 4H), 1.31 (s, 6H), 0.90-0.85 (m, 7H), 0.52 (s, 3H). A yield of the compound 13c was 82%.

Compound 13d:

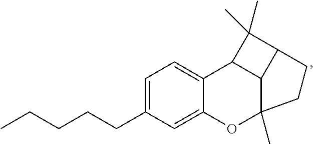 [SMILES: CCCCCc1ccc2c(c1)OC1(C)CCC3C1C2C3(C)C]

and a method for synthesizing the compound 13d includes: under a nitrogen atmosphere, the compound 11 (89.2 mg, 0.2 mmol, 1.0 equiv.), 1,1′-Bis(diphenylphosphino) ferrocene (7.3 mg, 5 mol %, 0.05 equiv.), palladium acetate (11.2 mg, 5 mol %, 0.05 equiv.), triethylamine (300 μL), and formic acid (80 μL) were added to 2 mL of tetrahydrofuran and stirred at 60° C. for 4 hours to obtain a reaction mixture. After confirming the completion of a reaction by TLC, the reaction mixture was extracted with 25 mL of ethyl acetate three times, and the organic phase was collected. After concentrating the organic phase through rotary evaporation, the organic phase was purified by silica gel column chromatography, yielding 67 mg of a colorless oily liquid (89% yield), i.e., the compound 13d. NMR data of the compound 13d includes: ¹H NMR (600 MHz, CDCl₃) δ 6.79 (d, J=7.6 Hz, 1H), 6.70 (s, 1H), 6.68 (s, 1H), 3.02 (d, J=9.6 Hz, 1H), 2.62 (t, J=8.6 Hz, 1H), 2.53 (t, J=7.9 Hz, 2H), 2.39 (t, J=7.6 Hz, 1H), 1.98-1.89 (m, 1H), 1.73-1.66 (m, 1H), 1.65-1.54 (m, 4H), 1.38 (s, 3H), 1.35 (s, 3H), 1.33-1.30 (m, 4H), 0.89 (t, J=6.7 Hz, 3H), 0.72 (s, 3H).

### Examples 7 to 9

### Synthesis of Cannabicyclol by One-Pot Manner with Different Photocatalysts

Preparation methods and raw materials of Examples 7 to 9 are the same as those of Example 1, with the difference being using different catalysts, as shown in Table 1.

TABLE 1
Effect of different photocatalysts on the yield
of synthesized products in a one-pot manner

| Example | Catalyst | Solvent | Yield % |
|---|---|---|---|
| 7 | Ru(bpy)₃Cl₂•6H₂O | Toluene | <5 |
| 8 | [Ir[dFCF₃ppy]₂(dtbpy)]PF₆ | Toluene | 23 |
| 9 | [Ir[dFCF₃ppy]₂(bpy)]PF₆ | Toluene | 39 |

### Example 10

### Synthesis of a Cannabicyclol Derivative

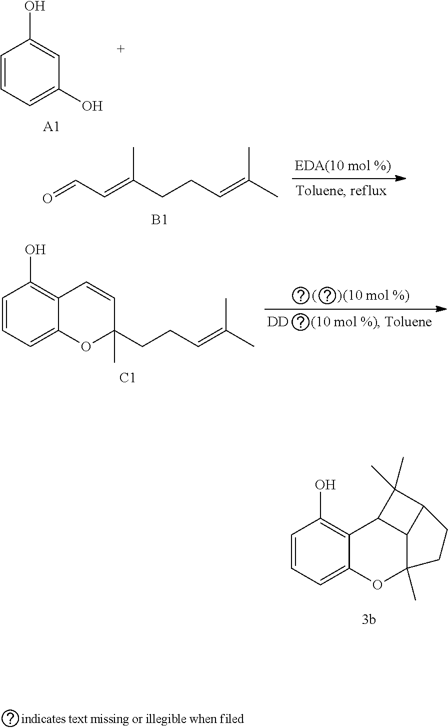

The cannabicyclol derivative is prepared by the following steps.

Step 1) Resorcinol A1 (0.2 mmol, 1.0 equiv.), citral B1 (0.3 mmol, 1.5 equiv.), toluene (2.0 mL), and ethylenediamine (EDA) (0.02 mmol, 0.1 equiv.) were added sequentially to a reaction tube under a nitrogen atmosphere. The reaction was carried out under reflux in toluene at 110° C., and the reaction process was monitored by TLC, after confirming the completion of the reaction by TLC, the reaction was quenched with an aqueous solution of ammonium chloride to obtain a reaction mixture. The reaction mixture was extracted with ethyl acetate, and the organic layers were dried over anhydrous sodium sulfate. After removal of the solvent of the organic layers under reduced pressure, the residue was purified by silica gel column chromatography to yield 24.7 mg of a yellow oily liquid (55% yield), i.e., a compound C1.

Step 2) Under a nitrogen atmosphere, indium trifluoromethanesulfonate (0.01 mmol, 0.1 equiv.), 2,3-dichloro-5,6-dicyano-1,4-benzoquinone (DDQ, 0.01 mmol, 0.1 equiv.), toluene (2.0 mL) and the compound C1 (0.1 mmol, 1.0 equiv.) were added sequentially to a reaction tube. The reaction was carried out at room temperature, and the reaction process was monitored by TLC, after confirming the completion of the reaction by TLC, the reaction was quenched with an aqueous solution of ammonium chloride to obtain a reaction mixture. The reaction mixture was extracted with ethyl acetate, and the organic layers were dried over anhydrous sodium sulfate. After removal of a solvent from the organic layers under reduced pressure, the residue was purified by silica gel column chromatography to yield 18.3 mg of white solid (75% yield), i.e., a compound 3b. NMR of the compound 3b includes: ¹H NMR (500 MHz, CD₃OD) δ 6.86 (t, J=8.0 Hz, 1H), 6.32 (dd, J=8.0, 1.1 Hz, 1H), 6.29 (dd, J=8.1, 1.1 Hz, 1H), 3.13 (d, J=9.6 Hz, 1H), 2.53 (t, J=8.6 Hz, 1H), 2.37 (t, J=7.2 Hz, 1H), 2.02-1.90 (m, 1H), 1.72-1.51 (m, 3H), 1.36 (s, 3H), 1.33 (s, 3H), 0.76 (s, 3H).

### Example 11

### Synthesis of a Cannabicyclol Derivative

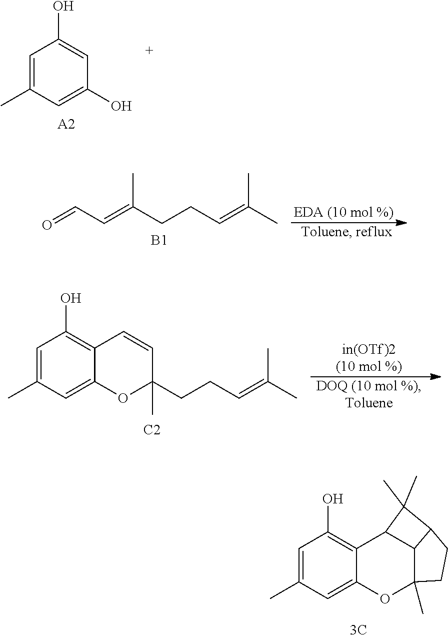

The cannabicyclol derivative is synthesized by the following steps.

Step 1) Under a nitrogen atmosphere, 3-Methylresorcinol A2 (3 mmol, 1.5 equiv.), citral B1 (2 mmol, 1.0 equiv.), toluene (5.0 mL), and ethylenediamine (EDA) (0.02 mmol, 0.1 equiv.) were added sequentially to a reaction tube. The reaction was carried out under reflux in toluene at 110° C., and the reaction process was monitored by TLC, after confirming the completion of the reaction by TLC, the reaction was quenched with an aqueous solution of ammonium chloride to obtain a reaction mixture. The reaction mixture was extracted with ethyl acetate, and the organic layers were dried over anhydrous sodium sulfate. After removal of a solvent from the organic layers under reduced pressure, the residue was purified by silica gel column chromatography to yield 280 mg of a yellow oily liquid (in 54% yield), i.e., the compound C1.

Step 2) Under a nitrogen atmosphere, indium trifluoromethanesulfonate (0.02 mmol, 0.1 equiv.), 2,3-dichloro-5,6-dicyano, 4-benzoquinone (DDQ, 0.02 mmol, 0.1 equiv.), toluene (2.0 mL), and C2 (0.2 mmol, 1.0 equiv.) were sequentially added to a reaction tube. The reaction was carried out under reflux in toluene at 110° C., and the reaction process was monitored by TLC, after confirming the completion of the reaction by TLC, the reaction was quenched with an aqueous solution of ammonium chloride to obtain a reaction mixture. The reaction mixture was extracted with ethyl acetate, and the organic layers were dried over anhydrous sodium sulfate. After removal of a solvent of the organic layers under reduced pressure, the residue was purified by silica gel column chromatography to yield 54.1 mg of white solid (87% yield), i.e., a compound 3c. NMR data of the compound 3c includes: ¹H NMR (600 MHz, CDCl₃) δ 6.32 (s, 1H), 6.17 (s, 1H), 4.61 (s, 1H), 3.07 (d, J=9.6 Hz, 1H), 2.63-2.52 (m, 1H), 2.45-2.35 (m, 1H), 2.22 (s, 3H), 2.03-1.95 (m, 1H), 1.64-1.46 (m, 3H), 1.38 (s, 6H), 0.79 (s, 3H).

### Example 12

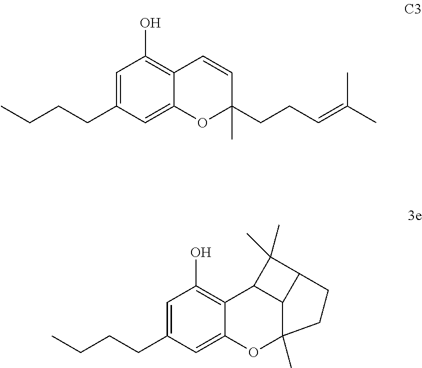

A cannabicyclol derivative is synthesized by the following steps.

3-Butylresorcinol (1.8 mmol, 1.0 equiv.), citral B1 (2.7 mmol, 1.5 equiv.), toluene (5.0 mL), and ethylenediamine (EDA) (0.2 mmol, 0.1 equiv.) were used as raw material and synthesized according to Example 11 to yield 166 mg of a yellow oily liquid (55% yield), i.e., a compound C3.

The compound C3 (1 mmol, 1.0 equiv.), indium trifluoromethanesulfonate (0.1 mmol, 0.1 equiv.), 4-Benzoquinone (DDQ) (0.1 mmol, 0.1 equiv.), and toluene (5.0 mL) were used as raw materials and synthesized according to Example 11 to yield 80.1 mg of white solid (48% yield), i.e., a compound 3e. NMR data of the compound 3e includes: ¹HNMR (600 MHz, CDCl₃) δ 6.33 (s, 1H), 6.18 (s, 1H), 4.56 (s, 1H), 3.06 (d, J=9.6 Hz, 1H), 2.64-2.51 (m, 1H), 2.51-2.43 (m, 2H), 2.39 (t, J=7.4 Hz, 1H), 2.04-1.92 (m, 1H), 1.71-1.52 (m, 5H), 1.38 (s, 3H), 1.38 (s, 3H), 1.35-1.30 (m, 2H), 0.90 (t, J=7.4 Hz, 3H), 0.80 (s, 3H).

### Example 13

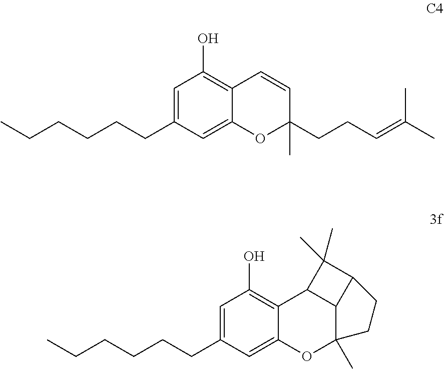

A cannabicyclol derivative is synthesized by the following steps.

Step 1) 3-Hexylresorcinol (1.9 mmol, 1.0 equiv.), citral B1 (2.9 mmol, 1.5 equiv.), toluene (5.0 mL), and ethylenediamine (EDA) (0.2 mmol, 0.1 equiv.) were used as raw material and synthesized according to Example 11 to yield 328.2 mg of a yellow oily liquid (55% yield), i.e., a compound C4.

Step 2) The compound C4 (1 mmol, 1.0 equiv.), indium trifluoromethanesulfonate (0.1 mmol, 0.1 equiv.), 4-Benzoquinone (DDQ) (0.1 mmol, 0.1 equiv.), and toluene (5.0 mL) were used as raw materials and synthesized according to Example 11 to yield 80.3 mg of a white solid (22% yield), i.e., a compound 3f. NMR data of the compound 3f includes: ¹H NMR (600 MHz, CDCl₃) δ 6.33 (s, 1H), 6.17 (s, 1H), 4.66 (s, 1H), 3.07 (d, J=9.6 Hz, 1H), 2.63-2.54 (m, 1H), 2.47-2.43 (m, 2H), 2.40 (t, J=7.4 Hz, 1H), 1.99 (m, 1H), 1.74-1.52 (m, 5H), 1.39 (s, 3H), 1.38 (s, 3H), 1.32-1.25 (m, 6H), 0.87 (t, J=6.6 Hz, 3H), 0.80 (s, 3H).

### Example 14

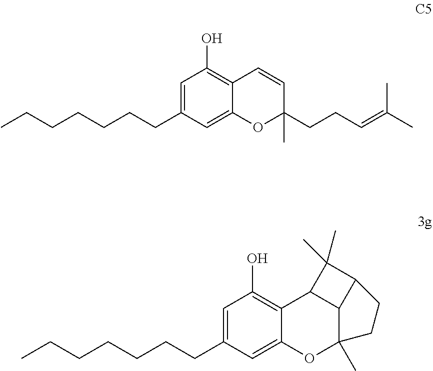

A cannabicyclol derivative is synthesized by the following steps.

Step 1) 3-Heptylresorcinol (1.5 mmol, 1.0 equiv.), citral B1 (2.3 mmol, 1.5 equiv.), toluene (5.0 mL), and ethylenediamine (EDA) (0.15 mmol, 0.1 equiv.) were used as raw material and synthesized according to Example 11 to yield 245 mg of a yellow oily liquid (47% yield), i.e., a compound C5.

Step 2) The compound C5 (0.6 mmol, 1.0 equiv.), indium trifluoromethanesulfonate (0.06 mmol, 0.1 equiv.), 4-Benzoquinone (DDQ) (0.06 mmol, 0.1 equiv.), and toluene (3.0 mL) were used as raw materials and synthesized according to Example 11 to yield 150.1 mg of white solid (70% yield), i.e., a compound 3g. NMR data of the compound 3g includes: ¹H NMR (600 MHz, CDCl₃) δ 6.32 (s, 1H), 6.17 (s, 1H), 4.56 (s, 1H), 3.06 (d, J=9.6 Hz, 1H), 2.60-2.54 (m, 1H), 2.48-2.42 (m, 2H), 2.39 (t, J=7.4 Hz, 1H), 2.03-1.95 (m, 1H), 1.71-1.52 (m, 5H), 1.38 (s, 3H), 1.38 (s, 3H), 1.30-1.25 (m, 8H), 0.87 (t, J=6.8 Hz, 3H), 0.80 (s, 3H).

### Example 15

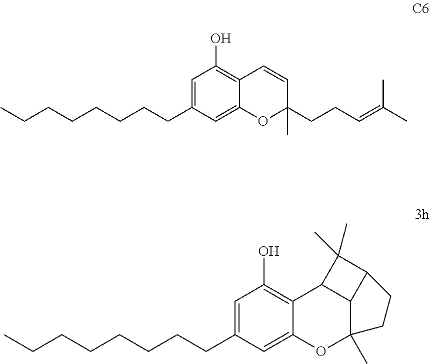

A cannabicyclol derivative is synthesized by the following steps.

Step 1) 3-Octylresorcinol (1.3 mmol, 1.0 equiv.), citral B1 (2.0 mmol, 1.5 equiv.), toluene (5.0 mL), and ethylenediamine (EDA) (0.13 mmol, 0.1 equiv.) were used as raw material and synthesized according to Example 11 to yield 215 mg of a colorless oily liquid (yield 46%), i.e, a compound C6.

Step 2) The compound C6 (0.7 mmol, 1.0 equiv.), indium trifluoromethanesulfonate (0.07 mmol, 0.1 equiv.), 4-Benzoquinone (DDQ) (0.07 mmol, 0.1 equiv.), and toluene (2.0 mL) were used as raw materials and synthesized according to Example 11 to yield 176.4 mg of white solid (72% yield), i.e., a compound 3h. NMR data of the compound 3h includes: ¹H NMR (600 MHz, CDCl₃) δ 6.32 (s, 1H), 6.18 (s, 1H), 4.50 (s, 1H), 3.06 (d, J=9.6 Hz, 1H), 2.60-2.53 (m, 1H), 2.48-2.42 (m, 2H), 2.39 (t, J=7.4 Hz, 1H), 2.02-1.94 (m, 1H), 1.71-1.52 (m, 5H), 1.38 (s, 3H), 1.37 (s, 3H), 1.31-1.23 (m, 10H), 0.87 (t, J=6.9 Hz, 3H), 0.80 (s, 3H).

### Example 16

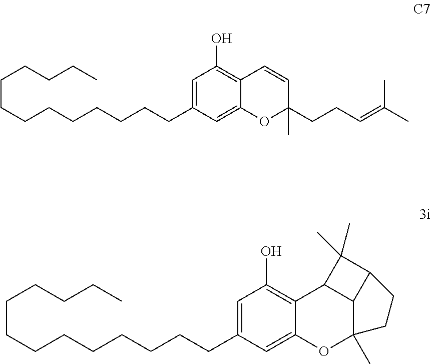 [SMILES: CCCCCCCCCCCCCc1cc(O)c2c(c1)OC(C)(CCC=C(C)C)C=C2 ; CCCCCCCCCCCCCc1cc(O)c2c(c1)OC1(C)CCC3C1C2C3(C)C]

A cannabicyclol derivative is synthesized by the following steps.

Step 1) 3-Tridecylresorcinol (0.98 mmol, 1.0 equiv.), citral B1 (1.5 mmol, 1.5 equiv.), toluene (2.0 mL), and ethylenediamine (EDA) (0.1 mmol, 0.1 equiv.) were used as raw material and synthesized according to Example 11 to yield 406.3 mg of a colorless oily liquid (92% yield), i.e., a compound C7.

Step 2) The compound C7 (0.4 mmol, 1.0 equiv.), indium trifluoromethanesulfonate (0.04 mmol, 0.1 equiv.), 4-Benzoquinone (DDQ) (0.04 mmol, 0.1 equiv.), and toluene (2.0 mL) were used as raw materials and synthesized according to Example 11 to yield 102 mg of white solid (60% yield), i.e., a compound 3i. NMR data of the compound 3i includes: ¹H NMR (600 MHz, CDCl₃) δ 6.32 (s, 1H), 6.17 (s, 1H), 4.54 (s, 1H), 3.06 (d, J=9.6 Hz, 1H), 2.61-2.53 (m, 1H), 2.49-2.42 (m, 2H), 2.39 (t, J=7.4 Hz, 1H), 2.02-1.95 (m, 1H), 1.70-1.52 (m, 5H), 1.38 (s, 3H), 1.38 (s, 3H), 1.30-1.23 (m, 20H), 0.88 (t, J=6.9 Hz, 3H), 0.80 (s, 3H).

### Example 17

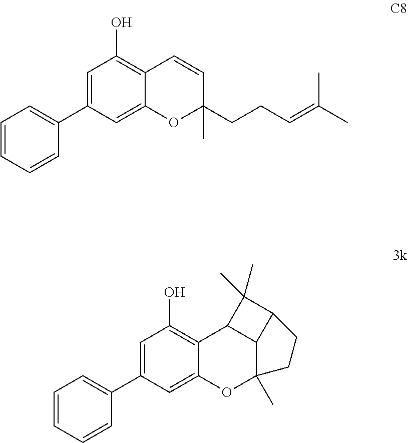 [SMILES: CC(C)=CCCC1(C)C=Cc2c(O)cc(-c3ccccc3)cc2O1 ; CC12CCC3C1C(c1c(O)cc(-c4ccccc4)cc1O2)C3(C)C]

A cannabicyclol derivative is synthesized by the following steps.

Step 1) 3-Phenylresorcinol (0.35 mmol, 1.0 equiv.), citral B1 (0.6 mmol, 1.5 equiv.), toluene (2.0 mL), and ethylenediamine (EDA) (0.04 mmol, 0.1 equiv.) were used as raw material and synthesized according to Example 11 to yield 65.5 mg of a yellow oily liquid (57% yield), i.e., a compound C8.

Step 2) The compound C8 (0.1 mmol, 1.0 equiv.), indium trifluoromethanesulfonate (0.01 mmol, 0.1 equiv.), 4-Benzoquinone (DDQ) (0.01 mmol, 0.1 equiv.), and toluene (2.0 mL) were used as raw materials and synthesized according to Example 11 to yield 32 mg of a white solid (90% yield), i.e., a compound 3k. NMR data of the compound 3k includes: ¹H NMR (500 MHz, CD₃OD) δ 7.54 (d, J=1.4 Hz, 1H), 7.52 (s, 1H), 7.39-7.33 (m, 2H), 7.29-7.22 (m, 1H), 6.61 (d, 1H), 6.58 (d, J=2.3 Hz, 1H), 3.16 (d, J=9.6 Hz, 1H), 2.62-2.51 (m, 1H), 2.45-2.33 (m, 1H), 2.08-1.96 (m, 1H), 1.74-1.55 (m, 3H), 1.38 (d, J=2.5 Hz, 3H), 1.36 (d, J=5.2 Hz, 3H), 0.81 (s, 3H).

### Example 18

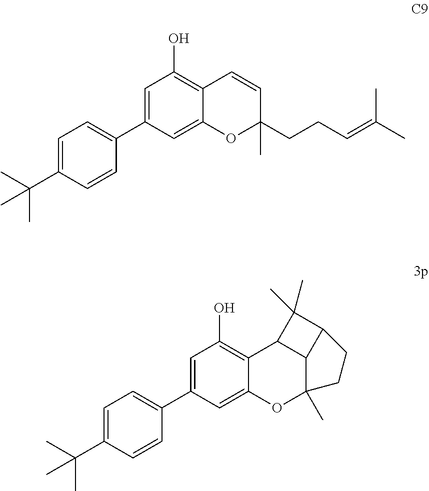

A cannabicyclol derivative is synthesized by the following steps.

Step 1) 3-(4-Tert-butylphenyl)-resorcinol (2 mmol, 1.0 equiv.), citral B1 (3 mmol, 1.5 equiv.), toluene (8.0 mL), and ethylenediamine (EDA) (0.2 mmol, 0.1 equiv.) were used as raw material and synthesized according to Example 11 to yield 400 mg of a yellow oily liquid (53% yield), i.e., a compound C9.

Step 2) The compound C9 (0.2 mmol, 1.0 equiv.), indium trifluoromethanesulfonate (0.02 mmol, 0.1 equiv.), 4-Benzoquinone (DDQ) (0.02 mmol, 0.1 equiv.), and toluene (2.0 mL) were used as raw materials and synthesized according to Example 11 to yield 29 mg of a white solid (43% yield), i.e., a compound C9. NMR data of the compound C9 includes: ¹H NMR (500 MHz, CDCl₃) δ 7.48-7.47 (m, 1H), 7.46 (d, J=1.9 Hz, 1H), 7.42-7.37 (m, 2H), 6.62 (s, 1H), 6.58 (s, 1H), 3.16 (d, J=9.6 Hz, 1H), 2.59-2.51 (m, 1H), 2.42-2.34 (m, 1H), 2.08-1.93 (m, 1H), 1.74-1.53 (m, 3H), 1.38 (d, J=2.3 Hz, 3H), 1.35 (d, J=2.0 Hz, 3H), 1.34-1.31 (m, 10H), 0.81 (s, 3H).

### Example 19

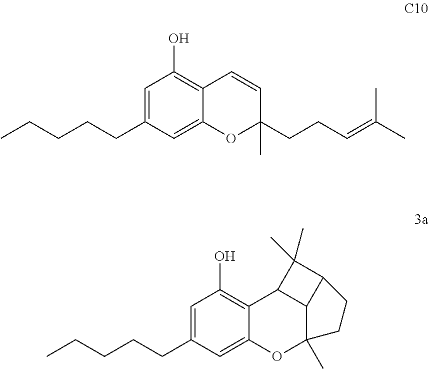

A cannabicyclol derivative is synthesized by the following steps.

Step 1) Olive alcohol (2 mmol, 1.0 equiv.) and citral B1 (2 mmol, 1.5 equiv.) were used as raw materials and synthesized according to Example 11 to yield 320.5 mg of a light yellow oily liquid (51% yield), i.e., a compound C10.

Step 2) The compound C10 (0.2 mmol, 1.0 equiv.), indium trifluoromethanesulfonate (0.02 mmol, 0.1 equiv.), 4-Benzoquinone (DDQ) (0.02 mmol, 0.1 equiv.), and toluene (2.0 mL) were used as raw materials and synthesized according to Example 11 to yield 49.4 mg of a white solid (79% yield), i.e., the compound 3a. NMR data of the compound 3a includes: ¹H NMR (600 MHz, CDCl₃) δ 6.32 (s, 1H), 6.18 (d, J=1.5 Hz, 1H), 4.43 (s, 1H), 3.05 (d, J=9.6 Hz, 1H), 2.64-2.53 (m, 1H), 2.52-2.42 (m, 2H), 2.39 (t, J=7.4 Hz, 1H), 2.02-1.91 (m, 1H), 1.74-1.52 (m, 5H), 1.38 (s, 3H), 1.37 (s, 3H), 1.30 (tq, J=9.1, 5.8, 5.2 Hz, 4H), 0.87 (t, J=6.8 Hz, 3H), 0.79 (s, 3H).

### Examples 20 to 29

Preparation methods and raw materials in Examples 20 to 29 are the same as those of Example 19, with the difference being using different catalysts, as shown in Table 2.

TABLE 2
Effect of different catalysts on yield

| Example | Catalyst | Solvent | Oxidizing agent | Yield % |
|---|---|---|---|---|
| 20 | FeCl₃ (10%) | Toluene | DDQ (10%) | 60 |
| 21 | CuBr (10%) | Toluene | DDQ (10%) | <5 |
| 22 | ZnCl₂ (10%) | Toluene | DDQ (10%) | <5 |
| 23 | In(OTf)₃ (10%) | Toluene | DDQ (10%) | 79 |
| 24 | BF₃•Et₂O (10%) | Toluene | DDQ (10%) | 73 |
| 25 | Cu(OTf)₂ | Toluene | DDQ (10%) | 70 |
| 26 | InBr₃ | Toluene | DDQ (10%) | 71 |
| 27 | Sc(OTf)₂ | Toluene | DDQ (10%) | 71 |
| 28 | AgOTf | Toluene | DDQ (10%) | 10 |
| 29 | InCl₃ | Toluene | DDQ (10%) | 19 |

### Examples 30 to 35

Preparation methods and raw materials in Examples 30 to 35 are the same as those of Example 19, with the difference being using different solvents, as shown in Table 3.

TABLE 3
Effects of different solvents on yield

| | | | Oxidizing | |
|---|---|---|---|---|
| Example | Catalyst | Solvent | agent | Yield % |
| 30 | In(OTf)₃ (10%) | Dichloromethane | DDQ (10%) | 47 |
| 31 | In(OTf)₃ (10%) | Dichloroethane | DDQ (10%) | 72 |
| 32 | In(OTf)₃ (10%) | Acetonitrile | DDQ (10%) | 33 |
| 33 | In(OTf)₃ (10%) | Tetrahydrofuran | DDQ (10%) | <5 |
| 34 | BF₃•Et₂O (10%) | Chlorobenzene | DDQ (10%) | 65 |
| 35 | In(OTf)₃ (10%) | Fluorobenzene | DDQ (10%) | 78 |

### Examples 36 to 37

Preparation methods and raw materials in Examples 36 to 37 are the same as those of Example 19, with the difference being using catalysts and oxidizing agents at different concentrations, as shown in Table 4.

TABLE 4
Effect of catalysts and oxidizing agents
at different concentrations on yield

| Example | Catalyst | Solvent | Oxidizing agent | Yield % |
|---|---|---|---|---|
| 36 | In(OTf)₃ (10%) | Toluene | DDQ (0%) | 49 |
| 37 | In(OTf)₃ (0%) | Toluene | DDQ (10%) | 0 |

As can be seen from Examples 10 to 37, the present disclosure provides cannabicyclol derivatives and preparation methods thereof. The present disclosure provides new methods for synthesizing cannabicyclol and cannabicyclol derivatives with a simple reaction, fast efficiency, high yield, and 100% atom utilization. Compared to previously reported methods in the art, the method disclosed in the present disclosure can utilize catalytic amounts of metal catalysts to facilitate the complete reaction and significantly improve the yield.

### Application Example 1

### Viability Screening of Cannabicyclol and Cannabicyclol Derivatives

The purpose of the application example 1 is to explore the killing effect of cannabicyclol and the cannabicyclol derivatives on a variety of cancer stem cells (CSCs).

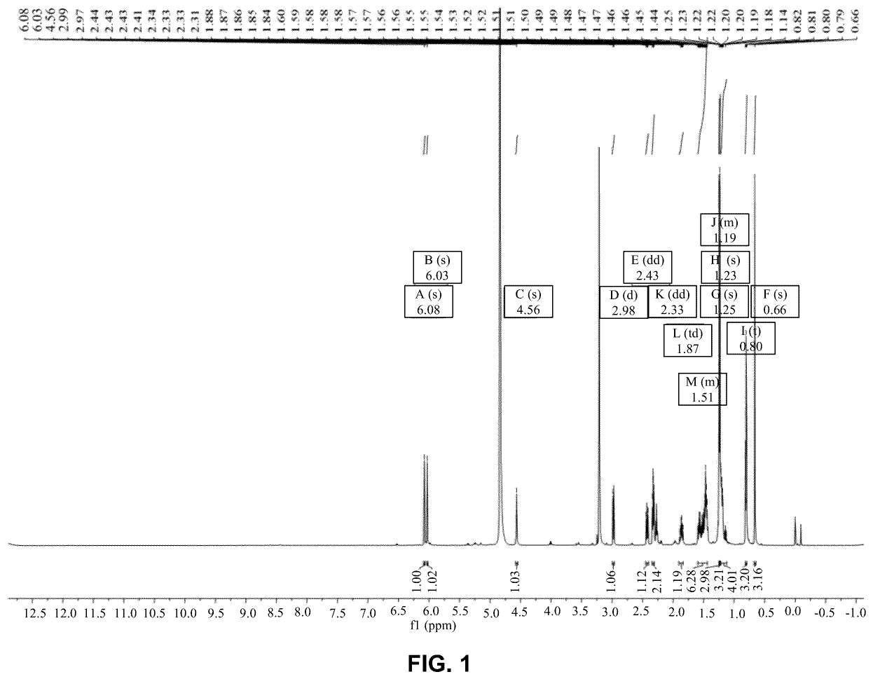

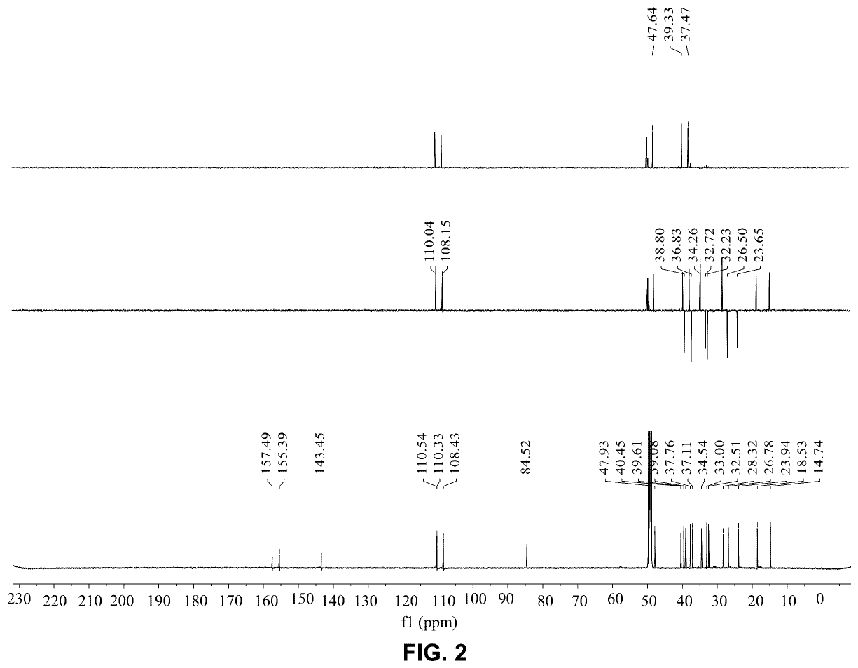

The compound 3a prepared in in Example 1 was analyzed through ¹H-NMR (600 MHz, CD₃OD) and ¹³C-NMR (151 MHz, CD₃OD). A ¹H-NMR pattern of the compound 3a is shown in FIG. 1, and a ¹³C-NMR pattern of the compound 3a is shown in FIG. 2. From FIG. 1 and FIG. 2, it is clear that the cannabicyclol used in the application example 1 is structurally correct, and the purity of the cannabicyclol (CBL) is >98%, which allows for further experiments.

Test samples: cannabicyclol and cannabicyclol derivatives 3a to 13d prepared in Examples 1 to 6 were structurally correct after being analyzed through ¹H-NMR (600 MHz, CDCl₃) and ³C-NMR (151 MHz, CDCl₃), which allows for further experiments.

Primary viability screening: \textcircled{1} the synthesized compounds 3a to 13d were respectively dissolved in DMSO to prepare a 40 μg/mL cannabicyclol and cannabicyclol derivative solutions.

\textcircled{2} A control group: a 200 μL DMSO solvent.

\textcircled{3} The solutions of compounds 3a to 13d were diluted in a twofold gradient to obtain cannabicyclol and cannabicyclol derivative solutions at concentrations of 40 μg/mL, 20 μg/mL, 10 μg/mL, 5 μg/mL, 2.5 μg/mL, 1.25 μg/mL, and 0.625 μg/mL, and 0.3125 μg/mL, respectively.

\textcircled{4} Three glioblastoma stem cells (GSC-3, GSC-12, and GSC-18), three pancreatic cancer cell-derived stem cells (PANC-1-CSC, BXPC-3-CSC, and ASPC1-CSC), three liver cancer cell-derived stem cells (HepG2-CSC, MHCC97H-CSC, and SMMC7721-CSC), HEK-293T cells, 16 tumor cells (U251, U87, T98G, PANC1, BXPC3, ASPC1, HepG2, MHCC97H, SMMC7721, PC3, SKOV3, MDA-MB231, A549, HCT116, HEC-1B, MKN28), and differentiated glioblastoma stem cells (GSC-3diff and GSC-12diff) derived from GSC-3 and GSC-12 (all of the cells were provided by the Laboratory of Tumor Animal Model Preparation and Application Research, West China Hospital, Sichuan University) were inoculated into a 96-well plate at a concentration of 2×10⁴ cells/well, and 100 μL of culture medium was then added to each well;

\textcircled{5} 2) HEK-293T, GSC-3diff, GSC-12diff, and the 16 tumor cells were added with DMEM, 10% FBS, and 1% double antibody culture medium. Cancer stem cells (CSCs) were added with serum-free stem cell medium consisting of DMEM/F12, EGF (20 ng/mL), bFGF (20 ng/mL), B27 (1×), and 1% double antibody culture medium.

\textcircled{6} Inoculated cells were put into a cell culture incubator for 24 hours, and after the cells were well adhered to the wall, the culture medium in the 96-well plate was discarded.

\textcircled{7} 200 μL cannabicyclol and cannabicyclol derivatives solutions at different concentrations were added to the cultured cells.

\textcircled{8} The experiment was set up with 3 replicates.

\textcircled{9} The 96-well plate was placed in a cell culture incubator, and incubation was continued at 37° C. and 5% CO₂ for 72 hours. Then, 100 μL of cannabicyclol and cannabicyclol derivative solution per well was discarded (100 μL of DMSO solvent was discarded for the control group), and 20 μL of LMTS (CellTiter AQueous Non-Radioactive Cell Proliferation Assay, #G3581) was mixed and continued to be placed in the cell incubator for 2 hours of incubation.

\textcircled{10} The absorbance value at 490 nm was measured on an enzyme labeling apparatus, and the IC₅₀ value was calculated.

Applying the above method, cannabicyclol and the cannabicyclol derivatives were subjected to primary viability screening, and data from the primary viability screening were summarized and shown in Table 5.

TABLE 5
Primary viability screening of some cannabicyclol
derivatives against 3#-GSC, U251, and 293T

| | Cell | Cell | Cell |
|---|---|---|---|
| | viability | viability | viability |
| | in 3#-GSC | in U251 | in 293T |
| Compound | (%) | (%) | (%) |
| 3a | 20 | 54 | 82 |
| 3b | 32 | 97 | 97 |
| 3c | ≥100 | 88 | ≥100 |
| 3d | 40 | 71 | 91 |
| 3e | 30 | 17 | 60 |
| 3f | 20 | 78 | 18 |
| 3g | 90 | 79 | 11 |
| 3h | 87 | 75 | 16 |
| 3i | 33 | 79 | 8 |
| 3k | 52 | 15 | 45 |
| 3l | 51 | 45 | 62 |
| 3m | 42 | 90 | ≥100 |
| 3n | 41 | 17 | 50 |
| 3o | 64 | 88 | 81 |
| 3p | 67 | 97 | ≥100 |
| 3q | 39 | 19 | 40 |
| 3r | 43 | 76 | 45 |
| 3s | 49 | 91 | 64 |
| 3t | 50 | 98 | ≥100 |
| 3u | 30 | 72 | 78 |
| 3v | 30 | 71 | 79 |
| 3x | 70 | 77 | 97 |
| 3y | 47 | 98 | ≥100 |
| 7a | ≥100 | 81 | 77 |
| 7b | ≥100 | 91 | 95 |
| 7c | ≥100 | 80 | 79 |
| 7d | ≥100 | 82 | 77 |
| 7e | ≥100 | 72 | 75 |
| 7h | 72 | ≥100 | 87 |
| 7i | 50 | 21 | 71 |
| 9a | 52 | ≥100 | ≥100 |
| 9b | 28 | 82 | 92 |
| 9c | 28 | ≥100 | ≥100 |
| 9e | ≥100 | ≥100 | ≥100 |
| 9f | 41 | 64 | 35 |
| 9g | ≥100 | ≥100 | ≥100 |
| 9h | 90 | ≥100 | ≥100 |
| 9i | 91 | 88 | 91 |
| 9j | ≥100 | ≥100 | ≥100 |
| 9k | 43 | ≥100 | ≥100 |
| 9l | 32 | 90 | 93 |
| 9m | 42 | ≥100 | ≥100 |
| 9n | 32 | 89 | ≥100 |
| 9p | 80 | 98 | 98 |
| 9s | 40 | 17 | 21 |
| 9t | ≥100 | ≥100 | ≥100 |
| 13b | 50 | ≥100 | 75 |

Based on the preliminary viability screening results of cannabicyclol and cannabicyclol derivatives against 3 #-GSC, 293T, and U251, among the cannabicyclol derivatives 3a to 3i with varying alkyl chain lengths, the compound with no alkyl substitution (the compound 3b) exhibited selective anti-glioma stem cell viability, and cannabicyclol derivative substituted with pentyl (the compound 3a) or hexyl (the compound 3f) showed a certain degree of selective anti-glioma stem cell viability. Among the cannabicyclol derivatives 3k to 3t substituted with aryl, substituted aryl, or heteroaryl, except for the meta-methylphenyl (the compound 3m), para-tert-butylphenyl (compound 3p), and heteroaryl (3t), which exhibited weak selective anti-glioma stem cell viability, no significant selective anti-glioma stem cell viability was observed for the aryl-substituted products. The viability data of cannabicyclol derivatives with different substituents introduced at the C-12 position showed that compounds 3u to 3z exhibited certain selective anti-glioma stem cell viability. The preliminary viability screening of cannabicyclol derivatives at the 5-OH position showed that the anti-glioma stem cell viability of the etherification products 7a to 7i was lost, while when the 5-OH of cannabicyclol was coupled with phenyl (the compound 13b), both the viability and selectivity were significantly reduced. The anti-glioma stem cell viability of the esterification compounds 9a to 9t indicated that when acetyl (the compound 9b) or propionyl (the compound 9c) was introduced, both the viability and selectivity were maintained. When a certain water-soluble alkylamine (compounds 9k to 9n) was introduced, the anti-glioma stem cell viability and selectivity were not only maintained but also enhanced.

### Application Example 2

Using the scheme in the application Example 1, the IC₅₀ values of the cannabicyclol derivative (the compound 3a) against 9 types of cancer stem cells are shown in Table 6. The IC₅₀ values of the cannabicyclol derivative (the compound 3a) against other tumor cells (HEK-293T, GSC-3diff, GSC-12diff, and 16 cancer stem cells) are shown in Table 7.

TABLE 6
IC₅₀ values of CBL against different cancer stem cells

| | Tumor stem cell | | | | |
|---|---|---|---|---|---|
| | | | | PANC1- | BXPC3- |
| | GSC-3 | GSC-12 | GSC-18 | CSC | CSC |
| IC₅₀ (μg/mL) | 3.85 | 8.03 | 6.40 | 12.99 | 2.19 |
| | Tumor stem cell | | | | |
| | ASPC1- | HepG2- | MHCC97H- | SMMC7721- | |
| | CSC | CSC | CSC | CSC | — |
| IC₅₀ (μg/mL) | 14.57 | 2.37 | 5.32 | 3.85 | — |

As shown in Table 6, the CBL exhibited significant inhibitory activity against 9 types of cancer stem cells. As shown in Table 6, the IC₅₀ values of the CBL against these 9 types of cancer stem cells are in a range of 37 to 14.57 μg/mL. The CBL demonstrated the strongest cytotoxicity against BXPC-3-CSC and HepG2-CSC, while exhibiting relatively weaker cytotoxicity against PANC-1-CSC and ASPC1-CSC.

TABLE 7
IC₅₀ values of CBL against HEK-293T, GSC-3diff, GSC-12diff and 16 tumor cells

| Tumor cell | HEK-293T | U251 | U87 | T98G | PANC1 | BXPC3 | SKOV3 |
|---|---|---|---|---|---|---|---|
| IC₅₀(μg/mL) | >40 | >40 | >40 | >40 | >40 | >40 | >40 |

| Tumor cell | MDA-MB231 | A549 | HCT116 | HEC18 | MKN28 | GSC-3diff | GSC-12diff |
|---|---|---|---|---|---|---|---|
| IC₅₀(μg/mL) | >40 | >40 | >40 | >40 | >40 | 23.48 | 29.64 |

| Tumor cell | ASPC1 | HepG2 | MHCC97 | SMMC7721 | PC3 | — |
|---|---|---|---|---|---|---|
| IC₅₀(μg/mL) | >40 | >40 | >40 | >40 | >40 | — |

As shown in Table 7, the CBL (the compound 3a) exhibited no significant inhibitory effects on HTEK-293T, GSC-3diff, GSC-12diff, and the 16 tumor cells, indicating an absence of obvious cytotoxic activity against these cells. These results suggest that the CBL has the ability to specifically kill cancer stem cells.

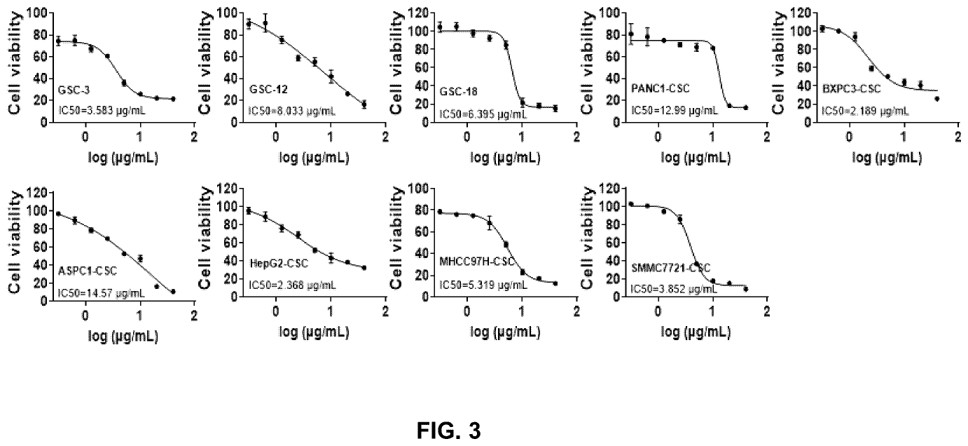

The cell viability of 9 types of cancer stem cells in a CBL solution at different concentrations is shown in FIG. 3. As illustrated in FIG. 3, the cell viability of all 9 types of cancer stem cells decreased with increasing concentrations of the CBL solution, indicating that CBL has a significant effect in killing cancer stem cells.

Based on the above data analysis, the CBL selectively targets and kills cancer stem cells without affecting regular tumor cells, and has the ability to specifically kill stem cells.

### Application Example 3

Preferred cannabicyclol derivatives specifically kill cancer stem cells.

Using the scheme in the application Example 1, cannabicyclol derivatives including compounds 3t, 9a, 9c, 9k, 9l, 9m, 9n, and 13b, were selected as preferred candidates, and the IC₅₀ values of the preferred candidates were summarized in Table 8. As shown in Table 8, the IC₅₀ values of the preferred cannabicyclol derivatives specific against 3 #-GSC glioma stem cells are in a range of 901 to 9.325 μg/mL. In contrast, the IC₅₀ values of the preferred cannabicyclol derivatives against normal cells (293T) are greater than 16.39 μg/mL, and the IC₅₀ values of most of the preferred compounds against normal cells exceed 40 μg/mL. The IC₅₀ values of the preferred cannabicyclol derivatives against tumor cells (U251) are above 13.97 μg/mL, and the IC₅₀ values of some among the preferred compounds against tumor cells exceed 40 μg/mL. These results indicate that a portion of cannabicyclol derivatives possess selective killing of cancer stem cells.

TABLE 8
IC₅₀ values of preferred compounds against cancer
stem cells, tumor cells, and normal cells

| Compound | 3#-GSC(μg/mL) | U251(μg/mL) | 293T(μg/mL) |
|---|---|---|---|
| 3t | 2.995 | 13.97 | 16.39 |
| 9a | 4.315 | >40 | >40 |
| 9c | 9.325 | 24.72 | >40 |
| 9k | 5.370 | >40 | >40 |
| 9l | 2.901 | 24.72 | >40 |
| 9m | 5.436 | >40 | 22.86 |
| 9n | 7.793 | 33.44 | 35.17 |
| 13b | 4.732 | >40 | >40 |

### Application Example 4

The objective of this application example is to explore the effect of the CBL (the compound 3a) on inhibiting the clonal formation of various cancer stem cells (CSCs).

4 g of low-melting-point agarose was dissolved in 12.5 mL of ddH₂O to prepare a 3.2% low-melting-point agarose gel. The low-melting-point agarose gel was sterilized by autoclaving at 121° C. and 0.12 MPa for 20 minutes. After sterilization, the low-melting-point agarose gel was maintained at 60° C. in a drying oven for further use.

1.75 mL of 3.2% sterile low-melting-point agarose gel was taken, 5.25 mL of the corresponding complete medium was added, and the two were mixed thoroughly using a pipette to prepare a base gel at a concentration of 0.8%.

1 mL of the base gel was added to each well of a 6-well plate, ensuring that the base gel evenly covered the bottom of each well. The plate was placed at 4° C. for 5 minutes to allow the gel to solidify. After the gel had fully solidified, the plate was warmed at 37° C. for 5 minutes.

6 cells/cell lines (U251, SMMC7721, GSC-3, GSC-12, SMMC7721-CSC, and HepG2-CSC) were individually digested with 1× trypsin for 3 minutes. The corresponding complete culture medium (same as in the application example 1) was added to terminate the digestion. Cells were centrifuged at 1000 rpm for 3 minutes, and the cells were collected and counted. A total of 5×10³ cells were added to each well, each group was set up with three replicates.

Preparation of an upper layer gel: 900 μL of 3.2% low-melting-point agarose gel and a cell suspension containing 3×10⁴ cells were added to the corresponding complete culture medium (same as in the application example 1), a final volume was 6 mL, resulting in an upper layer gel at a concentration of 0.48%. The upper layer gel was mixed and 1 mL of the upper layer gel was evenly inoculated on each well on top of the base gel (i.e., each well consists of a base gel, an upper layer gel containing cells, and culture medium from bottom to top), cooled and solidified at 4° C. for 5 minutes and rewarmed at 37° C. for 5 minutes.

2 mL of the corresponding complete medium (as described in Application example 1) was added to each well. The plate was then incubated in a cell culture incubator at 37° C. with 5% CO₂ for 6 days. Subsequently, compound 3a solutions at a concentration of 0 μg/mL, 5 μg/mL, and 5 μg/mL, respectively, were added. The culture medium containing the compound 3a solution was refreshed every 3 days. The plate was further cultured in the cell culture incubator for an additional two weeks.

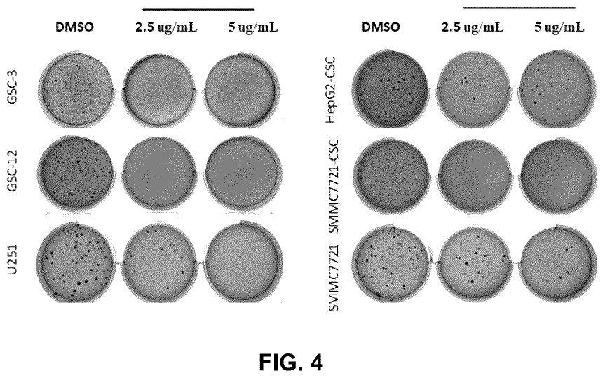

The culture medium was removed after two weeks, and 1 mL of 0.05% crystal violet staining solution (prepared with 4% PFA) was added to each well. The cells were stained for 4 hours, then the staining solution was discarded, and photographs of the cells were taken. The pictures of the cells are shown in FIG. 4. It can be observed that the compound 3a significantly inhibited the colony formation of 4 CSCs (GSC-3, GSC-12, SMMC7721-CSC, HepG2-CSC) and 2 cell lines (glioblastoma cell line U251, liver cancer carcinoma cell line SMMC7721).

### Application Example 5

The purpose of this application example is to explore the inhibitory effect of CBL on liver cancer.

Establishment of a mouse liver cancer model: 2×10⁶ SMMC7721 (containing 30% Matrigel) was subcutaneously injected into the right inguinal region of the mice (NOD/ShiLtJGpt-Prkdc^{em26cd52} Il2rg^{em26cd22}/Gpt(NCG)mic). The model was established when the tumor volume reached 50 mm³.

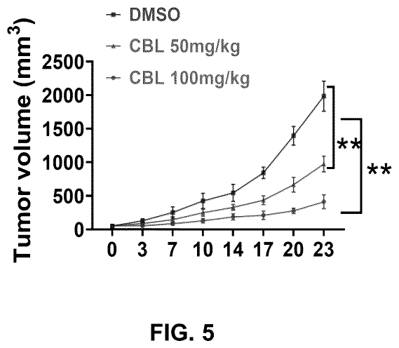

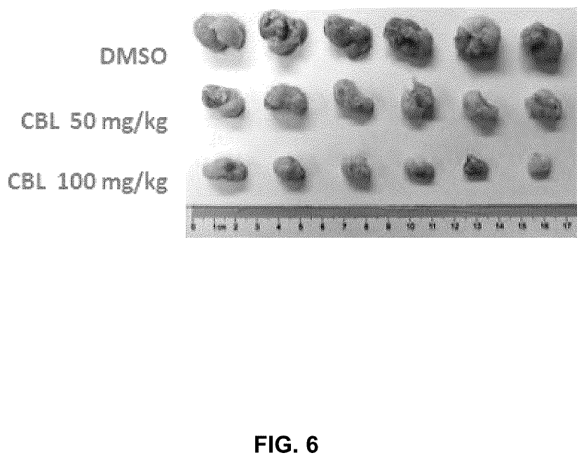

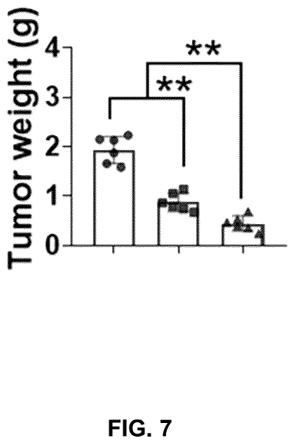

According to the weight of mice: in treatment group 1, each mouse was intraperitoneally injected with a CBL solution at 50 mg/kg, and in treatment group 2, each mouse was intraperitoneally injected with a CBL solution at 100 mg/kg. The mice in a control group were injected with an equal volume of a DMSO solvent. The drug was administered once every 2 days, continuing for 23 days. Tumor volume was measured on days 3, 7, 10, 14, 17, 20, and 23. On day 23, the tumor tissue was collected and weighed. The tumor volume changes at different time points are shown in FIG. 5, the tumor tissue collected on day 23 is shown in FIG. 6, and the average tumor weight of different treatment groups is shown in FIG. 7.

The results show that, compared with the control group, the tumor volume and weight in treatment group 1 and treatment group 2 were significantly inhibited, indicating that CBL can effectively suppress liver cancer growth in vivo, demonstrating a significant anti-liver cancer effect. Moreover, the 100 mg/kg CBL dosage had a better inhibitory effect on liver cancer compared to the 50 mg/kg dosage, and the statistical results showed a significant difference.

As shown in the application examples, cannabicyclol and cannabicyclol derivatives exhibit significant and specific killing effects on various cancer stem cells in vitro, and can also inhibit the clonal formation of various cancer stem cells. Therefore, cannabicyclol and cannabicyclol derivatives can be used to develop a medicine that targets and kills cancer stem cells or inhibits the clonal formation of cancer stem cells, with the potential to become a specific anti-tumor medicine targeting cancer stem cells. Additionally, through in vivo mouse studies, it was found that cannabicyclol and cannabicyclol derivatives can effectively suppress liver cancer growth in mice, demonstrating significant anti-liver cancer effects. Therefore, cannabicyclol and cannabicyclol derivatives have the potential to treat various cancers by targeting and killing cancer stem cells.

The foregoing is only a preferred embodiment of the present disclosure, and it should be pointed out that, for a person of ordinary skill in the art, a number of improvements and embellishments may be made without departing from the principles of the present disclosure, which shall also be considered as the scope of protection of the present disclosure.

What is claimed is:

1. A cannabicyclol derivative, comprising a structural formula as follows: 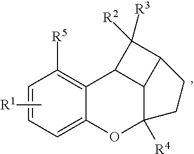
wherein
R¹ is selected from hydrogen, substituted or unsubstituted linear alkyl, substituted or unsubstituted branched alkyl, substituted or unsubstituted aryl, and substituted or unsubstituted heteroaryl,
R² and R³ are independently selected hydrogen, substituted or unsubstituted linear alkyl, substituted or unsubstituted branched alkyl, substituted or unsubstituted alkenyl, substituted or unsubstituted ester, substituted or unsubstituted heteroalkyl, substituted or unsubstituted aryl, and substituted or unsubstituted heteroaryl,
R⁴ is selected from hydrogen, substituted or unsubstituted linear alkyl, substituted or unsubstituted branched alkyl, substituted or unsubstituted aryl, or substituted or unsubstituted heteroaryl, and
R⁵ is selected from hydrogen, hydroxy, substituted or unsubstituted linear alkyl, substituted or unsubstituted branched alkyl, substituted or unsubstituted aryl, or substituted or unsubstituted heteroaryl.

2. The cannabicyclol derivative of claim 1, wherein when there is one or more substituents in R¹, R², R³, R⁴, and R⁵,
the one or more substituents are independently selected from linear alkyl or branched alkyl containing 1 to 13 carbon atoms, halogen atom, linear alkoxy or branched alkoxy containing 1 to 13 carbon atoms, cyano, amino, amido, hydroxyl, ester, alkenyl, alkynyl, cycloalkyl containing 3 to 10 carbon atoms, heterocycloalkyl containing 3 to 10 carbon atoms, substituted or unsubstituted aryl, and substituted or unsubstituted heteroaryl.

3. The cannabicyclol derivative of claim 2, wherein R¹, R², R³, R⁴, and R⁵ are independently selected from following structures: 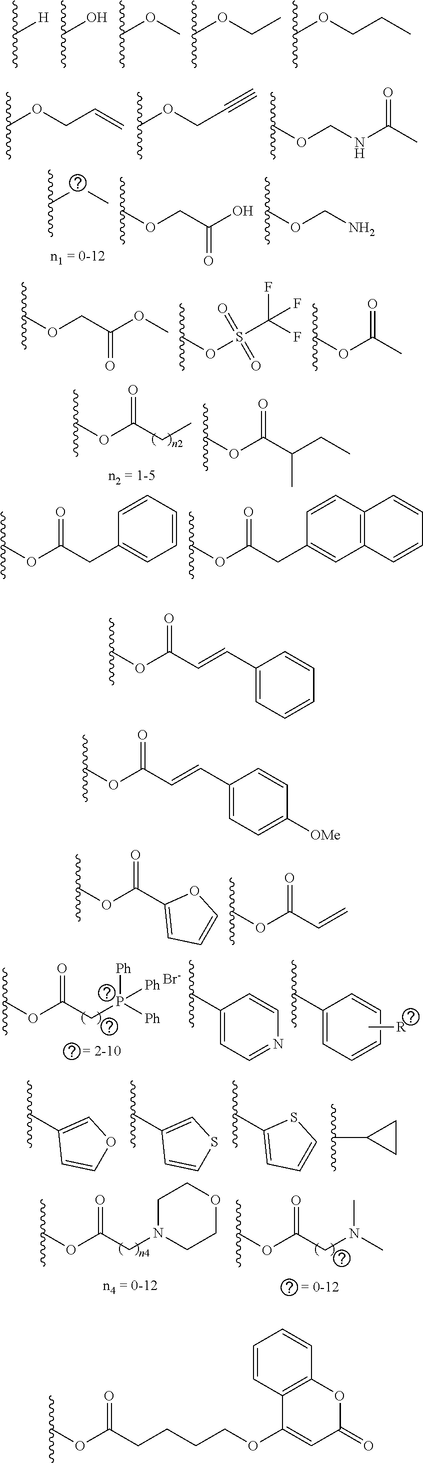 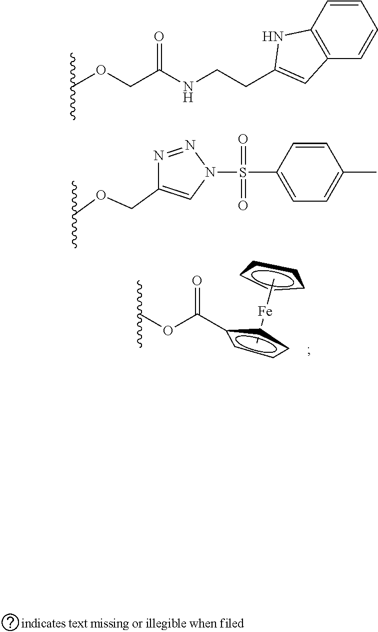
wherein R⁶ is selected from following structures H, Me, Et, n-Pr, i-Pr, n-Bu, i-Bu, t-Bu, OMe, F, Cl, Br, and allyl.

4. The cannabicyclol derivative of claim 1, selecting from compounds of following structural formulas:

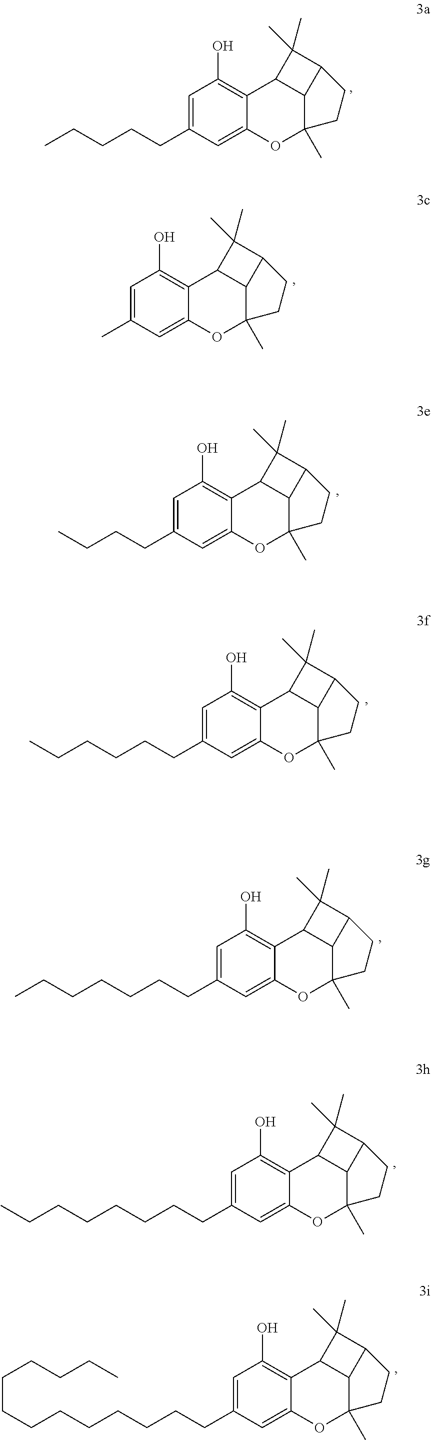

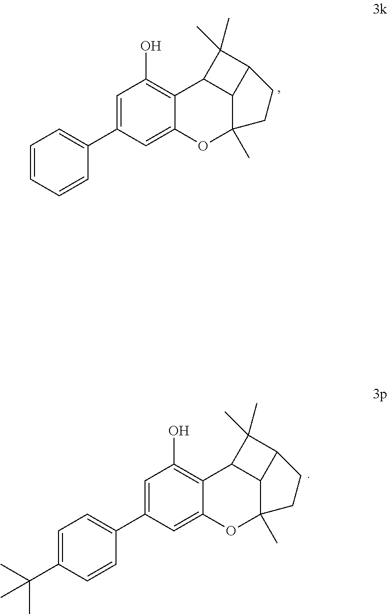 [SMILES: CC12CCC3C1C(c1c(O)cc(-c4ccccc4)cc1O2)C3(C)C ; CC(C)(C)c1ccc(-c2cc(O)c3c(c2)OC2(C)CCC4C2C3C4(C)C)cc1]

5. The cannabicyclol derivative of claim 1, selecting from compounds of following structural formulas, the following structural formulas being 3b, 3d, 3j, 3l to 3o, and 3q to 3z in sequence:

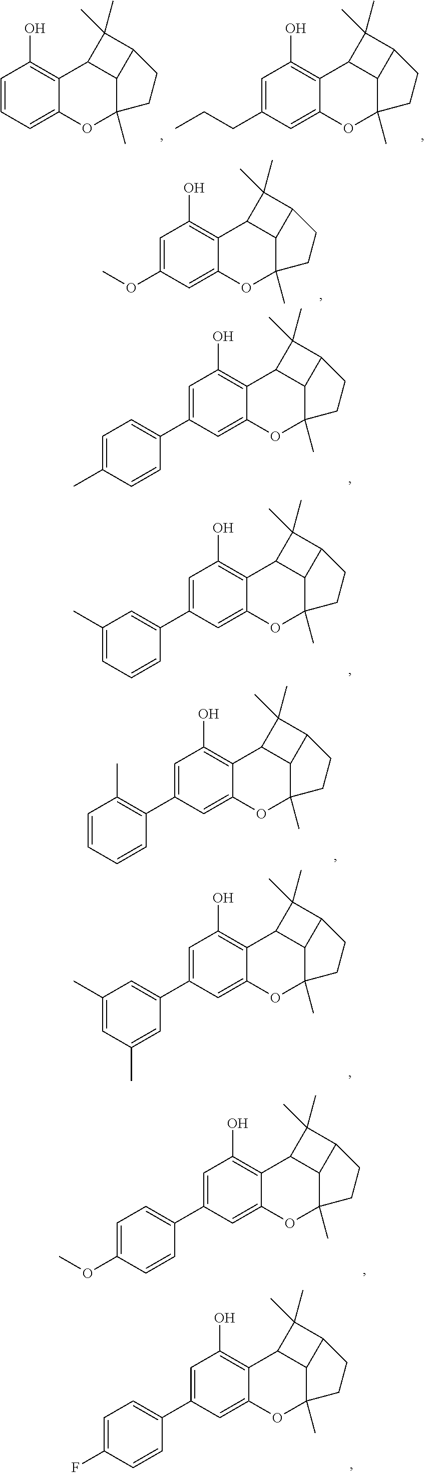

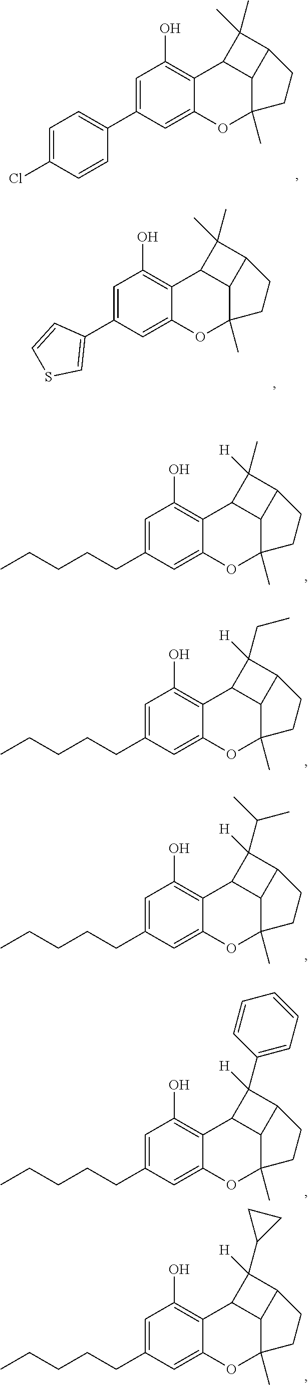

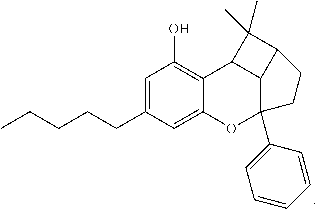 [SMILES: CCCCCc1cc(O)c2c(c1)OC1(c3ccccc3)CCC3C1C2C3(C)C]

6. The cannabicyclol derivative of claim 1, comprising a compound of a following structural formula 5:

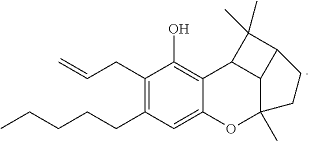 [SMILES: C=CCc1c(CCCCC)cc2c(c1O)C1C3C(CCC3(C)O2)C1(C)C]

7. The cannabicyclol derivative of claim 1, selecting from compounds of following structural formulas, the following structural formulas being 7a to 7i in sequence:

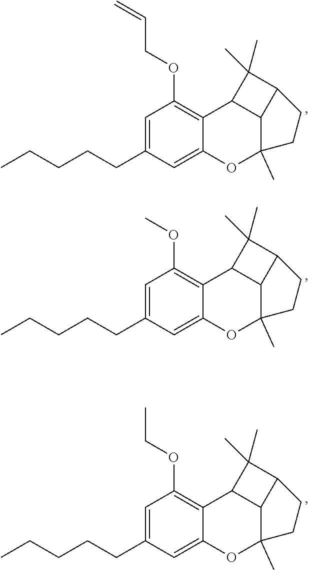

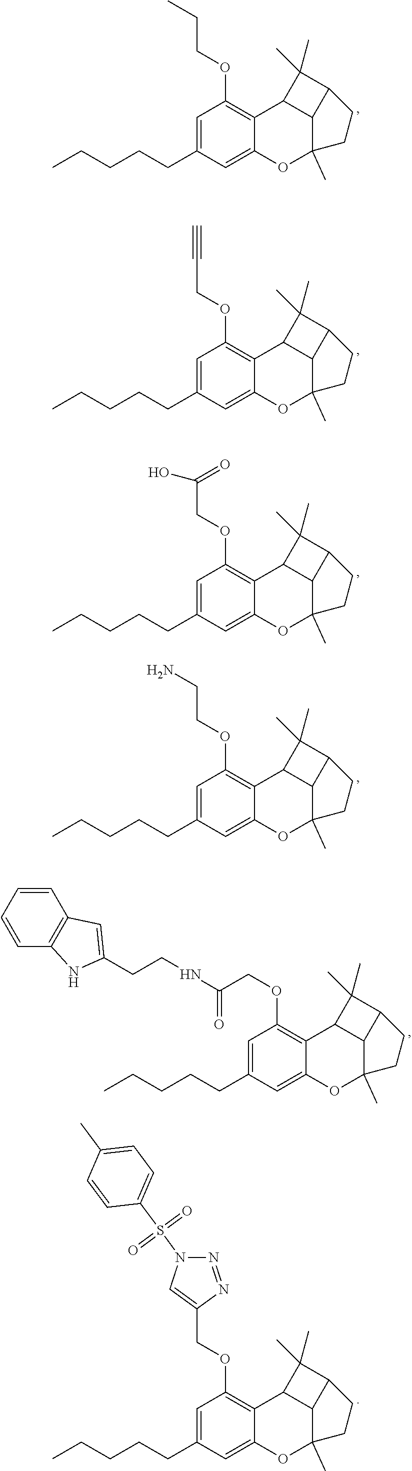

8. The cannabicyclol derivative of claim 1, selecting from compounds of following structural formulas, the following structural formulas being 9a to 9t in sequence:

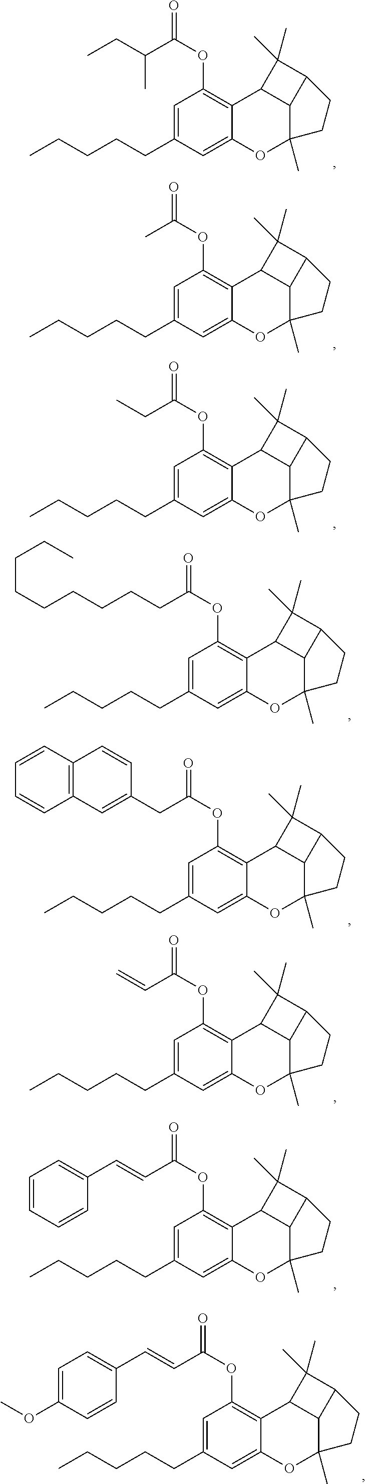

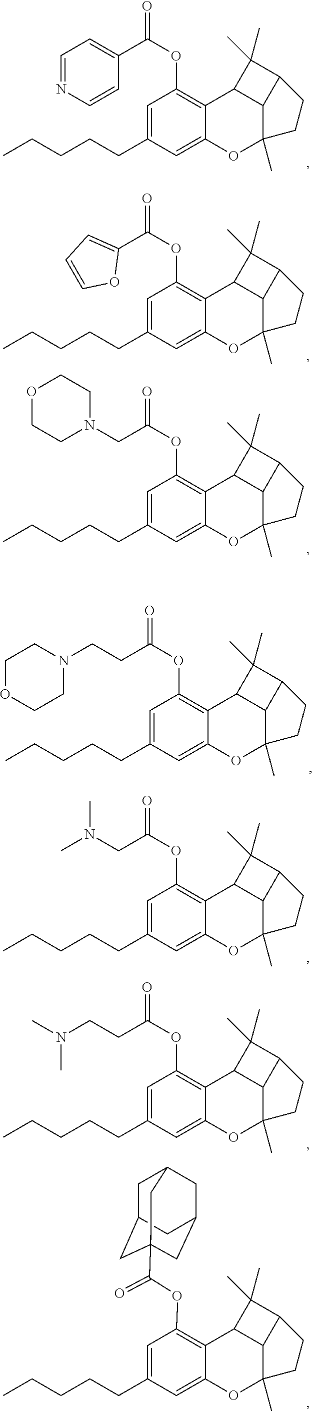

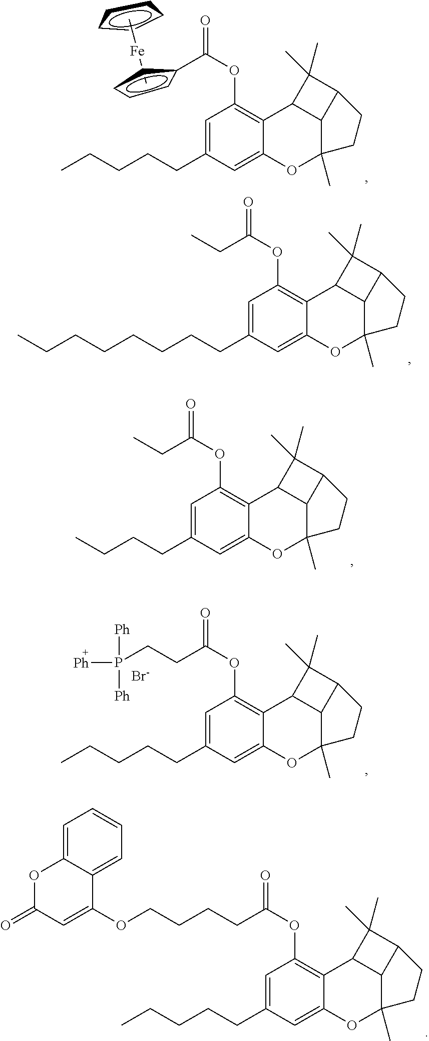

9. The cannabicyclol derivative of claim 1, selecting from compounds of following structural formulas, the following structural formulas being 13a to 13d in sequence:

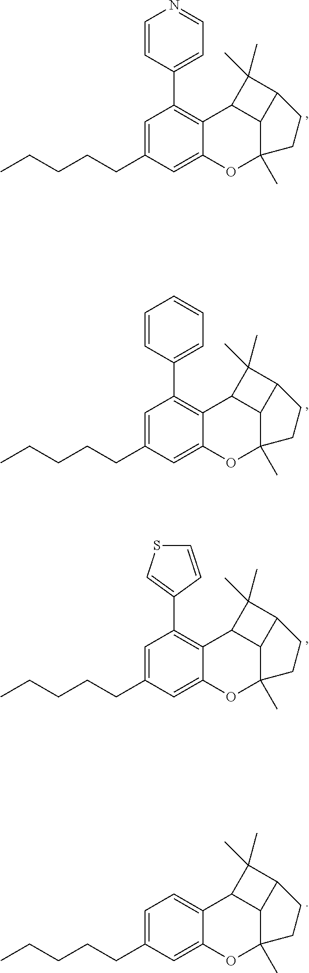 [SMILES: CCCCCc1cc2c(c(-c3ccncc3)c1)C1C3C(CCC3(C)O2)C1(C)C ; CCCCCc1cc2c(c(-c3ccccc3)c1)C1C3C(CCC3(C)O2)C1(C)C ; CCCCCc1cc2c(c(-c3ccsc3)c1)C1C3C(CCC3(C)O2)C1(C)C ; CCCCCc1ccc2c(c1)OC1(C)CCC3C1C2C3(C)C]

10. The cannabicyclol derivative of claim 4, comprising a formula III: 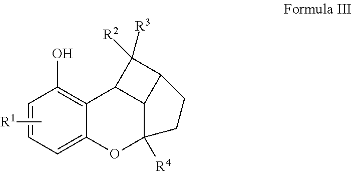
wherein, the cannabicyclol derivative of the formula III is prepared by:
dissolving a compound of a formula I, a compound of a formula II, and ethylenediamine in toluene for reaction, and then conducting a photocatalytic reaction under a protective atmosphere and an action of a photocatalyst to obtain the cannabicyclol derivative of the formula III, 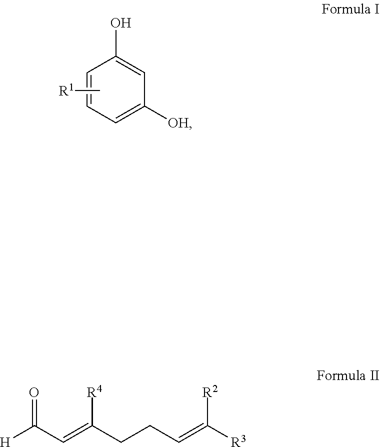
wherein
R¹ is selected from pentyl, R² is selected from methyl, R³ is selected from methyl, and R⁴ is selected from methyl;
R¹ is selected from methyl, R² is selected from methyl, R³ is selected from methyl, and R⁴ is selected from methyl;
R¹ is selected from butyl, R² is selected from methyl, R³ is selected from methyl, and R⁴ is selected from methyl;
R¹ is selected from hexyl, R² is selected from methyl, R³ is selected from methyl, and R⁴ is selected from methyl;
R¹ is selected from heptyl, R² is selected from methyl, R³ is selected from methyl, and R⁴ is selected from methyl;
R¹ is selected from octyl, R² is selected from methyl, R³ is selected from methyl, and R⁴ is selected from methyl;
R¹ is selected from n-tridecyl, R² is selected from methyl, R³ is selected from methyl, and R⁴ is selected from methyl;
R¹ is selected from phenyl, R² is selected from methyl, R³ is selected from methyl, and R⁴ is selected from methyl; or
R¹ is selected from 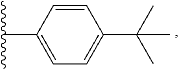 [SMILES: *c1ccc(C(C)(C)C)cc1]
R² is selected from methyl, R³ is selected from methyl, and R⁴ is selected from methyl.

11. The cannabicyclol derivative of claim 5, comprising a formula III: 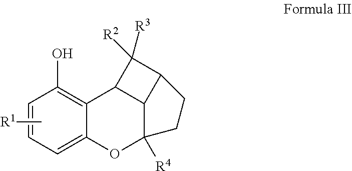
wherein, the cannabicyclol derivative of the formula III is prepared by:
dissolving a compound of a formula I, a compound of a formula II, and ethylenediamine in toluene for reaction, and then conducting a photocatalytic reaction under a protective atmosphere and an action of a photocatalyst to obtain the cannabicyclol derivative of the formula III, 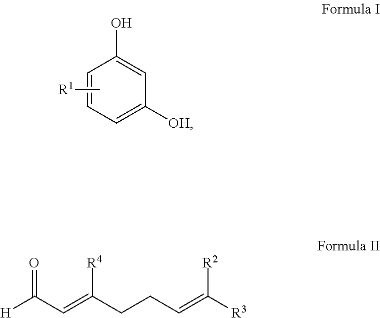
wherein
R¹ is selected from H, R² is selected from methyl, R³ is selected from methyl, and R⁴ is selected from methyl;
R¹ is selected from propyl, R² is selected from methyl, R³ is selected from methyl, and R⁴ is selected from methyl;
R¹ is selected from methoxy, R² is selected from methyl, R³ is selected from methyl, and R⁴ is selected from methyl;
R¹ is selected from p-tolyl, R² is selected from methyl, R³ is selected from methyl, and R⁴ is selected from methyl;
R¹ is selected from m-tolyl, R² is selected from methyl, R³ is selected from methyl, and R⁴ is selected from methyl;
R¹ is selected from o-tolyl, R² is selected from methyl, R³ is selected from methyl, and R⁴ is selected from methyl;
R¹ is selected from 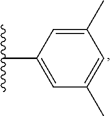 [SMILES: *c1cc(C)cc(C)c1]
R² is selected from methyl, R³ is selected from methyl, and R⁴ is selected from methyl;
R¹ is selected from 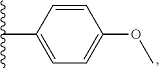 [SMILES: *c1ccc(OC)cc1]
R² is selected from methyl, R³ is selected from methyl, and R⁴ is selected from methyl;
R¹ is selected from 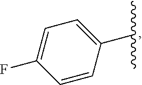 [SMILES: *c1ccc(F)cc1]
R² is selected from methyl, R³ is selected from methyl, and R⁴ is selected from methyl;
R¹ is selected from 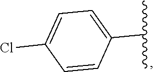 [SMILES: *c1ccc(Cl)cc1]
R² is selected from methyl, R³ is selected from methyl, and R⁴ is selected from methyl;
R¹ is selected from 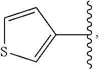 [SMILES: *c1ccsc1]
R² is selected from methyl, R³ is selected from methyl, and R⁴ is selected from methyl;
R¹ is selected from pentyl, R² is selected from H, R³ is selected from methyl, and R⁴ is selected from methyl;
R¹ is selected from pentyl, R² is selected from H, R³ is selected from ethyl, R⁴ is selected from methyl;
R¹ is selected from pentyl, R² is selected from H, R³ is selected from isopropyl, and R⁴ is selected from methyl;
R¹ is selected from pentyl, R² is selected from H, R³ is selected from phenyl, and R⁴ is selected from methyl;
R¹ is selected from pentyl, R² is selected from H, R³ is selected from cyclopropyl, and R⁴ is selected from methyl; or
R¹ is selected from pentyl, R² is selected from methyl, R³ is selected from methyl, and R⁴ is selected from phenyl.

12. The cannabicyclol derivative of claim 6, wherein the compound 5 is prepared by:
reacting cannabinophene derivative and 2,3-dichloro-5,6-dicyanobenzoquinone under an action of indium trifluoromethanesulfonate to obtain a colorless oily liquid, the colorless oily liquid being the compound 5, wherein a structural formula of the cannabinophene derivative is:

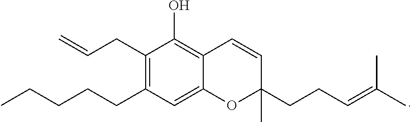 [SMILES: C=CCc1c(CCCCC)cc2c(c1O)C=CC(C)(CCC=C(C)C)O2]

13. The cannabicyclol derivative of claim 7, wherein
the compounds 7a to 7g are prepared by:
mixing a compound 3a of a structural formula 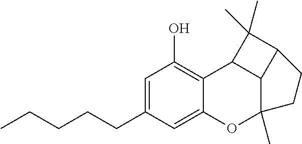 [SMILES: CCCCCc1cc(O)c2c(c1)OC1(C)CCC3C1C2C3(C)C]
with a halogen-containing compound and reacting under an action of a base, wherein the halogen-containing compound includes allyl bromide, halomethane, halogenated ethane, 1-halopropane, 3-bromopropyne, ethyl bromoacetate, and 2-(Boc-amino)ethyl bromide;
the compound 7h is prepared by:
mixing the compound 7f, tryptamine, 1-(3-dimethylaminopropyl)-3-ethylcarbodiimide hydrochloride, 1-hydroxybenzotriazole, and triethylamine and reacting to obtain a colorless oily liquid, the colorless oily liquid being the compound 7h; and
the compound 7i is prepared by:
mixing the compound 7e, p-toluenesulfonyl azide, and thiophene-2-carboxylate Cuprous and reacting to obtain a colorless oily liquid, and colorless oily liquid being the compound 7i.

14. The cannabicyclol derivative of claim 8, wherein
the compounds 9a to 9p are prepared by:
reacting a compound 3a of a structural formula 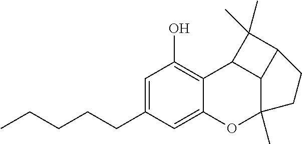 [SMILES: CCCCCc1cc(O)c2c(c1)OC1(C)CCC3C1C2C3(C)C]
and an acid under an action of a catalyst to obtain a colorless oily liquid, the colorless oily liquid being the compounds 9a to 9p, wherein a structural formula of the acid is 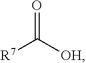
and R⁷ selects from 2-Methylbutyl, n-decyl, 2-Naphthylethyl, 3-Phenylpropenyl, (E)-4-Methoxy-3-phenylpropenyl, 4-Pyridylmethyl, 2-Furylmethyl, 4-Morpholinoethyl, 3-(4-Morpholinyl)propyl, N,N-Dimethylethyl, 3-(Dimethylamino)propyl, 1-Adamantylmethyl, and Ferrocene methyl, and the acid further includes acetic anhydride, propionyl chloride, acryloyl chloride, or propionic anhydride;
the compound 9q is prepared by:
reacting a compound 3h of a structural formula 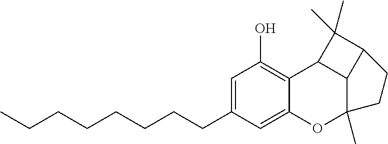 [SMILES: CCCCCCCCc1cc(O)c2c(c1)OC1(C)CCC3C1C2C3(C)C]
and propionic anhydride under an action of a catalyst to obtain the compound 9q;
the compound 9r is prepared by:
reacting a compound 3e of a structural formula 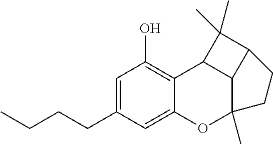 [SMILES: CCCCc1cc(O)c2c(c1)OC1(C)CCC3C1C2C3(C)C]
and propionic anhydride under an action of a catalyst to obtain the compound 9r; and
a compound 9s and a compound 9t are prepared by:
reacting the compound 3a and 5-Bromopentanoic acid under an action of a catalyst to obtain an intermediate product, and reacting the intermediate product with a substituted compound to obtain the compound 9s and the compound 9t, the substituted compound including triphenylphosphine or 4-Hydroxycoumarin.

15. The cannabicyclol derivative of claim 9, wherein
the compounds 13a to 13c are prepared by:
mixing a compound 3a of a structural formula 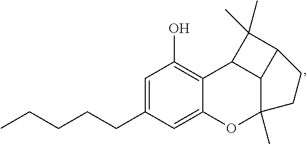 [SMILES: CCCCCc1cc(O)c2c(c1)OC1(C)CCC3C1C2C3(C)C]
triflic anhydride, and pyridine in a solvent for reaction to obtain an intermediate product, and reacting the intermediate product with a substituted boric acid under an action of a catalyst, wherein, a structural formula of the intermediate product is: 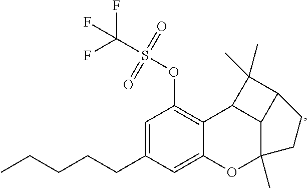 [SMILES: CCCCCc1cc2c(c(OS(=O)(=O)C(F)(F)F)c1)C1C3C(CCC3(C)O2)C1(C)C]
a structural formula of the substituted boric acid is: 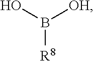
and R8 is pyridine, phenyl, or thiophene; and
the compound 13d is prepared by:
mixing the compound 3a, the triflic anhydride, and the pyridine in a solvent for reaction to obtain the intermediate product, and mixing the intermediate product, 1,1′-bis(diphenylphosphino) ferrocene, palladium acetate, formic acid, and triethylamine in a solvent for reaction to obtain the compound 13d.

16. The cannabicyclol derivative of claim 10, wherein
the photocatalyst includes one or more of [Ir{dFCF₃ppy}₂(bpy)]PF₆, [Ir{dFCF₃ppy}₂(dtbbpy)]PF₆, [Ir(ppy)₂(dtbbpy)]PF₆, fac-Ir(ppy)₃, [Ru(bpy)₃]Cl₂, [Ru(bpy)₃](PF₆)₂, and Eosin Y,
a light condition for a photocatalytic reaction includes a wavelength ranging from 365 nm to 560 nm, and an illumination time ranging from 30 min to 5 h, and
a molar ratio of the compound of the formula I, the compound of the formula II, the ethylenediamine, and the photocatalyst is 1:1:(0.01 to 0.1):(0.005 to 0.02).

17. The cannabicyclol derivative of claim 11, wherein
the photocatalyst includes one or more of [Ir{dFCF₃ppy}₂(bpy)]PF₆, [Ir{dFCF₃ppy}₂(dtbbpy)]PF₆, [Ir(ppy)₂(dtbbpy)]PF₆, fac-Ir(ppy)₃, [Ru(bpy)₃]Cl₂, [Ru(bpy)₃](PF₆)₂, and Eosin Y,
a light condition for a photocatalytic reaction includes a wavelength ranging from 365 nm to 560 nm, and an illumination time ranging from 30 min to 5 h, and
a molar ratio of the compound of the formula I, the compound of the formula II, the ethylenediamine, and the photocatalyst is 1:1:(0.01 to 0.1):(0.005 to 0.02).

18. The cannabicyclol derivative of claim 13, wherein
a molar ratio of the compound 3a, the halogen-containing compound, and the base is 1:2:(1-5),
a reaction temperature is in a range of 60° C. to 70° C., and
a reaction time is in a range of 5 h to 10 h.

19. The cannabicyclol derivative of claim 14, wherein
a molar ratio of the compound 3a, the acid, and the catalyst is 1:(1-2):(1-3),
a reaction temperature is in a range of 20° C. to 40° C.; and
a reaction time is in a range of 5 h to 10 h.

20. A medicine comprising the cannabicyclol derivative of claim 1 and a pharmaceutically acceptable excipient, wherein the pharmaceutically acceptable excipient includes at least one of a diluent, an excipient, a filler, a binder, a humectant, a disintegrant, an absorption promoter, a surfactant, an adsorptive support, or a lubricant.
